(19) United States
(12) Patent Application Publication
(10) Pub. No.: US 2022/0249517 A1
(43) Pub. Date: Aug. 11, 2022

(54) COMPOSITIONS AND METHODS FOR TREATING NONINFECTIOUS UVEITIS

(71) Applicant: CLEARSIDE BIOMEDICAL, INC., Alpharetta, GA (US)

(72) Inventors: Daniel WHITE, Alpharetta, GA (US); Glenn NORONHA, Alpharetta, GA (US); Jennifer KISSNER, Alpharetta, GA (US)

(21) Appl. No.: 17/478,112

(22) Filed: Sep. 17, 2021

Related U.S. Application Data
(63) Continuation of application No. 16/292,845, filed on Mar. 5, 2019, now abandoned.
(60) Provisional application No. 62/793,562, filed on Jan. 17, 2019.
(60) Provisional application No. 62/638,744, filed on Mar. 5, 2018.

(51) Int. Cl. A61K 31/58; A61K 9/00; A61P 27/02; A61M 37/00; A61K 47/02; A61K 47/26; A61K 47/12; A61K 45/06; A61K 47/38

(52) U.S. Cl. CPC A61K 31/58; A61K 9/0019; A61P 27/02; A61M 37/0015; A61K 9/0051; A61M 2037/0023; A61K 47/26; A61K 47/12; A61K 9/0048; A61K 45/06; A61K 47/38; A61K 47/02

(57) ABSTRACT

The present invention relates to methods, devices, and compositions for treating ocular disorders such as uveitis, macular edema associated with uveitis, and diabetic macular edema. For example, the methods include treatment of subjects having macular edema associated with non-infectious uveitis, or diabetic macular edema, by administering to the subjects a triamcinolone composition via non-surgical administration to the suprachoroidal space (SCS) of the eye.

### CROSS-REFERENCE TO RELATED APPLICATIONS

This application is a continuation of U.S. Utility application Ser. No. 16/292,845, filed Mar. 5, 2019 which claims priority to U.S. Provisional Application No. 62/638,744, filed Mar. 5, 2018, and U.S. Provisional Application No. 62/793,562, filed Jan. 17, 2019, the contents of each of which are hereby incorporated by reference in their entireties.

### BACKGROUND OF THE INVENTION

Prolonged or severe inflammation in the back of the eye can result in the breakdown of cells at the interface of the retina and choroid, resulting in the leakage and accumulation of fluid in the macular region of the retina. This build-up of fluid can cause abnormal swelling of the macula, or macular edema, which can rapidly result in distortion of vision and eventually blindness. Because of the macula's critical role in central vision, macular edema can rapidly result in distortion of vision and eventually blindness.

Macular edema is the most frequent cause of visual impairment among patients with uveitis. Uveitis is the most common form of inflammation of the choroid and surrounding tissues in the eye, and one of the most frequent causes of blindness in the developed world. Uveitis, which can affect both eyes and is often initially diagnosed in individuals 20 to 50 years, currently accounts for 10% of vision loss/blindness in the United States and 15% worldwide, mainly occurring in the 20-50 year age group. According to studies measuring incidence and prevalence of uveitis, more than 160,000 people are diagnosed with uveitis in the United States each year. Uveitis can be infectious, meaning it is caused by an immune response to fight an infection inside the eye, or non-infectious. Non-infectious uveitis accounts for approximately 80% of all uveitis cases. Because uveitis can become chronic or recurrent if not adequately treated, some patients may become refractory, or unresponsive, to treatment, leading to irreversible blindness. Further, macular edema may persist even with successful control of the inflammatory response.

Corticosteroids are currently regarded as the most effective treatment for non-infectious uveitis. Although used to treat some uveitis patients, corticosteroid eye drops are generally ineffective in treating patients with macular edema associated with uveitis because they cannot reach the choroid and retina in effective concentrations. Oral or other systemically administered corticosteroids and immunosuppressive agents can be effective in treating patients with macular edema associated with uveitis, but their long-term use is associated with harmful side effects.

Diabetic macular edema (DME) is caused by diabetic retinopathy, which is the most common diabetic eye disease and the leading cause of vision loss in the United States. Anti-VEGF agents are often used to treat DME. However, anti-VEGF treatments are associated with complications relating to repeated administration, and insufficient or only modest improvements in visual acuity.

There is a need in the art for improved methods for treating uveitis, macular edema associated with uveitis, diabetic macular edema, and other posterior ocular disorders. This disclosure addresses these and other needs.

### SUMMARY OF THE INVENTION

This invention is generally related to ophthalmic therapies, and more particularly to methods, devices, and compositions that allow for infusion of a fluid drug formulation into posterior ocular tissues for targeted, localized treatment of posterior ocular disorders. For example, the compositions, devices, and methods provided herein allow for the treatment of uveitis and/or macular degeneration associated with uveitis.

In some embodiments, the present disclosure provides methods for treating macular edema associated with uveitis in a subject in need thereof, the method comprising non-surgically administering an effective amount of a triamcinolone drug formulation to the suprachoroidal space (SCS) of the eye of the human subject in need of treatment. In some embodiments, the effective amount of triamcinolone in the drug formulation is about 4 mg. In some embodiments, the method comprises administering two doses of the triamcinolone drug formulation to the SCS of the eye of the subject. In some embodiments, the method comprises administering two or more doses of the triamcinolone drug formulation to the SCS of the eye of the subject. In some embodiments, the two or more doses of the triamcinolone drug formulation are administered about 1, 2, 3, 4, 5, 6, 7, 8, 9, 10, 11, 12, 13, 14, 15, 16, or 17 weeks apart. In some embodiments, the two doses of the triamcinolone drug formulation are administered about 12 weeks apart. In some embodiments, the two doses of the triamcinolone drug formulation are administered about 12 weeks apart, and additional doses of the triamcinolone drug formulation are subsequently administered.

In some embodiments, the uveitis is non-infectious uveitis. In some embodiments, the non-infectious uveitis is selected from the group consisting of pan, anterior, intermediate, or posterior uveitis. In some embodiments, the subject has a visual acuity score of between about 5 and about 70 letters read before administration of the triamcinolone drug formulation.

In some embodiments, the method decreases retina thickness and/or macula thickness relative to a baseline measurement prior to treatment of the subject with the triamcinolone drug formulation. In some embodiments, the method of claim 1, wherein the method decreases retina thickness and/or macula thickness relative to a subject that did not receive the triamcinolone drug formulation. In further embodiments, the retinal thickness is decreased by at least about 20 μm, at least about 40 μm, at least about 50 μm, at least about 100 μm, at least about 125 μm, least about 150 μm, at least about 175 μm or at least about 200 μm. In some embodiments, the method results in an improvement in CST of at least 10%, at least 20%, at least 25%, at least 40%, at least 50%, at least 75%, at least 90%, or more. In some embodiments, the method resolves the increased retinal thickness in the subject. Thus, in some embodiments, the method resolves macular edema in the subject. In some embodiments, the method results in resolution of macular edema by about week 2, about week 3, about week 4, about week 5, about week 6, about week 7, or about week 8 following administration of the first dose of triamcinolone drug formulation to the SCS. In some embodiments, the resolution of macular edema in the subject is maintained for at least about 12 weeks, at least about 18 weeks, at least about 24 weeks, at least about 30 weeks, at least about 36 weeks, at least about 42 weeks, at least about 48 weeks, or longer following administration of the first dose of triamcinolone drug formulation to the SCS.

In some embodiments, the method increases a visual acuity score of the subject relative to a baseline measurement prior to treatment of the subject with triamcinolone drug formulation. In some embodiments, the visual acuity score is Best Corrected Visual Acuity (BCVA). In some embodiments, the method increases the BCVA of the subject relative to a subject that did not receive the triamcinolone drug formulation. In some embodiments, the BCVA is assessed using an Early Treatment of Diabetic Retinopathy Study (ETDRS) visual acuity charts protocol. In some embodiments, the increase in the BCVA is a gain of about 5, about 6, about 7, about 8, about 9, about 10, about 12, about 14, about 15, about 16, about 17, about 18, about 19, about 20, or more letters. In some embodiments, the increase in the BCVA is a gain of at least 15 letters. In some embodiments, the increase in the BCVA is maintained for at least about 24 weeks, at least about 30 weeks, at least about 36 weeks, at least about 40 weeks, at least about 48 weeks, or longer following the first dose of triamcinolone drug formulation to the SCS. In some embodiments, the increase in the BCVA is maintained for at least about 24 weeks, at least about 30 weeks, at least about 36 weeks, at least about 40 weeks, at least about 48 weeks, or longer following the last dose of triamcinolone drug formulation to the SCS.

In some embodiments, the method reduces inflammation in the eye of the subject. In some embodiments, the method reduces vitreous haze, anterior chamber flare, and/or inflammatory cells in the anterior chamber. In some embodiments, the method reduces the inflammatory score in the eye of the subject by at least about 50%, at least about 75%, at least about 90%, at least about 95%, or by 100%. In some embodiments, the method resolves inflammation in the eye of the subject.

In some embodiments, the triamcinolone drug formulation is administered to the subject by suprachoroidal injection comprising the use of an SCS microinjector. In further embodiments, the SCS microinjector comprises a 30 gauge needle that is about 900 μm or about 1100 μm in length.

In one aspect, the present disclosure provides methods for achieving a durable clinical outcome in a subject having macular edema, the method comprising non-surgically administering an effective amount of a first dose of a triamcinolone drug formulation to the suprachoroidal space (SCS) of the eye of the subject. In some embodiments, the subject has macular edema associated with uveitis. In some embodiments, the durable outcome comprises a reduction in retinal thickness in the eye of the subject. In further embodiments, the reduction in retinal thickness in the eye of the subject is a reduction in retinal thickness of at least about 20 μm, at least about 40 μm, at least about 50 μm, at least about 100 μm, at least about 150 μm, or at least about 200 μm. In some embodiments, the durable clinical outcome comprises an increase in the visual acuity score of the subject relative to a baseline measurement prior to treatment of the subject with triamcinolone drug formulation. In further embodiments, the increase in the visual acuity score is a gain of about 5, about 6, about 7, about 8, about 9, about 10, about 12, about 14, about 15, or more letters. In some embodiments, the visual acuity is measured by Best Corrected Visual Acuity (BCVA). In some embodiments, the durable clinical outcome comprises a reduction of inflammation in the eye of the subject. In some embodiments, the reduction of inflammation comprises a reduction in an inflammatory score of at least about 50%, at least about 75%, or at least about 90%, or at least about 95%. In some embodiments, the reduction of inflammation comprises a reduction in vitreous haze, anterior chamber flare, and/or inflammatory cells in the anterior chamber.

In some embodiments, the methods provided result in durable clinical outcome comprising a reduction in retinal thickness in the eye of the subject, an increase in the visual acuity score of the subject, and a reduction of inflammation in the eye of the subject. In some embodiments, the method results in resolution of macular edema. In some embodiments, the method results in resolution of inflammation in the eye. In some embodiments, the durable clinical outcome is maintained for at least 24 weeks, at least 30 weeks, at least 36 weeks, at least 42 weeks, at least 48 weeks, at least 54 weeks, or longer.

In some embodiments, the present disclosure provides a method of diabetic macular edema (DME) in a subject in need thereof, the method comprising non-surgically administering an effective amount of a triamcinolone drug formulation to the suprachoroidal space (SCS) of the eye of the subject and administering an effective amount of a VEGF modulator to the eye of the subject. In some embodiments, the effective amount of triamcinolone in the drug formulation is about 4 mg. In some embodiments, the VEGF modulator is a VEGF antagonist. In further embodiments, the VEGF antagonist is aflibercept. In some embodiments, the VEGF modulator is administered to the eye via non-surgical administration to the SCS. In further embodiments, the VEGF modulator is administered to the eye via intravitreal injection. In some embodiments, the triamcinolone drug formulation and/or the VEGF modulator is administered to the SCS via an SCS microinjector.

In some embodiments, the subject has a visual acuity score of between about 5 and about 70 letters read before administration of the triamcinolone drug formulation and/or the VEGF modulator. In some embodiments, the method decreases retina thickness and/or macula thickness relative to a baseline measurement prior to treatment of the subject with the triamcinolone drug formulation and/or VEGF modulator, and/or relative to a subject who received the VEGF modulator alone. In some embodiments, the method decreases retina thickness and/or macula thickness relative to a subject that did not receive the triamcinolone drug formulation and/or VEGF modulator, and/or relative to a subject who received the VEGF modulator alone. In further embodiments, the retinal thickness is decreased by at least about 20 μm, at least about 40 μm, at least about 50 μm, at least about 100 μm, at least about 125 μm, at least about 150 μm, at least about 175 μm, or at least about 200 μm. In some embodiments, the method results in an improvement in CST of at least 10%, at least 20%, at least 25%, at least 40%, at least 50%, at least 75%, at least 90%, or more. In some embodiments, the method results in resolution of the elevated central retinal thickness in the subject. In some embodiments, the method increases a Best Corrected Visual Acuity (BCVA) of the subject relative to a baseline measurement prior to treatment of the subject with triamcinolone drug formulation and/or VEGF modulator, and/or or relative to a subject who received the VEGF modulator alone. In further embodiments, the BCVA is assessed using an ETDRS visual acuity charts protocol. In some embodiments, the increase in the BCVA is a gain of about 5, about 6, about 7, about 8, about 9, about 10, about 12, about 14, about 15, about 16, about 17, about 18, about 19, about 20, or more letters. In further embodiments, the increase in the BCVA is a gain of at least 10 letters. In some embodiments, the method reduces inflammation in the eye of the subject. In some embodiments, the method results in a longer period of time between VEGF modulator injections.

In some embodiments, the non-surgical administration of TA according to the methods described herein reduces the number and/or frequency of administration of a VEGF modulator to the subject. Thus, in some embodiments, the administration of the triamcinolone increases the effectiveness and/or durability of the VEGF modulator treatment. For example, in some embodiments, the SCS administration of triamcinolone results in a need for fewer administrations of a VEGF modulator, and/or results in a longer period of time between administrations of a VEGF modulator.

In some embodiments, the non-surgical administration of TA according to the methods described herein results in maintenance of the improved BCVA and/or the improved CST in the subject for at least 12 weeks, at least 16 weeks, at least 20 weeks, at least 24 weeks, at least 30 weeks, at least 36 weeks, at least 40 weeks, at least 44 weeks, at least 48 weeks, or longer after the initial dose of TA. In some embodiments, the non-surgical administration of TA according to the methods described herein results in maintenance of the improved BCVA and/or the improved CST in the subject for at least 12 weeks, at least 16 weeks, at least 20 weeks, at least 24 weeks, at least 30 weeks, at least 36 weeks, at least 40 weeks, at least 44 weeks, at least 48 weeks, or longer after a second dose of TA. In further embodiments, the second dose of TA is administered about 12 weeks after the first dose of TA.

### BRIEF DESCRIPTION OF THE DRAWINGS

FIG. 1 is a cross-sectional view of an illustration of the human eye.

FIG. 2 is a cross-sectional view of a portion of the human eye of FIG. 1 taken along the line 2-2.

FIGS. 3 and 4 are cross-sectional views of a portion of the human eye of FIG. 1 taken along the line 3-3, illustrating the suprachoroidal space without and with, respectively, the presence of a fluid.

FIG. 5 is a perspective view of a medical injector according to an embodiment.

FIG. 6 is a partially exploded view of the medical injector of FIG. 5.

FIG. 7 is an exploded view of the medical injector of FIG. 5, shown without a needle cap.

FIG. 8 is a front view of a handle included in the medical injector of FIG. 5.

FIG. 9 is a cross-sectional view of the handle of FIG. 8 taken along the line 9-9.

FIG. 10 is a perspective view of a barrel included in the medical injector of FIG. 5.

FIG. 11 is an exploded view of a needle hub included in the medical injector of FIG. 5.

FIG. 12 is a front view of the needle hub of FIG. 9.

FIG. 13 is an enlarged view of a portion of the needle hub of FIG. 12, identified by the region Z₁.

FIG. 14 is a rear perspective view of a needle cap included in the medical injector of FIG. 5.

FIG. 15 is a front view of the medical injector of FIG. 5.

FIG. 16 is a cross-sectional view of the medical injector of FIG. 5, taken along the like 16-16 in FIG. 15.

FIG. 17 is a view of the medical injector of FIG. 5 in use during an injection procedure into the human eye.

FIG. 18 is an enlarged view of a portion of the medical injector of FIG. 5 and the human eye, identified in FIG. 17 by the region Z₂.

FIG. 19 is an exploded view of a needle hub configured for use with the medical injector of FIG. 5, according to an embodiment.

FIG. 20 is a front view of the needle hub of FIG. 19.

FIG. 21 is a flowchart illustrating a method of using a medical injector to inject a medicament into an eye.

FIG. 22 is schematic diagram of a clinical study to assess the efficacy of 2 SCS doses of CLS-TA.

FIG. 23 shows key inclusion and exclusion criteria for the phase 3 clinical study.

FIG. 24 shows the patient disposition of the phase 3 clinical study. Subjects were randomized 3:2 in the CLS-TA to sham (control) arms.

FIG. 25 is a bar graph showing the proportion of patients in each arm (CLS-TA treatment group vs. sham procedure control) that gained at least 15 ETDRS letters in BCVA from baseline, as measured at week 24 of the study. The p-value is based on a CMH Cochran-Mantel-Haenszel test for general association between treatment and response with stratification by country.

FIG. 26 shows the primary endpoint details in the phase 3 clinical study.

FIG. 27 is a bar graph showing the mean change (from baseline) at week 24 in BCVA, measured in ETDRS letters read, in each arm of the study (CLS-TA treatment group vs. sham procedure control).

FIG. 28 is a line graph showing the mean change (from baseline) at each visit in BCVA, measured in ETDRS letters read.

FIG. 29 is a bar graph showing the mean change in central retinal thickness (CST) in each arm (CLS-TA treatment group vs. sham procedure control) at week 24 of the study.

FIG. 30 is a line graph showing the change in CST at each visit of the study.

FIG. 31 is a bar graph showing the percent of subjects having 20% or greater improvement in CST at each week of the study.

FIG. 32 is a bar graph showing the percent of subjects who exhibited resolution of CST at each week of the study.

FIG. 33A is a bar graph showing the mean change from baseline in BCVA by anatomic location in patients in the CLS-TA arm (left bar at each of the 4 indicated anatomic location) or control arm (right bar at each of the 4 indicated anatomic locations).

FIG. 33B is a bar graph showing the mean change from baseline in CST by anatomic location in patients in the CLS-TA arm (left bar at each of the 4 indicated anatomic location) or control arm (right bar at each of the 4 indicated anatomic locations).

FIG. 34 is a bar graph showing the percent of subjects exhibiting a score of zero for three different types of inflammation: anterior chamber flare, anterior chamber cells, and vitreous haze.

FIG. 35 is a schematic diagram of the extension study to assess durability of the safety and efficacy of 2 SCS doses of CLS-TA.

FIG. 36 is a line graph showing the time in days to first rescue for the treatment of FIG. 37 is a line graph showing the mean and mean change in BCVA from baseline in the subjects in the initial 24 week study and the 24 week extension study.

FIG. 38 is a line graph showing the mean and mean change in CST from baseline in the subjects in the initial 24 week study and the 24 week extension study.

FIG. 39 is a bar graph showing the average number of treatment visits through week 24 in patients that received SCS injections of CLS-TA in combination with IVT injection of aflibercept, compared to IVT aflibercept monotherapy, for the treatment of DME.

### DETAILED DESCRIPTION OF THE INVENTION

Methods, devices and drug formulations are provided herein for treating posterior ocular disorders, for example diabetic macular edema (DME) or uveitis (e.g., infectious or non-infectious uveitis) and macular edema associated with uveitis. In one embodiment, the uveitis is intermediate, anterior, posterior or pan uveitis. In some embodiments, the drug formulation comprises triamcinolone. In some embodiments, the drug formulation comprises triamcinolone acetonide (TA). In some embodiments, the TA drug formulation is referred to herein as “CLS-TA.” In some embodiments, the methods comprise administering to the subject a dose of about 4 mg TA via injection in to the suprachoroidal space (SCS) of the eye of the subject. In some embodiments, the methods comprise administering to the subject two doses of about 4 mg TA via injection into the SCS of the eye of the subject. In further embodiments, the two doses are administered about 12 weeks apart.

Intravitreal injections result in drugs diffusing throughout the eye, including into the lens, iris and ciliary body at the front of the eye, which for some drugs, has been associated with safety issues, such as cataracts and elevated intraocular pressure (IOP) levels. Specifically, intravitreal administration of triamcinolone (TA) has been associated with cataracts and increases in IOP levels in 20% to 60% of patients. Because SCS injection of drugs appears to result in drug remaining localized in the retina and choroid without substantial diffusion to the vitreous or the front portion of the eye, without wishing to be bound by theory, it is thought that SCS injection has the potential to reduce the incidence of these side effects.

Current treatments for ocular diseases often require intravitreal injections of anti-inflammatory or other drugs. However ocular disorders often affect the posterior segment of the eye (e.g., choroid and retina) and therefore, specific targeting of these tissues might be more beneficial in modulating disease progression. The present invention addresses this need.

The methods and devices provided herein, for example, for the treatment of DME or macular edema associated with uveitis, such as non-infectious uveitis. The compositions and methods provided herein, in one embodiment, are used to restore or improve visual function. Without wishing to be bound by theory, in some embodiments the methods provided herein reduce macular edema affecting the retina, the tissue that lines the inside of the eye and is the part of the eye primarily responsible for vision, and the choroid, the layer adjacent to the retina that supplies the retina with blood, oxygen and nourishment. Macular edema is the build-up of fluid that can cause abnormal swelling of the macula, the portion of the retina responsible for central vision and color perception. This swelling can rapidly result in deterioration of vision and can eventually lead to blindness.

As used herein, “non-surgical” ocular drug delivery devices and methods refer to methods and devices for drug delivery that do not require general anesthesia and/or retrobulbar anesthesia (also referred to as a retrobulbar block). Alternatively or additionally, a “non-surgical” ocular drug delivery method is performed with an instrument having a diameter of 28 gauge or smaller. Alternatively or additionally, “non-surgical” ocular drug delivery methods do not require a guidance mechanism that is typically required for ocular drug delivery via a shunt or cannula.

As used herein, “surgical” ocular drug delivery includes insertion of devices or administration of drugs by surgical means, for example, via incision to expose and provide access to regions of the eye including the posterior region, and/or via insertion of a stent, shunt, or cannula.

The surgical and non-surgical posterior ocular disorder treatment methods and devices described herein are particularly useful for the local delivery of drugs to the posterior region of the eye, for example the retinochoroidal tissue, macula, retinal pigment epithelium (RPE) and optic nerve in the posterior segment of the eye. In another embodiment, the non-surgical methods and microneedles provided herein can be used to target drug delivery to specific posterior ocular tissues or regions within the eye or in neighboring tissue. In one embodiment, the methods described herein deliver drug specifically to the sclera, the choroid, the Brach's membrane, the retinal pigment epithelium, the subretinal space, the retina, the macula, the optic disk, the optic nerve, the ciliary body, the trabecular meshwork, the aqueous humor, the vitreous humor, and/or other ocular tissue or neighboring tissue in the eye of a human subject in need of treatment. The methods and microneedles provided herein, in one embodiment, can be used to target drug delivery to specific posterior ocular tissues or regions within the eye or in neighboring tissue.

In one embodiment of the methods described herein, a patient in need of treatment is administered a drug, e.g., TA, to the suprachoroidal space of one or both eyes for at least one dosing session. Non-surgical administration, in one embodiment, is achieved by inserting a microneedle into one or both eyes of the patient, for example the sclera, and injecting or infusing a drug formulation through the inserted microneedle and into the suprachoroidal space of the eye. Surgical administration, in another embodiment, is achieved by making a conjunctival peritomy in the eye to expose and provide access to a posterior region of the eye; or by any other traditional surgical means of accessing the posterior region of the eye, known in the art. In some embodiments, the treatment is administered via a shunt, stent, or cannula that is surgically placed into the eye of the subject.

In one embodiment, the effective amount of the drug administered to the SCS provides higher therapeutic efficacy of the drug, compared to the therapeutic efficacy of the drug when the identical dosage is administered intravitreally, topically, intracamerally, parenterally or orally. In one embodiment, the microneedle drug delivery methods described herein precisely deliver the drug into the SCS for subsequent local delivery to nearby posterior ocular tissues (e.g., the retina and choroid) in need of treatment. The drug may be released into the ocular tissues from the infused volume (or, e.g., from microparticles or nanoparticles in the drug formulation) for an extended period, e.g., several hours or days or weeks or months, after the non-surgical drug administration has been completed. This beneficially can provide increased bioavailability of the drug relative, for example, to delivery by topical application of the drug formulation to ocular tissue surfaces, or increased bioavailability compared to oral, parenteral on intravitreal administration of the same drug dosage. In some embodiments, the drug formulation includes TA.

With the methods and microneedle devices described herein, the SCS drug delivery methods advantageously include precise control of the depth of insertion into the ocular tissue, so that the microneedle tip can be placed into the eye so that the drug formulation flows into the suprachoroidal space and into one or more posterior ocular tissues surrounding the SCS, e.g., the choroid and retina. In one embodiment, insertion of the microneedle is in the sclera of the eye. In one embodiment, drug flow into the SCS is accomplished without contacting underlying tissues with the microneedle, such as choroid and retina tissues.

The methods provided herein, in one embodiment, achieve delivery of drug to the suprachoroidal space, thereby allowing drug access to posterior ocular tissues (e.g., the choroid and retina) not obtainable via topical, parenteral, intracameral or intravitreal drug delivery. Because the methods provided herein deliver drug to the posterior ocular tissue for the treatment of a posterior ocular disorder, the suprachoroidal drug dose sufficient to achieve a therapeutic response and/or the frequency of dosing in a human subject treated with the methods provided herein is less than the intravitreal, topical, parenteral or oral drug dose or dosing schedule sufficient to elicit the same or substantially the same therapeutic response. In one embodiment, the SCS delivery methods described herein allow for decreased drug dose of the posterior ocular disorder treating drug, compared to the intravitreal, topical, intracameral parenteral or oral drug dose sufficient to elicit the same or substantially the same therapeutic response. In a further embodiment, the suprachoroidal drug dose sufficient to elicit a therapeutic response is 75% or less, or 50% or less, or 25% or less than the intravitreal, topical parenteral or oral drug dose sufficient to elicit a therapeutic response. The therapeutic response, in one embodiment, is a reduction in severity of a symptom/clinical manifestation of the posterior ocular disorder (DME, uveitis, macular edema associated with uveitis, macular edema associated with RVO, wet AMD, choroidal neovascularization (CNV), wet AMD associated with CNV) for which the patient is undergoing treatment, or a reduction in number of symptom(s)/clinical manifestation(s) of the posterior ocular disorder for which the patient is undergoing treatment. In some embodiments, the therapeutic response of SCS delivery methods provided herein includes the increased effectiveness and/or reduced number and/or reduced frequency of administration of other drugs to subject suffering from a posterior ocular disorder. For example, in some embodiments, the therapeutic response of the SCS delivery methods provided herein includes increased effectiveness and/or reduced number and/or reduced frequency of administration of a VEGF modulator drug.

The term “suprachoroidal space,” is used interchangeably with suprachoroidal, SCS, suprachoroid and suprachoroidia, and describes the potential space in the region of the eye disposed between the sclera and choroid. This region primarily is composed of closely packed layers of long pigmented processes derived from each of the two adjacent tissues; however, a space can develop in this region as a result of fluid or other material buildup in the suprachoroidal space and the adjacent tissues. The “supraciliary space,” as used herein, is encompassed by the SCS and refers to the most anterior portion of the SCS adjacent to the ciliary body, trabecular meshwork and limbus. Those skilled in the art will appreciate that the suprachoroidal space frequently is expanded by fluid buildup because of some disease state in the eye or as a result of some trauma or surgical intervention. In the present description, however, the fluid buildup is intentionally created by infusion of a drug formulation into the suprachoroid to create the suprachoroidal space (which is filled with drug formulation). Not wishing to be bound by theory, it is believed that the SCS region serves as a pathway for uveoscleral outflow (i.e., a natural process of the eye moving fluid from one region of the eye to the other through) and becomes a real space in instances of choroidal detachment from the sclera.

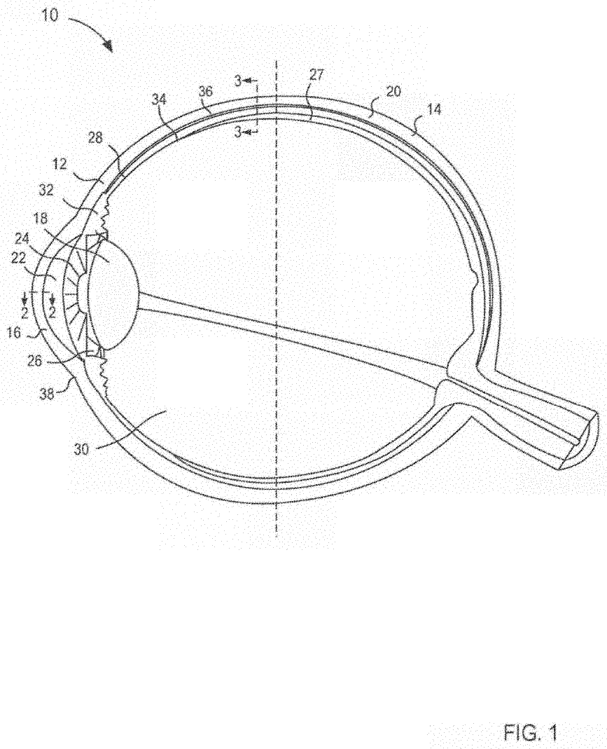

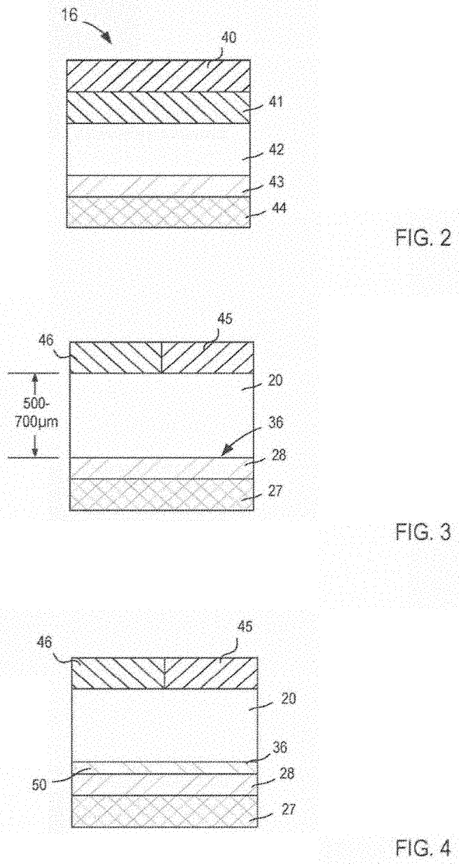

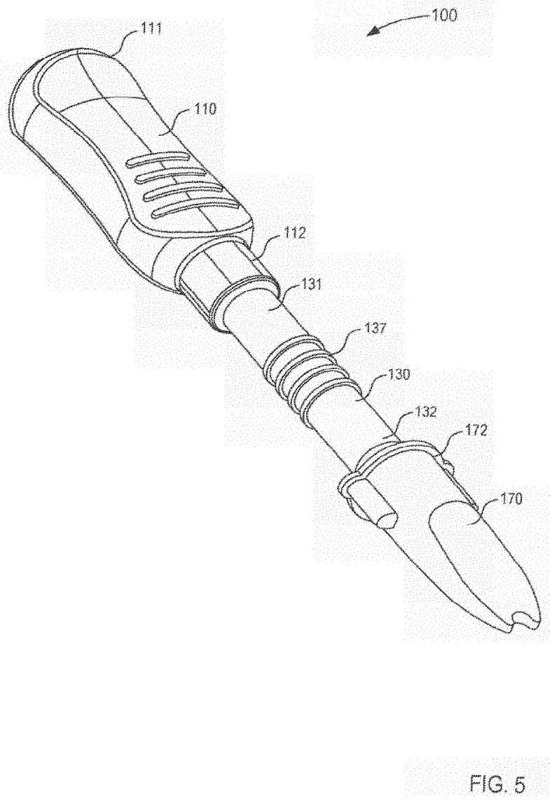

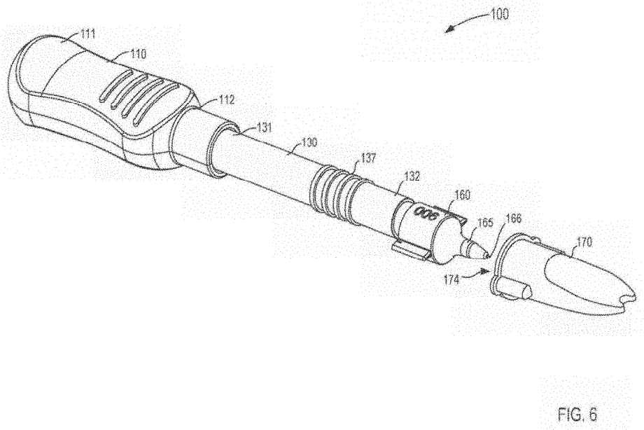

As used herein, “ocular tissue” and “eye” include both the anterior segment of the eye (i.e., the portion of the eye in front of the lens) and the posterior segment of the eye (i.e., the portion of the eye behind the lens). For reference, FIGS. 1-4 are a various views of a human eye 10 (with FIGS. 2-4 being cross-sectional views). While specific regions are identified, those skilled in the art will recognize that the proceeding identified regions do not constitute the entirety of the eye 10, rather the identified regions are presented as a simplified example suitable for the discussion of the embodiments herein. The eye 10 includes both an anterior segment 12 (the portion of the eye in front of and including the lens) and a posterior segment 14 (the portion of the eye behind the lens). The anterior segment 12 is bounded by the cornea 16 and the lens 18, while the posterior segment 14 is bounded by the sclera 20 and the lens 18. The anterior segment 12 is further subdivided into the anterior chamber 22, between the iris 24 and the cornea 16, and the posterior chamber 26, between the lens 18 and the iris 24. The cornea 16 and the sclera 20 collectively form a limbus 38 at the point at which they meet. The exposed portion of the sclera 20 on the anterior segment 12 of the eye is protected by a clear membrane referred to as the conjunctiva 45 (see e.g., FIGS. 2 and 3). Underlying the sclera 20 is the choroid 28 and the retina 27, collectively referred to as retinachoroidal tissue. A vitreous humour 30 (also referred to as the “vitreous”) is disposed between a ciliary body 32 (including a ciliary muscle and a ciliary process) and the retina 27. The anterior portion of the retina 27 forms an ora serrata 34. The loose connective tissue, or potential space, between the choroid 28 and the sclera 20 is referred to as the suprachoroid. FIG. 2 illustrates the cornea 16, which is composed of the epithelium 40, the Bowman's layer 41, the stroma 42, the Descemet's membrane 43, and the endothelium 44. FIG. 3 illustrates the sclera 20 with surrounding Tenon's Capsule 46 or conjunctiva 45, suprachoroidal space 36, choroid 28, and retina 27, substantially without fluid and/or tissue separation in the suprachoroidal space 36 (i.e., the in this configuration, the space is “potential” suprachoroidal space). As shown in FIG. 3, the sclera 20 has a thickness between about 500 μm and 700 μm. FIG. 4 illustrates the sclera 20 with the surrounding Tenon's Capsule 46 or the conjunctiva 45, suprachoroidal space 36, choroid 28, and retina 27, with fluid 50 in the suprachoroidal space 36.

The dashed line in FIG. 1 represents the equator of the eye 10. In some embodiments, the insertion site of any of the microneedles and/or methods described herein is between the equator and the limbus 38 (i.e., in the anterior portion 12 of the eye 10) For example, in some embodiments, the insertion site is between about two millimeters and 10 millimeters (mm) posterior to the limbus 38. In further embodiments, the injection site is about 4 to about 4.5 mm posterior to the limbus. In certain embodiments, the injection site is in the superior temporal quadrant. In other embodiments, the insertion site of the microneedle is at about the equator of the eye 10. In still other embodiments, the insertion site is posterior the equator of the eye 10. In this manner, a drug formulation can be introduced (e.g., via the microneedle) into the suprachoroidal space 36 at the site of the insertion and can flow through the suprachoroidal space 36 away from the site of insertion during an infusion event (e.g., during injection). In some embodiments, the drug formulation includes triamcinolone acetonide. In some embodiments, the drug formulation includes a VEGF modulator. In further embodiments, the VEG modulator may be aflibercept.

The microneedle may extend from the base of the microneedle device at any angle suitable for insertion into the eye 10. In a particular embodiment, the microneedle extends from the base at an angle of about 90 degrees to provide approximately perpendicular insertion of the microneedle into the surface of the eye. In another embodiment, the microneedle extends from the base at an angle from about 60 to about 110 degrees, from about 70 degrees to about 100 degrees, from about 80 degrees to about 90 degrees, or from about 85 degrees to about 95 degrees.

The microneedle device may comprise a means for controllably inserting, and optionally retracting, the microneedle into the ocular tissue. In addition, the microneedle device may include means of controlling the angle at which the at least one microneedle is inserted into the ocular tissue (e.g., by inserting the at least one microneedle into the surface of the ocular tissue at an angle of about 90 degrees).

In one embodiment, the depth of microneedle insertion into the ocular tissue can be controlled by the length of the microneedle, as well as other geometric features of the microneedle. For example, a flange or other a sudden change in microneedle width can be used to limit the depth of microneedle insertion. The microneedle insertion can also be controlled using a mechanical micropositioning system involving gears or other mechanical components that move the microneedle into the ocular tissue a controlled distance and, likewise, can be operated, for example, in reverse, to retract the microneedle a controlled distance. The depth of insertion can also be controlled by the velocity at which the microneedle is inserted into the ocular tissue. The retraction distance can be controlled by elastic recoil of the ocular tissue into which the microneedle is inserted or by including an elastic element within the microneedle device that pulls the microneedle back a specified distance after the force of insertion is released.

The angle of insertion can be directed by positioning the microneedle at a first angle relative to the microneedle base and positioning the base at a second angle relative to the ocular surface. In one embodiment, the first angle can be about 90° and the second angle can be about 0°. The angle of insertion can also be directed by having the microneedle protrude from a device housing through a channel in that housing that is oriented at a specified angle.

As provided throughout, in one embodiment, the methods described herein are carried out with a hollow or solid microneedle, for example, a rigid microneedle. As used herein, the term “microneedle” refers to a conduit body having a base, a shaft, and a tip end suitable for insertion into the sclera and other ocular tissue and has dimensions suitable for minimally invasive insertion and drug formulation infusion as described herein. Both the “length” and “effective length” of the microneedle encompass the length of the shaft of the microneedle and the bevel height of the microneedle. In some embodiments, the microneedle used to carry out the methods described herein comprises one of the devices disclosed in U.S. Pat. No. 9,539,139, issued Jan. 10, 2017 or International Patent Application Publication No. WO2014/179698 (Application No. PCT/US2014/036590), filed May 2, 2014 and entitled “Apparatus and Method for Ocular Injection,” each of which is incorporated by reference herein in its entirety for all purposes. In some embodiments, the microneedle used to carry out the methods described herein comprises one of the devices disclosed in International Patent Application Publication No. WO2014/036009 (Application No. PCT/US2013/056863), filed Aug. 27, 2013 and entitled “Apparatus and Method for Drug Delivery Using Microneedles,” incorporated by reference herein in its entirety for all purposes. In some embodiments, the microneedle is an SCS microinjector as described herein.

In some embodiments, features of the devices, formulations, and methods are provided in U.S. Pat. No. 9,636,332, U.S. Patent Application Publication No. 2018-0042765, International Patent Application Publication Nos. WO2014/074823 (Application No. PCT/US2013/069156), WO2015/195842 (Application No. PCT/US2015/036299), and/or WO2017/120601 (Application No. PCT/US2017/012757), each of which is hereby incorporated by reference in its entirety for all purposes.

In one embodiment, the device used to carry out one of the methods described herein comprises the device described in U.S. Design Patent Application Serial No. 29/506,275 entitled, “Medical Injector for Ocular Injection,” filed Oct. 14, 2014, the disclosure of which is incorporated herein by reference in its entirety for all purposes. In one embodiment, the device used to carry out one of the methods described herein comprises the device described in U.S. Patent Publication No. 2015/0051581 or U.S. Patent Publication No. 2017/0095339, which are each incorporated herein by reference in their entireties for all purposes. In some embodiments, such a device is an SCS microinjector as described herein.

In one embodiment, the microneedle is inserted into the eye of the human patient using a rotational/drilling technique and/or a vibrating action. In this way, the microneedle can be inserted to a desired depth by, for example, drilling the microneedles a desired number of rotations, which corresponds to a desired depth into the tissue. See, e.g., U.S. Patent Application Publication No. 2005/0137525, which is incorporated herein by reference, for a description of drilling microneedles. The rotational/drilling technique and/or a vibrating action may be applied during the insertion step, retraction step, or both.

As used herein, the words “proximal” and “distal” refer to the direction closer to and away from, respectively, an operator (e.g., surgeon, physician, nurse, technician, etc.) who would insert the medical device into the patient, with the tip-end (i.e., distal end) of the device inserted inside a patient's body first. Thus, for example, the end of a microneedle described herein first inserted inside the patient's body would be the distal end, while the opposite end of the microneedle (e.g., the end of the medical device being manipulated by the operator) would be the proximal end of the microneedle.

As used herein, the terms “about” and “approximately” generally mean plus or minus 10% of the value stated. For example, about 0.5 would include 0.45 and 0.55, about 10 would include 9 to 11, about 1000 would include 900 to 1100.

The term “fluid-tight” is understood to encompass both a hermetic seal (i.e., a seal that is gas-impervious) as well as a seal that is only liquid-impervious. The term “substantially” when used in connection with “fluid-tight,” “gas-impervious,” and/or “liquid-impervious” is intended to convey that, while total fluid imperviousness is desirable, some minimal leakage due to manufacturing tolerances, or other practical considerations (such as, for example, the pressure applied to the seal and/or within the fluid), can occur even in a “substantially fluid-tight” seal. Thus, a “substantially fluid-tight” seal includes a seal that prevents the passage of a fluid (including gases, liquids and/or slurries) therethrough when the seal is maintained at a constant position and at fluid pressures of less than about 5 pounds per square inch gage (psig), less than about 10 psig, less than about 20 psig, less than about 30 psig, less than about 50 psig, less than about 75 psig, less than about 100 psig and all values in between. Similarly, a “substantially liquid-tight” seal includes a seal that prevents the passage of a liquid (e.g., a liquid medicament) therethrough when the seal is maintained at a constant position and is exposed to liquid pressures of less than about 5 psig, less than about 10 psig, less than about 20 psig, less than about 30 psig, less than about 50 psig, less than about 75 psig, less than about 100 psig and all values in between.

As used herein, the term “hollow” includes a single, straight bore through the center of the microneedle, as well as multiple bores, bores that follow complex paths through the microneedles, multiple entry and exit points from the bore(s), and intersecting or networks of bores. That is, a hollow microneedle has a structure that includes one or more continuous pathways from the base of the microneedle to an exit point (opening) in the shaft and/or tip portion of the microneedle distal to the base.

The microneedle device in one embodiment, comprises a fluid reservoir for containing the therapeutic formulation (e.g., drug or cell formulation), e.g., as a solution or suspension, and the drug reservoir (which can include any therapeutic formulation) being in operable communication with the bore of the microneedle at a location distal to the tip end of the microneedle. The fluid reservoir may be integral with the microneedle, integral with the elongated body, or separate from both the microneedle and elongated body.

The microneedle and/or any of the components included in the embodiments described herein is/are formed and/or constructed of any suitable biocompatible material or combination of materials, including metals, glasses, semi-conductor materials, ceramics, or polymers. Examples of suitable metals include pharmaceutical grade stainless steel, gold, titanium, nickel, iron, gold, tin, chromium, copper, and alloys thereof. The polymer can be biodegradable or non-biodegradable. Examples of suitable biocompatible, biodegradable polymers include polylactides, polyglycolides, polylactide-co-glycolides (PLGA), polyanhydrides, polyorthoesters, polyetheresters, polycaprolactones, polyesteramides, poly(butyric acid), poly(valeric acid), polyurethanes and copolymers and blends thereof. Representative non-biodegradable polymers include various thermoplastics or other polymeric structural materials known in the fabrication of medical devices. Examples include nylons, polyesters, polycarbonates, polyacrylates, polymers of ethylene-vinyl acetates and other acyl substituted cellulose acetates, non-degradable polyurethanes, polystyrenes, polyvinyl chloride, polyvinyl fluoride, poly(vinyl imidazole), chlorosulphonate polyolefins, polyethylene oxide, blends and copolymers thereof. Biodegradable microneedles can provide an increased level of safety compared to non-biodegradable ones, such that they are essentially harmless even if inadvertently broken off into the ocular tissue.

In one embodiment, the hollow microneedle provided herein is fabricated using a laser or similar optical energy source. In one example, a microcannula may be cut using a laser to represent the desired microneedle length. The laser may also be used to shape single or multiple tip openings. Single or multiple cuts may be performed on a single microncannula to shape the desired microneedle structure. In one example, the microcannula may be made of metal such as stainless steel and cut using a laser with a wavelength in the infrared region of the light spectrum (e.g., from about 0.7 to about 300 μm). Further refinement may be performed using metal electropolishing techniques familiar to those in the field. In another embodiment, the microneedle length and optional bevel is formed by a physical grinding process, which for example may include grinding a metal cannula against a moving abrasive surface. The fabrication process may further include precision grinding, micro-bead jet blasting and ultrasonic cleaning to form the shape of the desired precise tip of the microneedle.

Further details of possible manufacturing techniques are described, for example, in U.S. Patent Application Publication No. 2006/0086689, U.S. Patent Application Publication No. 2006/0084942, U.S. Patent Application Publication No. 2005/0209565, U.S. Patent Application Publication No. 2002/0082543, U.S. Pat. Nos. 6,334,856, 6,611,707, 6,743,211 and PCT/US2014/36590, filed May 2, 2014, all of which are incorporated herein by reference in their entireties for all purposes.

In some embodiments, an apparatus includes a medicament container, a piston assembly and a handle. The medicament container defines a lumen configured to contain a medicament. A distal end portion of the medicament container includes a coupling portion configured to be removably coupled to a needle assembly. A proximal end portion of the medicament container includes a flange and a longitudinal shoulder. A distal end portion of the piston assembly includes an elastomeric member movably disposed within the lumen of the medicament container. The handle is coupled to a proximal end portion of the piston assembly such movement of the handle produces movement of the elastomeric member within the medicament container. The proximal end portion of the medicament container is movably disposed within the handle. A portion of the handle is configured to contact the flange to limit proximal movement of the handle relative to the medicament container. The handle includes a protrusion configured to engage the longitudinal shoulder of the medicament container to limit rotation of the handle relative to the medicament container.

Any of the compositions described herein can be injected using any suitable injector of the types shown and described herein. Any of the methods described herein can be performed use any suitable injector of the types shown and described herein. In this manner, the benefits of targeted drug delivery via a non-surgical approach can be realized. For example, in some embodiments, an apparatus includes a medicament container, a needle assembly, and a piston assembly. The medicament container contains a dose of a medicament, such as, for example a drug or cellular therapeutic, e.g., a steroid formulation or a cell suspension (e.g., a stem cell suspension). The dose has a delivered volume of at least about 20 μL, at least about 50 uL, at least about 100 μL, at least about 200 μL or at least about 500 μL. In one embodiment, the amount of therapeutic formulation delivered into the suprachoroidal space from the devices described herein is from about 10 μL to about 200 μL, e.g., from about 50 μL to about 150 μL. In another embodiment, from about 10 μL to about 500 μL, e.g., from about 50 μL to about 250 μL, is non-surgically administered to the suprachoroidal space.

In some embodiments, an apparatus includes a medicament container, a needle assembly, and a piston assembly. The medicament container contains a dose of a medicament, such as, for example a steroidal composition such as a triamcinolone composition. The needle assembly is coupled to a distal end portion of the medicament container, and includes a contact surface and a needle. The contact surface is configured to contact a target surface of an eye, and can include a convex surface and/or a sealing portion, as described herein. The needle is coupled to the base. A distal end portion of the piston assembly includes an elastomeric member movably disposed within the medicament container. A proximal end portion of the piston assembly is configured to receive a force to move the elastomeric within the medicament container to deliver the dose of the medicament via the needle assembly. The needle assembly and the piston assembly being collectively configured to deliver the dose of the medicament into a suprachoroidal space of the eye such that a therapeutic response resulting from the dose is substantially equivalent to a therapeutic response resulting from the delivery of a corresponding dose of the medicament via any one of an intravitreal delivery method, a topical delivery method, a parenteral delivery method or an oral delivery method. An amount of the dose is less than about 75 percent of an amount of the corresponding dose.

Additionally or alternatively, the needle assembly and the piston assembly being collectively configured to deliver the dose of the medicament into a suprachoroidal space of the eye such that an intraocular Cmax resulting from the dose is greater, for example at least about 1.25×, 1.5× or 2× greater than an intraocular Cmax resulting from the delivery of a corresponding dose of the medicament via any one of an intravitreal delivery method, a topical delivery method, a parenteral delivery method or an oral delivery method.

In some embodiments, the SCS microinjector is the medical injector shown in FIGS. 5-19. FIGS. 5-18 illustrate a medical injector 100 configured to deliver a medicament to, for example, ocular tissue, according to an embodiment. The medical injector 100 can be used in conjunction with any of the methods and therapeutic formulations described herein. More specifically, the medical injector 100 (also referred to herein as “injector”) can have a size, shape, and/or configuration that is based at least in part on constraints and/or challenges associated with delivering a drug formulation into ocular tissue. For example, as described in further detail herein, medicament delivery into ocular tissue using conventional devices and/or needles can lead to incomplete delivery of a dose, reduction in efficacy of an injected medicament, seeding of undesirable cells, trauma, etc. Thus, the medical injector 100 can have a size and/or configuration that effectively deliver a medicament to a portion of the eye such as a posterior region thereof.

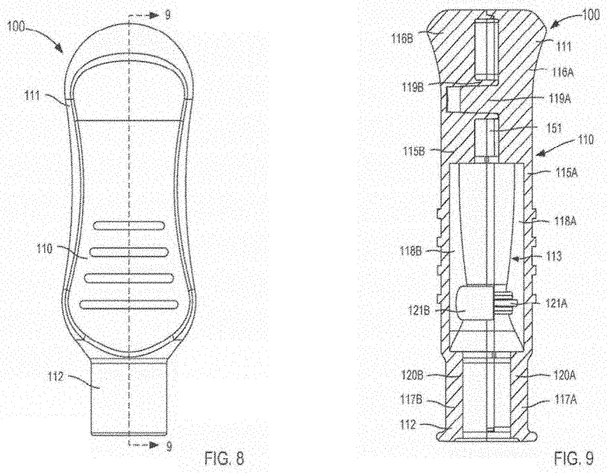

As shown, the medical injector 100 includes a handle 110, a barrel 130, a piston 150, a needle hub 160, and a cap 170. The handle 110 can be any suitable shape, size, and/or configuration. For example, in some embodiments, the handle 110 can have an ergonomic shape and/or size, which can enable to manipulate the injector 100 with one hand or with two hands. The handle 110 has a proximal end portion 111 and a distal end portion 112, and defines an inner volume 113 (see e.g., FIG. 9). The inner volume 113 of the handle 110 receives and/or is configured to house at least a portion of the barrel 130 and the piston 150, as described in further detail herein.

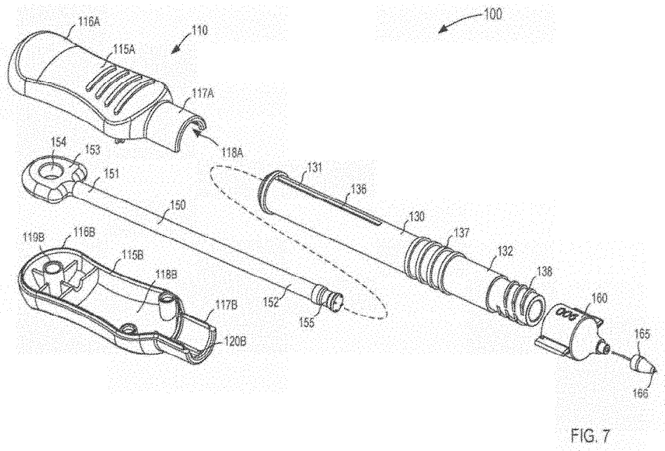

As shown in FIG. 7-9, the handle 110 is formed by coupling a first handle member 115A to a second handle member 115B. The handle member 115A and the handle member 115B can be relatively thin shelled or the like and can be formed from any suitable material such as the biocompatible materials described above. In other words, the handle members 115A and 1158B can be substantially hollow and/or can define an inner volume (e.g., the inner volume 113). The first handle member 115A has a proximal end portion 116A and a distal end portion 117A. Moreover, the first handle member 115A has an inner surface 118A that can include any suitable feature, cutout, coupler, wall, etc., any of which can be used to facilitate the coupling of the first handle member 115A to the second handle member 115B and/or to engage a portion of the piston 150 and/or the barrel 130. For example, as shown in FIG. 9, the inner surface 118A of the first handle member 115A can form a rib 120A, a retention member 119A, and at least one coupler 121A, which can be used, inter alia, to engage the barrel 130, the piston 150, and/or the second handle member 115B, respectively, as described in further detail herein.

Similarly, the second handle member 115B has a proximal end portion 116B and a distal end portion 117B. The second handle member 115B also has an inner surface 118B that forms a rib 120B, a retention member 119B, and at least one coupled 121B, which can be used to engage the barrel 130, the piston 150, and the first handle member 115A, respectively, as described in further detail herein. As shown in FIG. 9, for example, the first handle member 115A and the second handle member 115B are coupled together to collectively form the handle 100. The first handle member 115A and the second handle member 115B can be coupled in any suitable member. For example, in some embodiments, the retention member 119B of the second handle member 115B can define an opening or the like configured to matingly receive a portion of the retention member 119A of the first handle member 115A. Similarly, the at least one coupler 121B of the second handle member 119B can define an opening configured to matingly receive a portion of an associated coupler 121A of the first handle member 115A. In some embodiments, the retention member 119A and the coupler(s) 121B of the first handle member 115A can be configured to form a press or friction fit with an inner surface of the retention member 119B and the coupler(s) 121B of the second handle member 115B, which can be operable in coupling the first handle member 115A to the second handle member 115B. In other embodiments, the first handle member 115A and the second handle member 115B can be coupled via any suitable method such as, for example, an adhesive, an ultrasonic weld, a mechanical fastener, and/or the like. Furthermore, when the first handle member 115A is coupled to the second handle member 115B, the inner surfaces 118A and 118B of the handle members 115A and 115B, respectively, collectively define the inner volume 113 of the handle 110, as shown in FIG. 9.

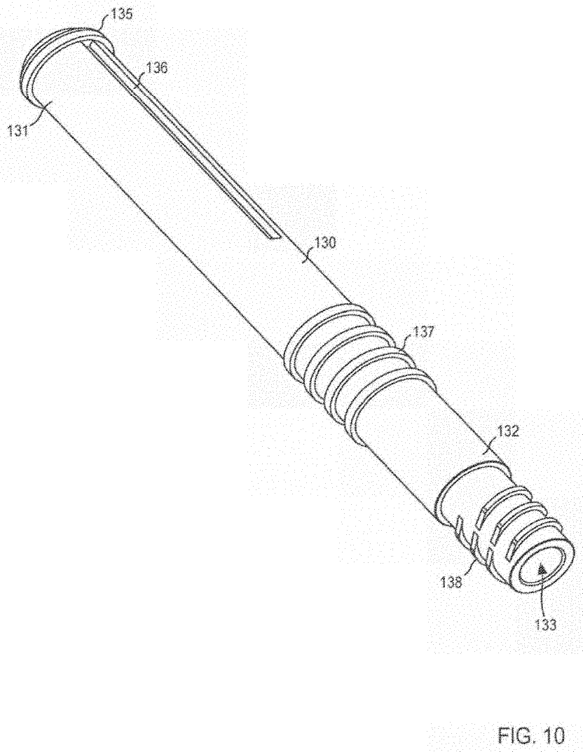

The barrel 130 of the injector 100 can be any suitable shape, size, or configuration. As shown in FIG. 10, the barrel 130 has a proximal end portion 131 and a distal end portion 132 and defines a lumen 133 therethrough. In addition, the barrel 130 has an outer surface that defines a set of slots 136 (only one is shown in FIG. 10) and a grip portion 137. The grip portion 137 can be configured to facilitate the use of the device by providing a user with a predetermined location to engage the injector 100. The grip portion 137 can have any suitable surface finish or the like, which can, in some instances, increase a friction between the grip portion 137 and a user's fingers and/or hand. In other embodiments, the barrel 130 does not include a grip portion.

The lumen 133 of the barrel 130 movably receives at least a portion of the piston 150, as described in further detail herein. Moreover, at least a portion of the lumen 133 can define a medicament volume configured to receive, store, house, and/or otherwise contain a medicament (e.g., a corticosteroid such as triamcinolone acetonide, or any other medicament described herein). In some embodiments, at least a portion of the barrel 130 can be substantially transparent and/or can include an indicator or the like configured to allow a user to visually inspect a volume of fluid (e.g., medicament/therapeutic formulation) within the lumen 133. In some instances, such an indicator can be, for example, any number of lines and/or markings associated with a volume of fluid disposed within the barrel 130. In other embodiments, the barrel 130 can be substantially opaque and/or does not include an indicator or the like.

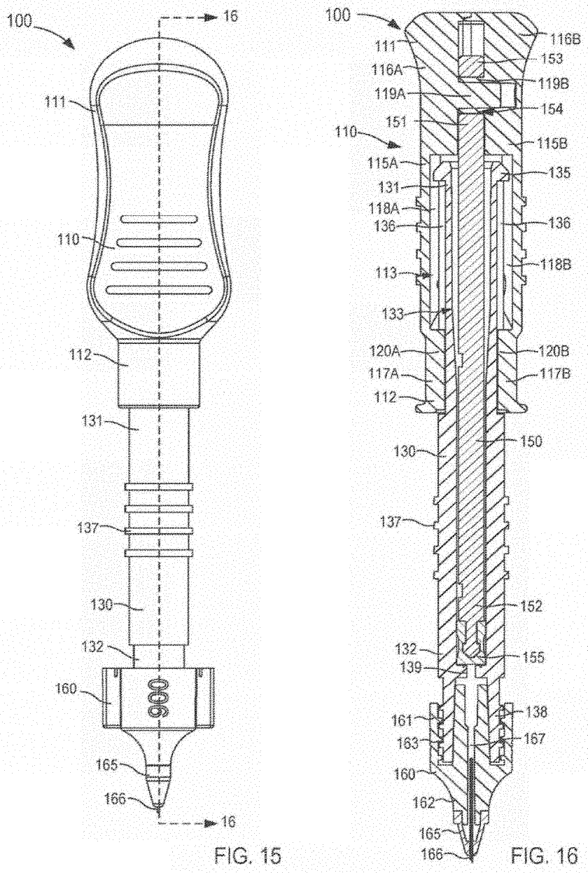

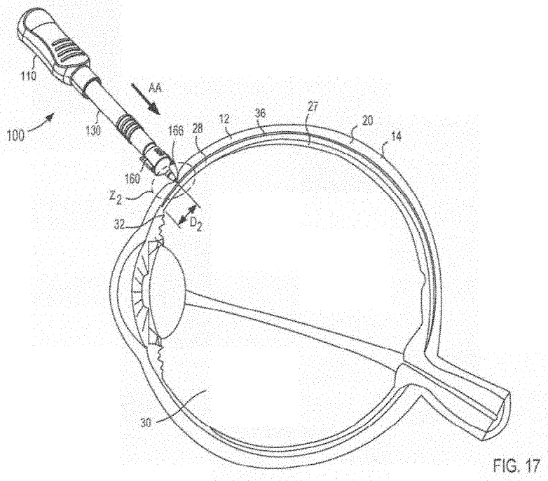

The distal end portion 132 includes and/or forms a coupler 138 configured to be physically and fluidically coupled to the needle hub 160, as described in further detail herein. The proximal end portion 131 of the barrel 130 includes a flanged end 135 and defines a set of slots 136 (only one slot is shown in FIG. 10). As described above, at least a portion of the barrel 130 is disposed within the inner volume 113 of the handle 110 (see, e.g., FIG. 16). Specifically, at least the proximal end portion 131 of the barrel 130 can be inserted into the handle 110 in such a manner that the handle 110 can be moved relative to the barrel 130. In other words, at least the proximal end portion 131 of the barrel 130 can be movably disposed within the inner volume 113 defined by the handle 110. Moreover, when the proximal end portion 131 of the barrel 130 is disposed in the handle 110, the ribs 120A and 120B of the handle members 115A and 115B, respectively, are movably disposed in its associated slot 136 defined by the barrel 130. Such an arrangement can, for example, define a range of motion of the handle 110 relative to the barrel 130. Such an arrangement can also limit a rotational motion of the handle 110 about the barrel 130 while allowing a translational motion of the handle 110 relative to the barrel 130 in a proximal or a distal direction. In this manner, during the injection operation, substantially all of the force applied by the user will urge the handle 110 (and therefore the piston 150) in the distal direction, and will not cause rotation of the piston 150 within the barrel 130. By limiting the rotational motion of the piston 150 (and particularly, the elastomeric member 155) within the barrel 130, the injection operation can be consistently performed. For example, by limiting the rotational motion of the elastomeric member 155 within the barrel 130, the force needed to overcome the static coefficient of friction between the elastomeric member 155 and the barrel 130 will be more consistent (between parts and/or injection) than if an applied force includes both translational (i.e., distal) and rotational components. This arrangement facilitates a more consistent “loss of resistance” felt at the handle 110 during an injection operation, as described below.

Additionally, the arrangement of the flanged end 135 of the barrel 130 and the inner surfaces 118A and 118B of the handle members 115A and 115B, respectively, can define a translational range of motion of the handle 110 relative to the barrel 130 in the proximal or the distal direction (see e.g., FIG. 16).

The piston 150 of the injector 100 can be any suitable shape, size, and/or configuration. For example, referring back to FIG. 7, the piston 150 can have a size and shape that are each associated with the handle 110 and/or the barrel 130, which in turn, can allow at least a portion of the piston 150 to be disposed within the handle 110 and/or the barrel 130. More specifically, the piston 150 has a proximal end portion 151 and a distal end portion 152. The proximal end portion 151 of the piston 150 is configured to be disposed within the inner volume 113 of the handle 110. As shown in FIG. 7, the proximal end portion 151 of the piston 150 includes a tab 153 or the like that defines an opening 154, which in turn, can receive at least a portion of the retention members 119A and 119B of the handle members 115A and 115B, respectively. For example, in some embodiments, during an assembly and/or manufacturing process and prior to coupling the handle members 115A and 115B, the proximal end portion 151 of the piston 150 can be positioned relative to the retention member 119B of the second handle member 115B such that at least a portion of the retention member 119B is disposed within the opening 154 defined by the piston 150. In other words, the tab 153 at or near the proximal end portion 151 of the piston 150 can be disposed about a portion of the retention member 119B prior to coupling the first handle member 115A to the second handle member 115B. As such, the piston 150 can be fixedly coupled to the handle 110.

The distal end portion 152 of the piston 150 is configured to be movably disposed in the lumen 133 of the barrel 130. As shown in FIG. 7, the distal end portion 152 of the piston 150 includes and/or is coupled to an elastomeric member 155. In some embodiments, the elastomeric member 155 can be monolithically formed with the piston 150 (e.g., overmolded or the like). In other embodiments, the elastomeric member 155 can be formed independently of the piston 150 and coupled thereto. The elastomeric member 155 can be made of an inert and/or biocompatible material, which can have any suitable hardness and/or durometer. For example, in some embodiments, the elastomeric member 155 can be formed from and/or constructed out of a rubber, silicone, plastic, nylon, polymers, any other suitable material or combination thereof. In some embodiments, at least a portion of the elastomeric member 155 can be configured to deform or the like while substantially maintaining its original shape. That is to say, the elastomeric member 155 can have a durometer that is sufficiently low to allow at least some deformation thereof, while preventing the elastomeric member 155 from being substantially reconfigured and/or the like.

The elastomeric member 155 can be disposed in the lumen 113 such that an outer surface of the elastomeric member 155 is in contact with an inner surface of the barrel 130 defining the lumen 133. In some embodiments, the elastomeric member 155 and the inner surface of the barrel 130 collectively form a substantially fluid-tight seal and/or a hermetic seal, which can, for example, prevent leakage, out gassing, contamination, and/or the like of a substance (e.g., a medicament) disposed within the barrel 130. Moreover, the elastomeric member 155 can have a size, shape and/or can be constructed from a material such that movement of the piston 150 and/or elastomeric member 155 within the barrel 130 is limited when a force applied is below a predetermined threshold. In this manner, the piston 150 can be maintained in a substantially fixed position relative to the barrel 130 until a force exerted, for example, on the handle 110 is sufficient to inject a medicament into a target tissue, as described in further detail herein. In some embodiments, the size, shape, and/or configuration of the elastomeric member 155 can be changed to, for example, increase or decrease an amount of force used to move the piston 150 within the barrel 130, which in some instances, can be based on one or more characteristics associated with a target tissue and/or the like, as described in further detail herein.

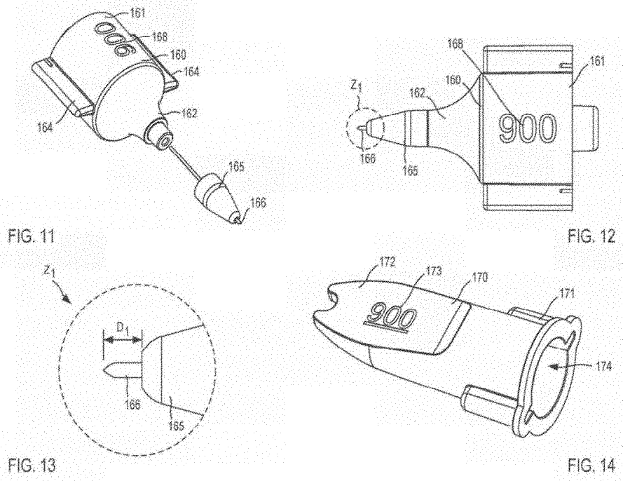

The needle hub 160 of the injector 100 can be any suitable shape, size, and/or configuration. As shown in FIGS. 11-13, 15, and 16, the needle hub 160 has a proximal end portion 161, a distal end portion 162, an indicator portion 168, and a pair of tabs 164, and defines a lumen 167 (see e.g., FIG. 16). The proximal end portion 161 of the needle hub 160 is configured to be coupled to the distal end portion 132 of the barrel 130. For example, the needle hub 160 can include a coupler 163 (see e.g., FIG. 16) that can matingly engage the coupler 138 of the barrel 130 to couple the needle hub 160 to the barrel 130 and to place the lumen 167 of the needle hub 160 in fluid communication with the lumen 133 of the barrel 130. In some embodiments, the coupler 163 of the needle hub 160 and the coupler 138 of the barrel 130 can form a threaded coupling or the like. In such embodiments, a user can, for example, engage the tabs 164 to rotate the needle hub 160 relative to the barrel 130, thereby threading the coupler 163 of the needle hub 160 onto the coupler 138 of the barrel 130. In some embodiments, the coupler 163 of the needle hub 160 can be a locking mechanism and/or the like such as, for example, a Luer-Lok® (or other locking mechanism) configured to form a fluid tight seal with the distal end portion 132 of the barrel 130 when coupled thereto. The distal end portion 162 of the needle hub 160 includes and/or is coupled to a base 165, which in turn, is coupled to and/or forms a microneedle 166, as described below. The indicator portion 168 of the needle hub 160 is configured to provide a visual indication associated with one or more characteristics of the microneedle 166. For example, in this embodiment, the indicator portion 168 can be configured to provide a visual indication associated with an effective length of the microneedle 166 (e.g., “900” micrometers, as shown in FIG. 12).

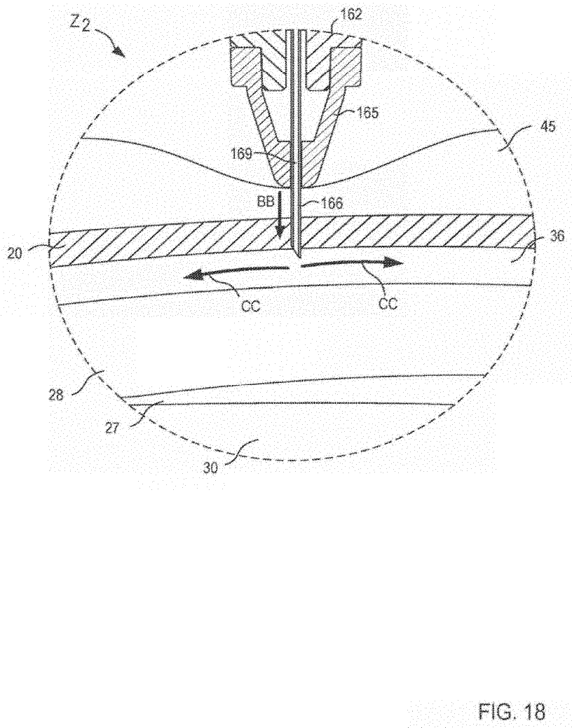

The base 165 can be any suitable shape, size, and/or configuration and can be configured to contact a portion of the ocular tissue during an injection event. For example, as shown, the base 165 has a convex distal end surface, which is configured to contact a target surface of a target tissue when a substance is conveyed through the needle into the target tissue (see, e.g., FIG. 18). In some embodiments, the distal end surface includes a sealing portion (not identified in the FIGS.) configured to define a substantially fluid-tight seal with the target surface when the distal end surface is in contact with the target surface. For example, the distal end surface of the base 165 can deform the target surface such that the sealing portion is contiguous with the target surface and forms the substantially fluid-tight seal. In some embodiments, the sealing portion can be symmetrical about the microneedle 166.

In some embodiments, the base 165 can be formed from a material or combination of materials that is/are relatively flexible and/or that has/have a relatively low durometer. In some instances, the base 165 can be formed from a material with a durometer that is sufficiently low to limit and/or prevent damage to the ocular tissue when placed in contact therewith. In some instances, the base 165 can be configured to deform (e.g., elastically or plastically) when placed in contact with the ocular tissue. In other embodiments, the base 165 can be formed from a material of sufficient hardness such that the target tissue (and not the base) is deformed when the base 165 is placed in contact with and/or pressed against the target tissue. In some embodiments, for example, the base 165 is constructed from a medical grade stainless steel, and has a surface finish of less than about 1.6 μm Ra. In this manner, the surface finish can facilitate the formation of a substantially fluid-tight seal between the base 165 and the target tissue.

Furthermore, when the base 165 is coupled to the needle hub 160, a lumen 169 defined by the microneedle 166 is in fluid communication with the lumen 167 of the needle hub 160 (see, e.g., FIG. 16). Thus, a substance can flow through the lumen 167 of the needle hub 160 and the lumen 169 of the microneedle 166 to be injected into a target tissue, as described in further detail herein.

The microneedle 166 can be any suitable device or structure that is configured to puncture a target tissue of a patient. For example, the microneedle 166 can be any of the microneedles described herein configured to puncture ocular tissue. In some embodiments, the microneedle 166 can be a 30 gauge microneedle, a 32 gauge microneedle or a 34 gauge microneedle. As shown in FIG. 13, the microneedle 166 extends from a distal surface of the base 165 by a distance D₁ (also referred to herein as an “effective length”). In some embodiments, the shape and/or size of the microneedle 166 can correspond with at least a portion of a target tissue. For example, in some embodiments, the effective length of the microneedle 166 (e.g., the portion of the microneedle 166 that is outside or distal to the base 165) can correspond with a portion of ocular tissue such that when the microneedle 166 is inserted into the ocular tissue, a portion of the microneedle 166 is disposed within the sclera or suprachoroidal space of the eye. Specifically, in this embodiment, the effective length and/or the distance D₁ is about 900 micrometers (μm). Moreover, the indicator portion 168 of the needle hub 160 can be configured to provide a user with a visual indication associated the effective length and/or distance D₁. Although not shown in FIGS. 11-13, in some embodiments, the microneedle 166 can have a bevel geometry (e.g., bevel angle, bevel height, bevel aspect ratio or the like), which can facilitate the piercing and/or insertion of a tip of the microneedle 166 into the target tissue and the opening (not shown) of the microneedle 166 can be maintained within a desired region during an injection event. In some embodiments, the microneedle 166 or any of the microneedles described herein can include a bevel or other characteristics of the types shown and described in International Patent Application Publication No. WO2014/036009 (Application No. PCT/US2013/056863), filed Aug. 27, 2013 and entitled “Apparatus and Method for Drug Delivery Using Microneedles” and/or International Patent Application Publication No. WO2014/179698 (International Application No. PCT/US2014/036590), filed May 2, 2014 and entitled “Apparatus and Method for Ocular Injection,” each of which is incorporated by reference herein in its entirety for all purposes.

As described above, the base 165 can be coupled to the needle hub 160, which in turn, is coupled to the barrel 130 such that the lumen 133 of the barrel, the lumen 167 of the needle hub 160, and the lumen 169 of the microneedle 166 define a fluid flow path through which a medicament and/or substance contained within the barrel 130 can flow, for example, to be injected into a target tissue.

The cap 170 of the injector 100 is removably disposed adjacent to a distal end portion 132 of the barrel 130 and is configured to substantially house, cover, enclose, protect, isolate, etc. at least a portion of the needle hub 160. More specifically, the cap 170 can be moved relative to the remaining portions of the medical injector 100 to position at least a portion of the needle hub 160 within an inner volume 174 (see, e.g., FIG. 14) of the cap 170. As such, the cap 170 can have a size and/or shape that is associated with and/or at least partially based on a size and/or shape of the needle hub 160. In some embodiments, the cap 170 and a portion of the needle hub 160 can collectively define a friction fit or the like, which can be operable in maintaining the cap 170 in a substantially fixed position relative to the needle hub 160. In addition, in some embodiments, the cap 170 and the portion of the needle hub 160 can collectively form a substantially fluid tight and/or substantially hermetic seal, which in turn, can maintain the sterility of a microneedle 166 prior to use of the medicament delivery device 100. For example, although not shown, the cap 170 can include a plug, a seal, a sterilization member (e.g., wipe, pad, etc.), and/or the like configured to maintain the sterility of the microneedle 166 prior to use. Moreover, as shown in FIG. 14, the cap 170 includes an indicator portion 173 that can provide a visual indication to a user associated with a size and/or effective length of the microneedle 166. In some embodiments, the indicator portion 173 can be substantially similar in form and function to the indicator portion 168 of the needle hub 160 and can be configured to provide substantially the same visual indication.

As shown in FIGS. 15-18, in some instances, a user (e.g., a doctor, technician, nurse, physician, ophthalmologist, etc.) can manipulate the injector 100 to deliver a drug formulation to the suprachoroidal space of an eye according to an embodiment. In some instances, prior to an injection event, the user can, for example, couple the distal end portion 132 of the barrel 130 to a fluid reservoir or the like and/or any suitable transfer device (not shown) to transfer a volume of a medicament and/or drug formulation into the lumen of the barrel 130. For example, in some embodiments, the distal end portion 132 of the barrel 130 can be physically and fluidically coupled to a transfer adapter and/or the like having a puncture member configured to puncture a fluid reservoir containing a drug formulation such as those described herein. Such transfer adapters can be similar to the adapter 21280 shown and described in International Patent Application Publication No. WO2014/179698 (Application No. PCT/US2014/036590), filed May 2, 2014 and entitled “Apparatus and Method for Ocular Injection,” incorporated by reference herein in its entirety for all purposes. As such, the puncture member places the transfer adapter in fluid communication with the fluid reservoir. With the transfer adapter physically and fluidically coupled to the barrel 130, the transfer adapter similarly places the lumen 133 of the barrel 130 in fluid communication with the fluid reservoir.

With the barrel 130 in fluid communication with the fluid reservoir (not shown), the user can manipulate the injector 100 by moving the handle 110 relative to the barrel 130 in the proximal direction, which in turn, moves the piston 150 disposed within the lumen 133 of the barrel 130 in the proximal direction. As such, a volume associated with a portion of the lumen 133 defined by the barrel 130 distal to the elastomeric member 155 of the piston 150 increases and a volume associated with a portion of the lumen 133 proximal to the elastomeric member 155 decreases. In some embodiments, the friction fit and/or fluidic seal defined between the elastomeric member 155 and the inner surface of the barrel 130 can be such that the proximal movement of the piston 150 (e.g., the increase in volume of the portion of the lumen 133 distal to the elastomeric member 155) produces a negative pressure differential within the portion of the lumen 133, which can be operable in drawing a volume of the medicament and/or the drug formulation from the fluid reservoir and into the portion of the lumen 133 distal to the elastomeric member 155 (e.g., a medicament volume). In some embodiments, a predetermined volume of the drug formulation can be drawn into the lumen 133 of the barrel 130. In other embodiments, the volume of the drug formulation drawn into the lumen 133 is not predetermined. With the desired amount of drug formulation contained in the barrel 130, the user can, for example, decouple the barrel 130 from the transfer adapter (not shown). Moreover, in some embodiments, the coupler 138 and/or the distal end portion 132 of the barrel 130 can include a self-sealing port and/or any other suitable port configured to fluidically isolate the lumen 133 of the barrel 130 from a volume outside of the barrel 130. Although described above as transferring a volume of the drug formation from the fluid reservoir and into the lumen 133 of the barrel 130, in other embodiments, the injector 100 can be prefilled during, for example, a manufacturing process and/or any other time prior to use.

In some instances, with the desired amount of the drug formulation contained in the barrel 130, the user can manipulate the injector 100 to couple the needle hub 160 (e.g., disposed within the cap 170 or not disposed within the cap 170) to the distal end portion 132 of the barrel 130, thereby placing the lumen 169 of the microneedle 166 in fluid communication with the lumen 133 of the barrel 130. With the needle hub 160 coupled to the barrel 130, the user can remove the cap 170 from the needle hub 160 if it is disposed thereabout. In other instances, the cap 170 can already be removed. As such, the user can position the injector 100 relative to the ocular tissue such that the microneedle 166 disposed at or near a desired injection site. In some instances, the injection site can be a predetermined distance from, for example, the limbus 32. For example, as shown in FIG. 17, the injection site can be a distance D2 from the limbus 32 that is about 1 mm, about 2 mm, about 3 mm, about 4 mm, about 5 mm, about 6 mm, about 7 mm, about 8 mm, about 9 mm, about 10 mm, or more. In other instances, an injection site can be relative to any suitable portion of the eye.

With the microneedle 166 at or near the desired injection site, the base 165 of the needle hub 160 can be pressed against a target surface of the eye 10 as the microneedle 166 is inserted into the target surface. As such, the base 165 of the needle hub 160 can deform, define an indent, and/or otherwise form a “dimple” in the target surface (e.g., the conjunctiva 45 of the eye 10, as shown in FIG. 18). The “dimple” can facilitate a desired transfer of the medicament from the barrel 130 to the target region via the microneedle 166. The base 165 of the needle hub 160, and thus the dimple, can be maintained in such a position throughout the procedure (e.g., the injection of medicament into a SCS 36). In this manner, the “dimple” (e.g., the interface between the distal surface of the base 165 and the surface of the target location) can limit and/or prevent seepage of the medicament from the target region during injection and post-injection, thereby promoting desirable transfer of the medicament to the target region (e.g., the SCS 36). As described above, in some embodiments, the distal (or contact) surface of the base 165 can include a sealing portion, which can be a convex surface, a surface having a smooth finish (e.g., with a surface finish of less than Ra=1.6 μm) or the like.

In addition, in some embodiments, the microneedle 166 is inserted substantially perpendicular or at an angle from about 800 to about 100°, into the eye 10, reaching the suprachoroidal space in a short penetration distance (e.g., about 1.1 mm, about 1 mm, about 0.9 mm, or less). This is in contrast to long conventional microneedles 166 or a cannula, which approach the suprachoroidal space at a steep angle, taking a longer penetration path through the sclera 20 and other ocular tissues, increasing the invasiveness of the method, the size of the microneedle track and consequently increasing the risk of infection and/or vascular rupture. With such long microneedles 166, the ability to precisely control insertion depth is diminished relative to the microneedle 166 approach described herein.

Once the distal end portion of the microneedle 166 is disposed within at least one of the SCS 36, a lower portion of the sclera 20, and/or an upper portion of the choroid 28 of the eye 10 (FIG. 18), the medicament can be conveyed from the barrel 130. More specifically, while maintaining the dimple at the conjunctiva 45, a user can exert a force on the handle 110 to begin an infusion event. In some instances, such as during insertion, the force exerted by a user on the handle 110 can be insufficient to move the piston 150 within the barrel 130 when the distal tip of the microneedle 166 is not disposed within the desired position (e.g., when the microneedle 166 is in the sclera 20 and not the SCS 36 of the eye 10). Said another way, the injector 100 can be configured to assist a user in delivering at least a portion of the drug formulation to the region, while be configured or “calibrated” to limit and/or prevent delivery to another, different region.

In some embodiments, the injector 100 can be configured to inform the user when the distal tip of the microneedle 166 is in the target region, for example, such that the drug formulation can be delivered to the target region with high confidence. For example, the injector 100 can be configured to limit movement of the piston 150 within the lumen 133 of the barrel 130 when the distal tip of the microneedle 166 is disposed within a region of the eye 10, which has a greater density, such as the sclera 20. In some instances, the injector 100 can limit movement of the piston 150 within the lumen 133 when the applied force is below a predetermined threshold such as about 6 Newtons (N). Conversely, the injector 100 can allow movement of the piston 150 within the barrel 130 when the distal tip of the microneedle 166 is disposed within the target location (e.g., a region having a lower density, such as the SCS 36) and when the force having the magnitude of less than about 6 N is exerted on the piston 150 and/or the handle 110. In this manner, the system can be configured or “calibrated” to provide feedback (e.g., tactile feedback) to a user to allow the user to deliver the drug formulation to a target region with high confidence. In some instances, the user can observe movement, or lack of movement, of the piston 150 within the barrel 130 to determine whether medicament has been conveyed to the eye. If the medicament has not been conveyed, the user can respond accordingly. For example, the user can re-align the system, relocate to a different injection site, and/or use a different sized microneedle 166 (e.g., a different microneedle 166 length).

By way of example, a user can manipulate the injector 100 to insert the microneedle 166 into the eye 10 at a desired injection site. In some instances, if the distal tip of the microneedle 166 is not disposed in the desired position and is, instead, disposed in the sclera 20, a force exerted by the user on the handle 110 can be insufficient to move the piston 150 within the barrel 130. For example, the sclera 20 can produce a backpressure that, in conjunction with the friction between the elastomeric member 155 and the inner surface of the barrel 130 and resistance to flow caused by the characteristics of the drug (e.g., viscosity, density or the like), overcomes the force exerted by the user, thereby preventing and/or limiting delivery of the drug formulation to the sclera 20. In other words, the injector 100 is specifically configured or “calibrated” such that the force is insufficient to convey the drug formulation to the sclera 20. Conversely, when the distal tip of the microneedle 166 is disposed in, for example, the SCS 36 of the eye 10, the same force exerted by the user can be sufficient to move the piston 150 within the barrel, based at least in part on anatomical differences and/or the differences in material properties between the sclera 20 and the SCS 36 (e.g., densities or the like). In other words, the force can be sufficient to overcome a backpressure produced by the SCS 36. In this manner, the injector 100 can be configured to ensure that the injection is initiated only when the distal tip of the microneedle 166 is in and/or near the SCS 36 such that the drug formulation (e.g., a medicament such as, for example, triamcinolone, or any other medicament known in the art and/or described herein) can be delivered only to that region. Moreover, the SCS 36 produces a first pressure that resists and/or opposes flow from the distal tip of the microneedle 166, and the sclera 20 produces a second pressure that resists and/or opposes flow from the distal tip of the microneedle 166, which is higher than the first pressure. In this manner, a user can be informed by a loss of resistance felt at the handle 110 when the distal tip of the microneedle 166 is transitioned from the sclera 20 to or near the SCS 36.

In some embodiments, the force exerted can be about 2 N, about 3 N, about 4 N, about 5 N, about 6 N or more and inclusive of all ranges therebetween. In some embodiments, the piston 150 and the barrel 130 can be collectively configured such that the force produces an injection pressure within the barrel 130 of between about 100 kPa and about 500 kPa. For example, in some embodiments, the injection pressure can be about 100 kPa, 110 kPa, 120 kPa, 130 kPa, 140 kPa, 150 kPa, 160 kPa, 170 kPa, 180 kPa, 190 kPa, 200 kPa, 220 kPa, 240 kPa, 260 kPa, 280 kPa, 300 kPa, 320 kPa, 340 kPa, 360 kPa, 380 kPa, 400 kPa, 420 kPa, 440 kPa, 460 kPa, or about 480 kPa, inclusive of all ranges and values therebetween. The injection pressure can be sufficient to overcome the backpressure produced by SCS 36, but insufficient to overcome the backpressure produced by the sclera 20. In some embodiments, the force can be varied depending on the diameter of the barrel 130 and/or the piston 150, the viscosity of the drug formulation, and/or the material of the barrel 130 and/or the piston 150. In this manner, regardless of the variations in the piston 150, the barrel 130, and/or the drug formulation, the injector 100 produces an injection pressure within the barrel 130 of between about 100 kPa and about 500 kPa.

In some embodiments, the injector 100 can be configured such that injection distance traversed by the piston 150 is sufficient to deliver substantially the entire desired dose of the drug formulation into the SCS 36. In other embodiments, the injector 100 can be configured such that the injection distance traversed by the piston 150 is sufficient to deliver only a portion of the desired dose of the drug formulation into the SCS 36. In such embodiments, the injector 100 can be configured to initiate delivery of the drug formulation into the SCS 36, for example, to inform the user that the distal tip of the microneedle 166 is disposed within the SCS 36 (e.g., the user would see or otherwise detect that the piston 150 has moved, thus indicating the desired positioning of the microneedle 166). Said another way, the injector 100 can assist the user in determining whether the distal tip of the microneedle 166 is within the SCS 36 or not by initiating delivery of the drug formulation. In such embodiments, the injection distance can be a first injection distance. The user can then move the distal end portion of the piston 150 a second injection distance, for example, by applying a manual force on the piston 150 (e.g., by moving the handle 110 relative to the barrel 130, as described herein).

In some embodiments, the drug is injected over several seconds. For example, in some embodiments, the drug is injected over about 2-30 seconds, or over about 3-25 seconds, or over about 4-20 seconds, or over about 5-10 seconds.

After desirable conveyance of the medicament from the medicament container, the hub 160 can be maintained in contact with the target surface for a time to allow for a desired medicament absorption by the eye. In this manner, the medicament can spread through tissues of the back of the eye without the medicament seeping from the injection site (e.g., where the microneedle 166 pierced the conjunctiva). As described above, in some embodiments, the distal end surface of the base 165 can include a sealing portion configured to form a substantially fluid-tight seal with the conjunctiva to limit movement of the medicament out of the eye along the needle track. In this manner, the injector 100 and the methods described herein can facilitate delivery of the desired dose to the desired regions of the eye.

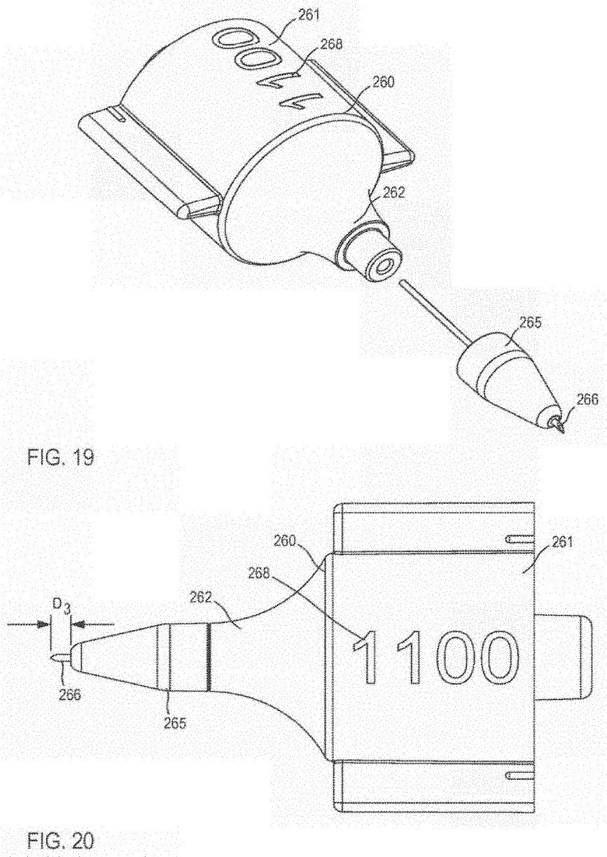

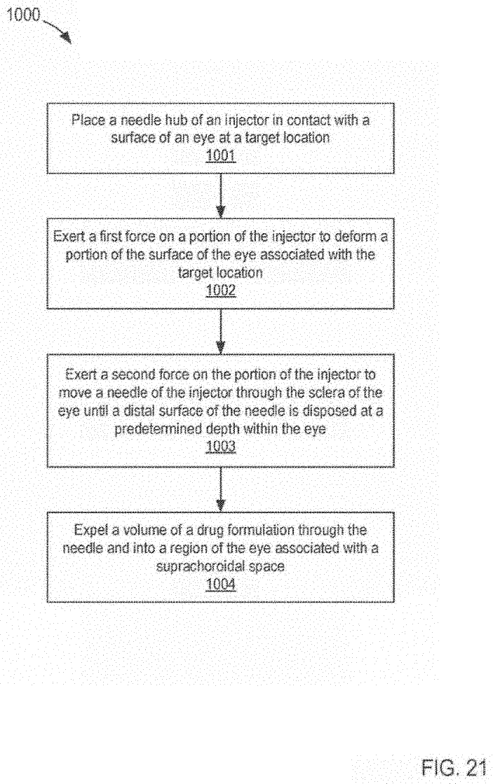

Although the microneedle 166 is described above as having an effective length that is about 900 μm, in other embodiments, the injector 100 can be coupled to a needle hub that includes a microneedle with any suitable effective length. For example, FIGS. 19 and 20 illustrate a needle hub 260 according to another embodiment. The needle hub 260 has a proximal end portion 261, a distal end portion 262, and an indicator portion 268. In some embodiments, the needle hub 260 can be substantially similar in form and function as the needle hub 160 described in detail above with reference to FIGS. 11-13. Thus, portions of the needle hub 260 are not described in further detail herein. The needle hub 260 can differ, however, by being coupled to a base 265 including a microneedle 266 with an effective length greater than the effective length of the microneedle 160. For example, in this embodiment, the microneedle 266 extends from the base 265 by a distance D₃ of about 1100 μm. Moreover, the indicator portion 268 of the needle hub 260 is configured to present a visual indication associated with the effective length and/or distance D₃ (e.g., represented in FIGS. 19 and 20 with the text “1100”).

In yet other embodiments, an injector can include a microneedle having an effective length of between about 200 μm and about 1500 μm. A short effective length microneedle (e.g., a length of between about 200 μm and about 400 μm) can be used, for example, in various subdermal injection procedures. Injectors with a longer effective length microneedle (e.g., a length of between about 1200 μm and about 1500 μm) can be used, for example, in various ocular procedures, such as, injection into the subretinal space. In some embodiments, the microneedle has an effective length of about 900 μm. In some embodiments, the microneedle has an effective length of about 1100 μm.

Referring now to FIG. 21, a flowchart is shown illustrating a method 1000 of using a medical injector to deliver a drug formulation to ocular tissue according to an embodiment. A first force is exerted on a portion of the injector to deform a portion of the surface of the eye associated with the target location, at 1002. A second force is exerted on the portion of the injector to move a needle (e.g., the microneedle) of the injector through the sclera of the eye until a distal surface of the needle is disposed at a predetermined depth within the eye, at 1003. A volume of a drug formulation is expelled through the needle and into a region of the eye associated with a suprachoroidal space, at 1004. While the method 1000 is described above as including a set of steps, in some instances, the method 1000 can include any number of optional steps and/or pre-procedural or post-procedural steps. The method 1000 and/or additional or alternative steps are described, for example, in U.S. Patent Application Publication No. 2018-0042765 and International Patent Application Publication No. WO2015/195842 (Application No. PCT/US2015/036299).

Although the medical injectors and methods described herein are shown as including a device including a needle and a reservoir including a medicament, in other embodiments, a medical device or kit can include a simulated medicament injector. In some embodiments, the simulated medicament injector can correspond to an actual medicament injector (e.g., the medical injector 100 described above) and can be used, for example, to train a user in the operation of the corresponding actual medical injector, to perform a “sham” injection as part of a clinical trial protocol, or the like.

A simulated medical injector can simulate the actual medical injector in any number of ways. For example, in some embodiments, the simulated medical injector can have a shape corresponding to a shape of the actual medical injector (e.g., injector 100), a size corresponding to a size of the actual medical injector (e.g., injector 100) and/or a weight corresponding to a weight of the actual medical injector (e.g., injector 100). Moreover, in some embodiments, the simulated medical injector can include components that correspond to the components of the actual medical injector. In this manner, the simulated medical injector can simulate the look, feel and sounds of the actual medical injector. For example, in some embodiments, the simulated medical injector can include external components (e.g., a base, a handle, or the like) that correspond to external components of the actual medical injector. In some embodiments, the simulated medical injector can include internal components (e.g., a plunger) that correspond to internal components of the actual medical injector.

In some embodiments, however, the simulated medical injector can be devoid of a medicament and/or those components that cause the medicament to be delivered (e.g., a microneedle). In this manner, the simulated medical injector can be used to train a user in the use of the actual medical injector without exposing the user to a needle and/or a medicament. Moreover, the simulated medical injector can have features to identify it as a training device to prevent a user from mistakenly believing that the simulated medical injector can be used to deliver a medicament.

The microneedle devices described herein also may be adapted to use the one or more microneedles as a sensor to detect analytes, electrical activity, and optical or other signals. The sensor may include sensors of pressure, temperature, chemicals, and/or electromagnetic fields (e.g., light). Biosensors can be located on or within the microneedle, or inside a device in communication with the body tissue via the microneedle. The microneedle biosensor can be any of the four classes of principal transducers: potentiometric, amperometric, optical, and physiochemical. In one embodiment, a hollow microneedle is filled with a substance, such as a gel, that has a sensing functionality associated with it. In an application for sensing based on binding to a substrate or reaction mediated by an enzyme, the substrate or enzyme can be immobilized in the needle interior. In another embodiment, a wave guide can be incorporated into the microneedle device to direct light to a specific location, or for detection, for example, using means such as a pH dye for color evaluation. Similarly, heat, electricity, light, ultrasound or other energy forms may be precisely transmitted to directly stimulate, damage, or heal a specific tissue or for diagnostic purposes.

The microneedle device for non-surgically delivering drug to the suprachoroidal space of the eye of a human subject, in one embodiment, comprises a hollow microneedle. The device may include an elongated housing for holding the proximal end of the microneedle. The device may further include a means for conducting a drug formulation through the microneedle. For example, the means may be a flexible or rigid conduit in fluid connection with the base or proximal end of the microneedle. The means may also include a pump or other devices for creating a pressure gradient for inducing fluid flow through the device. The conduit may in operable connection with a source of the drug formulation. The source may be any suitable container. In one embodiment, the source may be in the form of a conventional syringe. The source may be a disposable unit dose container.

The transport of drug formulation or biological fluid through a hollow microneedle can be controlled or monitored using, for example, one or more valves, pumps, sensors, actuators, and microprocessors. For instance, in one embodiment the microneedle device may include a micropump, microvalve, and positioner, with a microprocessor programmed to control a pump or valve to control the rate of delivery of a drug formulation through the microneedle and into the ocular tissue. The flow through a microneedle may be driven by diffusion, capillary action, a mechanical pump, electroosmosis, electrophoresis, convection or other driving forces. Devices and microneedle designs can be tailored using known pumps and other devices to utilize these drivers. In one embodiment, the microneedle device may further include an iontophoretic apparatus, similar to that described in U.S. Pat. No. 6,319,240 to Beck, for enhancing the delivery of the drug formulation to the ocular tissue. In another embodiment the microneedle devices can further include a flowmeter or other means to monitor flow through the microneedles and to coordinate use of the pumps and valves.

In some embodiments, the flow of drug formulation or biological fluid can be regulated using various valves or gates known in the art. The valve may be one which can be selectively and repeatedly opened and closed, or it may be a single-use type, such as a fracturable barrier. Other valves or gates used in the microneedle devices can be activated thermally, electrochemically, mechanically, or magnetically to selectively initiate, modulate, or stop the flow of material through the microneedles. In one embodiment, the flow is controlled with a rate-limiting membrane acting as the valve.

In other embodiments, the flow of drug formulation or biological fluid can be regulated by the internal friction of various components, the characteristics of the medicament to be injected (e.g., the viscosity) and/or the characteristics of the desired injection site. For example, as described above, in some embodiments, a drug product can be configured for delivery of a specific formulation to a specific location. In such embodiments, a drug product can include a microinjector (e.g., microinjector 100) and a medicament (e.g., triamcinolone or any other formulations described herein) that is configured to deliver the medicament to a specific target region (e.g., the SCS). In this example, the drug product can be configured such that the flow of the medicament is limited when injection is attempted into a different target region having a higher density (e.g., the sclera). Thus, the drug product is configured to regulate the flow by allowing flow when the injection is attempted into the desired target region.

The microneedle, in one embodiment, is part of an array of two or more microneedles such that the method further includes inserting at least a second microneedle into the sclera without penetrating across the sclera. In one embodiment, where an array of two or more microneedles are inserted into the ocular tissue, the drug formulation of each of the two or more microneedles may be identical to or different from one another, in drug, formulation, volume/quantity of drug formulation, or a combination of these parameters. In one case, different types of drug formulations may be injected via the one or more microneedles. For example, inserting a second hollow microneedle comprising a second drug formulation into the ocular tissue will result in delivery of the second drug formulation into the ocular tissue.

In another embodiment, the device includes an array of two or more microneedles. For example, the device may include an array of from 2 to 1000 (e.g., from 2 to 100 or from 2 to 10) microneedles. In one embodiment, a device includes between 1 and 10 microneedles. An array of microneedles may include a mixture of different microneedles. For instance, an array may include microneedles having various lengths, base portion diameters, tip portion shapes, spacings between microneedles, drug coatings, etc. In embodiments wherein the microneedle device comprises an array of two or more microneedles, the angle at which a single microneedle extends from the base may be independent from the angle at which another microneedle in the array extends from the base.

The SCS drug delivery methods provided herein allow for the delivery of drug formulation over a larger tissue area and to more difficult to target tissue in a single administration as compared to previously known needle devices. Not wishing to be bound by theory, it is believed that upon entering the SCS the drug formulation flows circumferentially from the insertion site toward the retinochoroidal tissue, macula, and optic nerve in the posterior segment of the eye as well as anteriorly toward the uvea and ciliary body. In addition, a portion of the infused drug formulation may remain in the SCS as a depot, or remain in tissue overlying the SCS, for example the sclera, near the microneedle insertion site, serving as additional depot of the drug formulation that subsequently can diffuse into the SCS and into other adjacent posterior tissues.

The terms “subject” and “patient” are used interchangeably herein. The human subject treated with the methods and devices provided herein may be an adult or a child. In one embodiment, the patient presents with a retinal thickness of greater than 300 μm (e.g., central retinal thickness or central subfield thickness as measured by optical coherence tomography). In another embodiment, the patient in need of treatment has a BCVA score of ≥20 letters read in each eye (e.g., 20/400 Snellen approximate). In yet another embodiment, the patient in need of treatment has a BCVA score of ≥20 letters read in each eye (e.g., 20/400 Snellen approximate), but <70 letters read in the eye in need of treatment.

The patient in some embodiments has macular edema (ME). In some embodiments, the patient has diabetic macular edema (DME). The patient in one embodiment has macular edema (ME) that involves the fovea. In one embodiment, in a method for treating ME associated with uveitis, the ME is due to the uveitis and not due to any other cause. In an embodiment for treating ME following retinal vein occlusion (RVO), the ME is due to RVO and not due to any other cause of ME. In a further embodiment, the RVO is branch retinal vein occlusion (BRVO), hemiretinal vein occlusion (HRVO) or central retinal vein occlusion (CRVO). In one embodiment, the patient in need of treatment experiences a decrease in visual acuity due to the ME.

The microneedle devices and non-surgical methods described herein may be used to deliver drug formulations to the eye of a human subject, particularly for the treatment, diagnosis, or prevention of a posterior ocular disorder, such as uveitis (e.g., non-infectious, infectious, intermediate, anterior, posterior or pan uveitis), macular edema associated with uveitis, e.g., non-infectious, intermediate, anterior, posterior or pan uveitis and macular edema associated with RVO. In one embodiment, the drug formulation comprises an effective amount of an anti-inflammatory drug. In one embodiment, the patient is in need of treatment of macular edema associated with uveitis or macular edema associated with RVO and the drug formulation comprises an anti-inflammatory drug selected from a steroid compound and a non-steroidal anti-inflammatory drug (NSAID). In even a further embodiment, the drug formulation is a triamcinolone formulation, e.g., a triamcinolone acetonide formulation.

Posterior ocular disorders amenable for treatment by the methods, devices and drug formulations described herein can include, but are not limited to, uveitis (e.g., infectious uveitis, non-infectious uveitis, chronic uveitis, and/or acute uveitis), macular edema, diabetic macular edema (DME), macular edema associated with uveitis (encompassing macular edema associated with infectious uveitis and macular edema associated with non-infectious uveitis), macular edema following retinal vein occlusion (RVO), and/or macular edema associated with RVO. In some embodiments, the posterior ocular disorder is macular edema associated with uveitis. In a further embodiment, the uveitis is a non-infectious uveitis.

The uveitis can be either acute or chronic uveitis. Uveitis, and macular edema associated with uveitis can be caused by infectious causes leading to infectious uveitis, such as infection with viruses, fungi, parasites, and/or the like. Uveitis can also be caused by non-infectious causes, such as the presence of noninfectious foreign substances in the eye, autoimmune diseases, surgical and/or traumatic injury, and/or the like. Disorders caused by pathogenic organisms that can lead to infectious uveitis, and to macular edema associated with infectious uveitis, include, but are not limited to, toxoplasmosis, toxocariasis, histoplasmosis, herpes simplex or herpes zoster infection, tuberculosis, syphilis, sarcoidosis, Vogt-Koyanagi-Harada syndrome, Behcet's disease, idiopathic retinal vasculitis, Vogt-Koyanagi-Harada Syndrome, acute posterior multifocal placoid pigment epitheliopathy (APMPPE), presumed ocular histoplasmosis syndrome (POHS), birdshot chroidopathy, Multiple Sclerosis, sympathetic opthalmia, punctate inner choroidopathy, pars planitis, or iridocyclitis. Acute uveitis and/or macular edema associated with acute uveitis occurs suddenly and may last for up to about six weeks. In chronic uveitis and/or macular edema associated with chronic uveitis, the onset of signs and/or symptoms is gradual, and symptoms last longer than about six weeks. The uveitis can be of any anatomic subtype (anterior, intermediate, posterior, or panuveitis).

Signs of uveitis include ciliary injection, aqueous flare, the accumulation of cells visible on ophthalmic examination, such as aqueous cells, retrolental cells, and vitreouscells, keratic precipitates, and hypema. Symptoms of uveitis include pain (such as ciliary spasm), redness, photophobia, increased lacrimation, and decreased vision. Posterior uveitis affects the posterior or choroid part of the eye. Inflammation of the choroid part of the eye is also often referred to as choroiditis. Posterior uveitis is may also be associated with inflammation that occurs in the retina (retinitis) or in the blood vessels in the posterior segment of the eye (vasculitis). In one embodiment, the methods provided herein comprise non-surgically administering to a uveitis patient suffering from macular edema associated with uveitis (e.g., non-infectious uveitis) in need thereof, an effective amount of an anti-inflammatory drug formulation to the SCS of the eye of the patient. In a further embodiment, the patient experiences a reduction in the severity of the symptoms of with macular edema associated with uveitis, after administration of the drug formulation. In one embodiment, the drug is a steroidal compound. In even a further embodiment, the drug is triamcinolone. In some embodiments, the drug formulation includes aflibercept and is used to treat a patient for wet AMD, CNV, wet AMD associated with CNV or wet AMD associated with RVO. In some embodiments, the drug formulation includes triamcinolone acetonide and is used in conjunction with aflibercept to treat a patient for wet AMD, CNV, wet AMD associated with CNV or wet AMD associated with RVO; or is used to improve any VEGF therapy in ocular disease.

In one embodiment, the patient undergoing one of the treatment methods provided herein, for example, the treatment of macular edema associated with uveitis or macular edema associated with RVO, experiences a reduction in fluid accumulation, inflammation, neuroprotection, complement inhibition, drusen formation, scar formation, and/or a reduction in choriocapillaris or choroidal neocasvularization.

Without wishing to be bound by theory, upon non-surgical SCS administration, the drug remains localized in the posterior segment of the eye, specifically, the choroid and retina. Limiting drug exposure to other eye tissues, in one embodiment, reduces the incidences of side effects associated with the prior art methods.

In one embodiment, from about 2 to about 24 dosing sessions are employed, for example, from about 2 to about 24 intraocular dosing sessions (e.g., intravitreal or suprachoroidal injection). In a further embodiment, from about 3 to about 30, or from about 5 to about 30, or from about 7 to about 30, or from about 9 to about 30, or from about 10 to about 30, or from about 12 to about 30 or from about 12 to about 24 dosing sessions are employed. In particular embodiments, the methods provided herein comprise two dosing sessions, wherein 4 mg TA is administered via non-surgical administration to the SCS in each of the two dosing sessions. In some embodiments, the two dosing sessions are about 1 2, 3, 4, 5, 6, 7, 8, 9, 10, 11, 12, 13, 14, 15, 16, 17, 18, 19, 20, 21, 22, 23, or 24 weeks apart. In certain embodiments, the two dosing sessions are about 12 weeks apart.

Treatment regimens will vary based on the therapeutic formulation being delivered and/or the indication being treated. In one embodiment, a single dosing session is effective in treating one of the indications described herein. However, in another embodiment, multiple dosing sessions are employed. In one embodiment, where multiple dosing sessions are employed, the dosing sessions are spaced apart by from about 10 days to about 100 days, or from about 10 days to about 90 days, or from about 10 days to about 80 days, or from about 10 days to about 40 days, or from about 10 days to about 30 days, or from about 10 days to about 20 days. In another embodiment, where multiple dosing sessions are employed, the dosing sessions are spaced apart by from about 20 days to about 60 days, or from about 20 days to about 50 days, or from about 20 days to about 40 days, or from about 20 days to about 30 days. In another embodiment, the dosing sessions are spaced apart by about 42 days, about 39 days, about 56 days, about 63 days, about 70 days, about 77 days, about 84 days, or about 91 days, about 98 days. In some embodiments, the dosing sessions are spaced apart by about 84 days. In even another embodiment, the multiple dosing sessions are weekly (about every 7 days), bi-weekly (e.g., about every 14 days), about every 21 days, monthly (e.g., about every 30 days), bi-monthly (e.g., about every 60 days), or every three months. In yet another embodiment, the dosing sessions are monthly dosing sessions (e.g., from about 28 days to about 31 days) and at least three dosing sessions are employed.

In one embodiment, the non-surgical SCS delivery methods, for example, with one of the devices provided herein, are used to treat a patient in need of treatment of macular edema associated with uveitis (e.g., non-infectious uveitis). In one embodiment, SCS administration of a drug (e.g., an anti-inflammatory compound such as a steroid or NSAID) via the methods described herein reduces the vitreous haze experienced by the patient.

In one embodiment, vitreous haze will is assessed via indirect ophthalmoscopy using a standardized photographic scale ranging from 0 to 4, with 0-4 defined below in Table 1 (Nussenblatt 1985 as modified in Lowder 2011, incorporated by reference herein in their entireties). Vitreous haze in another embodiment, is graded from color fundus photographs according to a similar scale.

TABLE 1

| Score | Description |
|---|---|
| 0 | No inflammation |
| +0.5 | Trace inflammation (slight blurring of the optic |
| | disc margins and/or loss of the nerve fiber |
| | layer reflex) |
| +1 | Mild blurring of the retinal vessels and optic nerve |
| +1.5 | Optic nerve head and posterior retina view |
| | obsuration greater than +1 but less than +2 |
| +2 | Moderate blurring of the optic nerve head |
| +3 | Marked blurring of the optic nerve head |
| +4 | Optic nerve head not visible |

The efficacy of the method, in one embodiment, is measured by measuring the patient's mean change from baseline in macula thickness at one or more time points after the patient is treated. For example, at one week, two weeks, three weeks, one month, two months, three months, four months, five months, six months, or more, including all durations in between, after treatment, e.g., with an anti-inflammatory drug delivered non-surgically to the SCS, mean change from baseline in retinal thickness and/or macula thickness is measured.

A decrease in retina thickness and/or macula thickness is one measurement of treatment efficacy of the methods provided herein. For example, in one embodiment, a patient treated by one of the methods provided herein for example with one of the devices described herein experiences a decrease in retinal thickness from baseline (e.g., retinal thickness such as central retinal thickness (CRT) or central subfield thickness (CST) prior to treatment), at any given time point after at least one dosing session (single session or multiple dosing sessions), of at least about 20 μm, or at least about 40 μm, or at least about 50 μm, or at least about 100 μm, or at least about 150 μm or at least about 200 μm, or from about 50-100 μm, or from about 75-200 μm, or from about 100-150 μm, or from about 150-200 μm, and all values in between. In another embodiment, the patient experiences a ≥5%, ≥10%, ≥15%, ≥20%, ≥25%, ≥25%, ≥30%, ≥35%, or ≥40%, decrease in retinal thickness (e.g., CST) subsequent to at least one dosing session.

In one embodiment, the decrease in retinal thickness is measured about 2 weeks, about 1 month, about 2 months, about 3 months, about 6 months, or longer after the at least one dosing session. In another embodiment, the decrease in retinal thickness is measured at least about 2 weeks, at least about 1 month, at least about 2 months, at least about 3 months, at least about 6 months, or longer after the at least one dosing session. In one embodiment, where multiple dosing sessions are employed, a decrease in retinal thickness is sustained by the patient for at least about 2 weeks, at least about 1 month, at least about 2 months, at least about 3 months, at least about 6 months, or longer after each dosing session.

In one embodiment, a macular edema associated with uveitis (e.g., non-infectious uveitis) patient treated by the methods provided herein experiences a decrease in retinal thickness from baseline (i.e., retinal thickness prior to treatment), at any given time point, of from about 20 μm to about 200 μm, at from about 40 μm to about 200 μm, of from about 50 μm to about 200 μm, of from about 100 μm to about 200 μm, or from about 150 μm to about 200 μm. In one embodiment, change in retinal thickness from baseline is measured as a change in CRT or CST, for example, by spectral domain optical coherence tomography (SD-OCT).

In yet another embodiment, the therapeutic response is a change from baseline in macula thickness at one or more time points after the patient is treated. For example, at one week, two weeks, three weeks, one month, two months, three months, four months or more, including all durations in between, after a dosing session, e.g., with an anti-inflammatory drug such as triamcinolone delivered non-surgically to the SCS, change from baseline in macula thickness is measured. A decrease in macula thickness (as compared to prior to treatment) is one measurement of therapeutic response (e.g., by about 10%, or about 20%, or about 30%, or about 40%, or about 50%, or about 60% and more, including all values in between).

Efficacy, in another embodiment, is assessed via a visual acuity measurement at one and/or two months post treatment (e.g., by measuring the mean change in best corrected visual acuity (BCVA) from baseline, i.e., prior to treatment). In one embodiment, a patient treated by one or more of the methods provided herein experiences an improvement in BCVA from baseline, at any given time point (e.g., 2 weeks after administration, 4 weeks after administration, 2 months after at least one dosing session, 3 months after administration), of at least 2 letters, at least 3 letters, at least 5 letters, at least 8 letters, at least 12 letters, at least 13 letters, at least 15 letters, at least 20 letters, and all values in between, as compared to the patient's BVCA prior to the at least one dosing session.

In one embodiment, the patient gains about 5 letters or more, about 10 letters or more, about 15 letters or more, about 20 letters or more, about 25 letters or more in a BCVA measurement after a dosing regimen is complete, for example a monthly dosing regimen, compared to the patient's BCVA measurement prior to undergoing treatment. In even a further embodiment, the patient gains from about 5 to about 30 letters, 10 to about 30 letters, from about 15 letters to about 25 letters or from about 15 letters to about 20 letters in a BCVA measurement upon completion of at least one dosing session, compared to the patient's BCVA measurement prior to the at least one dosing session. In one embodiment, the BCVA gain is about 2 weeks, about 1 month, about 2 months, about 3 months or about 6 months after the at least one dosing session. In another embodiment, the BCVA is measured at least about 2 weeks, at least about 1 month, at least about 2 months, at least about 3 months or at least about 6 months after the at least one dosing session.

In one embodiment, the BCVA is based on the Early Treatment of Diabetic Retinopathy Study (ETDRS) visual acuity charts.

In another embodiment, the patient subjected to a treatment method, e.g., with one of the devices provided herein substantially maintains his or her vision subsequent to the treatment (e.g., a single dosing session or multiple dosing sessions), as measured by losing fewer than 15 letters in a best-corrected visual acuity (BCVA) measurement, compared to the patient's BCVA measurement prior to undergoing treatment. In a further embodiment, the patient loses fewer than 10 letters, fewer than 8 letters, fewer than 6 letters or fewer than 5 letters in a BCVA measurement, compared to the patient's BCVA measurement prior to undergoing treatment.

Decrease in vitreous haze can also be used as a measure of the method's efficacy. Decreases in vitreous haze can be qualitatively and/or quantitatively determined by techniques such as, but not limited to, photographic grading, a scoring system, a multi-point scale, a multi-step scale (e.g. a multi-step logarithmic scale, manual screening by one or more examiners, and/or the like).

In one embodiment, the decrease in vitreous haze is present about 2 weeks, about 1 month, about 2 months, about 3 months or about 6 months after the at least one dosing session. In another embodiment, the decrease in retinal thickness is present at least about 2 weeks, at least about 1 month, at least about 2 months, at least about 3 months or at least about 6 months after the at least one dosing session. In one embodiment, where multiple dosing sessions are employed, a decrease in vitreous haze is experienced by the patient and is present at least about 2 weeks, at least about 1 month, at least about 2 months, at least about 3 months or at least about 6 months after each dosing session.

In a further embodiment, a second drug formulation comprising a VEGF modulator (e.g., a VEGF antagonist) is administered to the eye of the patient via an intravitreal injection. In a further embodiment, the VEGF modulator is ranibizumab, aflibercept or bevacizumab. In some embodiments, the methods provided herein include methods for treating diabetic macular edema (DME) in a patient in need thereof, the method comprising administering an effective amount of a triamcinolone drug formulation to the SCS of the eye of the patient, and further comprising administering a VEGF modulator to the eye of the patient. In further embodiments, the VEGF modulator is administered to the eye via intravitreal injection. In other embodiments, the VEGF modulator is administered to the eye via SCS administration. In some embodiments, the VEGF modulator is aflibercept.

In one embodiment, the methods provided herein provide for effective treatment of a patient who had previously undergone treatment for a posterior ocular disorder, but was unresponsive, or not properly responsive to the prior treatment for the respective posterior ocular disorder. As one of skill in the art will appreciate, a patient unresponsive or not properly responsive to treatment does not exhibit an improvement in a symptom or improvement in a clinical manifestation of macular edema associated with the disorder. In one embodiment, the symptom or clinical manifestation is lesion size, inflammation, edema, visual acuity and/or vitreous haze.

In patients undergoing ocular treatment via shunts or cannulae, or other surgical methods, a marked increase or decrease in intraocular pressure has been reported after the treatment method commences. In one embodiment, the intraocular pressure (IOP) of the patient's eye undergoing treatment for uveitis or macular edema associated with uveitis (e.g., non-infectious uveitis) or macular edema associated with or RVO, 2 minutes, 10 minutes, 15 minutes, 30 minutes or 1 hour after suprachoroidal drug administration according to the devices (e.g., the device 100) and/or the methods disclosed herein, is substantially the same IOP, compared to the IOP of the patient's eye prior to administration of the drug. In one embodiment, the IOP of the patient's eye undergoing treatment for uveitis or macular edema associated with uveitis (e.g., non-infectious uveitis), or macular edema associated with RVO, 2 minutes, 10 minutes, 15 minutes, 30 minutes or 1 hour after suprachoroidal drug administration, varies by no more than 10%, compared to the IOP of the patient's eye prior to administration of the drug. In one embodiment, the IOP of the patient's eye undergoing treatment for the uveitis or macular edema associated with uveitis (e.g., non-infectious uveitis) or macular edema associated with RVO, 2 minutes, 10 minutes, 15 minutes or 30 minutes after suprachoroidal drug administration, varies by no more than 20%, compared to the IOP of the patient's eye prior to administration of the drug. In one embodiment, the IOP of the patient's eye undergoing treatment for uveitis or macular edema associated with uveitis (e.g., non-infectious uveitis), or macular edema associated with RVO, 2 minutes, 10 minutes, 15 minutes or 30 minutes after suprachoroidal drug administration, varies by no more than 10%-30%, compared to the IOP of the patient's eye prior to administration of the drug. In a further embodiment, the effective amount of the drug for treating uveitis or macular edema associated with uveitis (e.g., non-infectious uveitis), or macular edema associated with RVO, comprises an effective amount of an anti-inflammatory drug (e.g., triamcinolone).

In one aspect, the methods described herein relate to the administration of a drug formulation for the treatment of uveitis (infectious or non-infectious), macular edema, macular edema associated with non-infectious uveitis, macular edema associated with infectious uveitis, or macular edema associated with RVO, wherein the majority of the drug formulation is retained in the SCS and/or other posterior ocular tissue, in one or both eyes of a patient in need of treatment of the posterior ocular disorder, for a period of time after the treatment method is completed. Without wishing to be bound by theory, drug formulation retention in the SCS contributes to the sustained release profile of the drug formulations described herein.

The method of treating uveitis (e.g., non-infectious uveitis), macular edema associated with uveitis, macular edema associated with RVO, wet AMD, choroidal neovascularization (CNV), or wet AMD associated with CNV in a human subject in need thereof comprises, in one embodiment, surgically or non-surgically administering a drug formulation to the suprachoroidal space of the affected eye of the human subject, wherein upon administration, the drug formulation flows away from the insertion site and is substantially localized to the posterior segment of the eye, for example to the posterior ocular tissue such as the retina and/or choroid. In one embodiment, the methods provided herein allow for longer retention of the drug in the eye, e.g., the posterior segment of the eye, as compared to intravitreal, topical, parenteral, intracameral or oral administration of the same drug dose.

In one embodiment, the suprachoroidal drug dose sufficient to achieve a therapeutic response in a human subject treated with the non-surgical SCS drug delivery method is less than the intravitreal, parenteral, intracameral, topical, or oral drug dose sufficient to elicit the identical or substantially identical therapeutic response. In a further embodiment, the suprachoroidal drug dose is at least 10 percent less than the oral, parenteral or intravitreal dose sufficient to achieve the identical or substantially identical therapeutic response. In a further embodiment, the suprachoroidal dose is about 10 percent to about 25 percent less, or about 10 percent to about 50 percent less than the oral, parenteral, intracameral, topical, or intravitreal dose sufficient to achieve the identical or substantially identical therapeutic response. Accordingly, in one embodiment, the non-surgical SCS administration method achieves a greater therapeutic efficacy than other routes of administration. In one embodiment, the non-surgical method provided herein comprises inserting a hollow microneedle into the sclera of the eye of the human subject and infusing a drug formulation through the hollow microneedle and into the suprachoroidal space of the eye. As described in more detail below, the drug formulation, in one embodiment, is a solution or suspension of the drug.

In one embodiment, the amount of therapeutic formulation delivered into the suprachoroidal space from the devices described herein is from about 10 μL to about 200 μL, e.g., from about 50 μL to about 150 μL. In another embodiment, from about 10 μL to about 500 μL, e.g., from about 50 μL to about 250 μL, is non-surgically administered to the suprachoroidal space.

The amount of drug delivered within the SCS also may be controlled, in part, by the type of microneedle used and how it is used. In one embodiment, a hollow microneedle is inserted into the ocular tissue and progressively retracted from the ocular tissue after insertion to deliver a fluid drug, where after achieving a certain dosage, the delivery could be stopped by deactivating the fluid driving force, such as pressure (e.g., from a mechanical device such as a syringe) or an electric field, to avoid leakage/uncontrolled deliver of drug. Desirably, the amount of drug being delivered is controlled by driving the fluid drug formulation at a suitable infusion pressure. In one embodiment, the infusion pressure may be at least 150 kPa, at least 250 kPa, or at least 300 kPa. In another embodiment, the infusion pressure is about 150 kPa to about 300 kPa. Suitable infusion pressures may vary with the particular patient or species. In another embodiment, the methods provided herein are carried out with one of the devices described above (e.g, the injector 100) or in PCT/US2014/36590, filed May 2, 2014 and entitled “Apparatus and Method for Ocular Injection,” incorporated by reference herein in its entirety for all purposes.

It should be noted that the desired infusion pressure to deliver a suitable amount of drug formulation might be influenced by the depth of insertion of the microneedle and the composition of the drug formulation. For example, a greater infusion pressure may be required in embodiments wherein the drug formulation for delivery into the eye is in the form of or includes nanoparticles or microparticles encapsulating the active agent or microbubbles. Nanoparticle or microparticle encapsulation techniques are well known in the art

In one embodiment, the non-surgical method of administering a drug to the SCS further includes partially retracting the hollow microneedle after insertion of the microneedle into the eye, and before and/or during the infusion of the drug formulation into the suprachoroidal space. In a particular embodiment, the partial retraction of the microneedle occurs prior to the step of infusing the drug formulation into the ocular tissue. This insertion/retraction step may form a pocket and beneficially permits the drug formulation to flow out of the microneedle unimpeded or less impeded by ocular tissue at the opening at the tip portion of the microneedle. This pocket may be filled with drug formulation, but also serves as a conduit through with drug formulation can flow from the microneedle, through the pocket and into the suprachoroidal space. In some embodiments, the drug formulation includes triamcinolone acetonide and is administered via suprachoroidal administration.

In one embodiment, the methods provided herein allow for greater drug retention in the eye compared to other drug delivery methods, for example, a greater amount of drug is retained in the eye when delivered via the methods provided herein as compared to the same dose delivered via intracameral, sub-tenon, intravitreal, topical, parenteral or oral drug delivery methods. Accordingly, in one embodiment, the intraocular elimination half life (t_{1/2}) of the drug when delivered via the methods described herein is greater than the intraocular t_{1/2} of the drug when the same drug dose is administered intravitreally, intracamerally, topically, parenterally or orally. In a further embodiment, the intraocular t_{1/2} of the drug when administered via the non-surgical SCS drug delivery methods provided herein, is from about 1.1 times to about 10 times longer, or from about 1.25 times to about 10 times longer, or from about 1.5 times to about 10 times longer, or about 2 times to about 5 times longer, than the intraocular t½ of the drug when the identical dosage is administered topically, intracamerally, sub-tenonally, intravitreally, orally or parenterally. In another embodiment, the intraocular Cmax of the drug, when delivered via the methods described herein, is greater than the intraocular Cmax of the drug when the same drug dose is administered intravitreally, intracamerally, sub-tenonally, topically, parenterally or orally. In a further embodiment, the intraocular Cmax of the drug when administered via the non-surgical SCS drug delivery methods provided herein, is at least 1.1 times greater, or at least 1.25 times greater, or at least 1.5 times greater, or at least 2 times greater, or at least 5 times greater, than the intraocular Cmax of the drug when the identical dose is administered topically, intracamerally, intravitreally, orally or parenterally. In one embodiment, the intraocular Cmax of the drug when administered via the non-surgical SCS drug delivery methods provided herein, is about 1 to about 2 times greater, or about 1.25 to about 2 times greater, or about 1 to about 5 times greater, or about 1 to about 10 times greater, or about 2 to about 5 times greater, or about 2 to about 10 times greater, than the intraocular Cmax of the drug when the identical dose is administered topically, intracamerally, sub-tenonally, intravitreally, orally or parenterally. In another embodiment, the mean intraocular area under the curve (AUC0-t) of the drug, when administered to the SCS via the methods described herein, is greater than the intraocular AUC0-t of the drug, when administered intravitreally, intracamerally, sub-tenonally, topically, parenterally or orally. In a further embodiment, the intraocular AUC₀₋ₜ of the drug when administered via the non-surgical SCS drug delivery methods provided herein, is at least 1.1 times greater, or at least 1.25 times greater, or at least 1.5 times greater, or at least 2 times greater, or at least 5 times greater, than the intraocular AUC0-t of the drug when the identical dose is administered topically, intracamerally, sub-tenonally, intravitreally, orally or parenterally. In one embodiment, the intraocular AUC₀₋ₜ of the drug when administered via the non-surgical SCS drug delivery methods provided herein, is about 1 to about 2 times greater, or about 1.25 to about 2 times greater, or about 1 to about 5 times greater, or about 1 to about 10 times greater, or about 2 to about 5 times greater, or about 2 to about 10 times greater, than the intraocular AUC0-t of the drug when the identical dose is administered topically, intracamerally, sub-tenonally, intravitreally, orally or parenterally. In yet another embodiment, the intraocular time to peak concentration (tmax) of the drug, when administered to the SCS via the methods described herein, is greater than the intraocular tmax of the drug, when the same drug dose is administered intravitreally, intracamerally, topically, parenterally or orally.

In one embodiment, the drug formulation comprising the effective amount of the drug (e.g., an anti-inflammatory drug (e.g., a steroid such as triamcinolone or NSAID), once delivered to the SCS, is substantially retained in the SCS over a period of time. For example, in one embodiment, about 80% of the drug formulation is retained in the SCS for about 30 minutes, or about 1 hour, or about 4 hours or about 24 hours or about 48 hours or about 72 hours. In this regard, a depot of drug is formed in the SCS and/or surrounding tissue, to allow for sustained release of the drug over a period of time.

In one embodiment, the suprachoroidal drug delivery methods provided herein result in an increased therapeutic efficacy and/or improved therapeutic response, as compared to oral, parenteral, sub-tenon, and/or intravitreal drug delivery methods of the identical or similar drug dose. In one embodiment, the SCS drug dose sufficient to provide a therapeutic response is about 90%, or about 75%, or about one-half (e.g., about one half or less) the intravitreal, intracameral, topical, oral or parenteral drug dose sufficient to provide the same or substantially the same therapeutic response. In another embodiment, the SCS dose sufficient to provide a therapeutic response is about one-fourth the intravitreal, intracameral, sub-tenon, topical, oral or parenteral drug dose sufficient to provide the same or substantially the same therapeutic response. In yet another embodiment, the SCS dose sufficient to provide a therapeutic response is one-tenth the intravitreal, intracameral, sub-tenon, topical, oral or parenteral drug dose sufficient to provide the same or substantially the same therapeutic response. In one embodiment, the therapeutic response is a decrease in inflammation, as measured by methods known to those of skill in the art. In another embodiment, the therapeutic response is a decrease in number of ocular lesions, or decrease in ocular lesion size. In another embodiment, the therapeutic response is a decrease in fluid accumulation and/or intraocular pressure.

Therapeutic response is measured at a time point post-treatment, for example 5 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks 24 weeks, 30 weeks, 36 weeks, 42 weeks, 48 weeks, or longer post-treatment, and all values in between.

The therapeutic efficacy of the drug formulations delivered by the methods described herein and therapeutic response of the human subject can be assayed by standard means in the art, as known to those of skill in the art. In general, the therapeutic efficacy of any particular drug can be assessed by measuring the response of the human subject after administration of the drug; a drug with a high therapeutic efficacy will show a greater amelioration and/or discontinuation of symptoms than a drug with a lower therapeutic efficacy. In non-limiting examples, the efficacy of the drug formulations provided herein can be measured, for example, by observing changes in pain intensity, changes in ocular lesions (size or number), intraocular pressure, fluid accumulation, inflammation (e.g., by measuring changes in the Hackett/McDonald ocular score), ocular hypertension, and/or visual acuity.

In another embodiment, the efficacy of the therapeutic formulation is measured by observing changes in the measurements according to the Hackett/McDonald ocular scores, inflammation, visual acuity, and/or edema. In another embodiment, the efficacy of the therapeutic formulation is measured, for example, by observing changes in the measurements according to the Hackett/McDonald ocular scores, inflammation, visual acuity, and/or edema.

In one embodiment, the non-surgical administration of an effective amount of a drug formulation to the SCS results to treat uveitis (e.g., non-infectious uveitis), macular edema associated with uveitis, diabetic macular edema (DME), macular edema associated with RVO, wet AMD, CNV, wet AMD associated with RVO, or wet AMD associated with CNV results in a decreased number of deleterious side effects or clinical manifestations in the treated patient as compared to the number of side effects or clinical manifestations caused by the same drug dose administered intravitreally, intracamerally, orally or parenterally. In another embodiment, the non-surgical administration of an effective amount of a drug formulation to the SCS results in a decreased number of one or more deleterious side effects or clinical manifestations, as compared to the deleterious side effects or clinical manifestations caused by the same drug dose administered intravitreally, intracamerally, sub-tenonally, orally or parenterally.

Examples of side effects and clinical manifestations that can be reduced or ameliorated include, but are not limited to, inflammation, gastrointestinal side effects (e.g., diarrhea, nausea, gastroenteritis, vomiting, gastrointestinal, rectal, and duodenal hemorrhage, hemorrhagic pancreatitis, large intestine perforation black or bloody stools, and/or coughing up blood); hematologic side effects (e.g., leucopenia, anemia, pancytopenia and agranulocytosis, thrombocytopenia, neutropenia, pure red cell aplasia (PRCA), deep venous thrombosis easy bruising, and/or unusual bleeding from the nose, mouth, vagina, or rectum); immunologic side effects/clinical manifestations (e.g., immunosuppression, immunosuppression resulting in sepsis, opportunistic infections (herpes simplex virus, herpes zoster, and invasive candidal infections), and/or increased infection); oncologic side effects/clinical manifestations (e.g., lymphoma, lymphoproliferative disease and/or non-melanoma skin carcinoma); renal side effects/clinical manifestations (e.g. dysuria, urgency, urinary tract infections, hematuria, kidney tubular necrosis, and/or BK virus-associated nephropathy); metabolic side effects/clinical manifestations (e.g. edema, hyperphosphatemia, hypokalemia, hyperglycemia, hyperkalemia. swelling, rapid weight gain, and/or enlarged thyroid); respiratory side effects/clinical manifestations (e.g., respiratory infection, dyspnea, increased cough, primary tuberculosis dry cough, wheezing, and/or stuffy nose); dermatologic side effects/clinical manifestations (e.g., acne, rash, dyshidrotic eczema, papulosquamous psoriatic-like skin eruption rash, blisters, oozing, mouth sores, and/or hair loss); muscoskeletal side effects/clinical manifestations (e.g. myopathy and/or muscle pain), hepatic side effects/clinical manifestations (e.g. hepatoxicity and/or jaundice), abdominal pain, increased incidence of first trimester pregnancy loss, missed menstrual periods, severe headache, confusion, change in mental status, vision loss, seizure (convulsions), increased sensitivity to light, dry eye, red eye, itchy eye, and/or high blood pressure. As provided above, the reduction or amelioration of the side effect or clinical manifestation is a reduction or amelioration, as compared to the severity of the side effect or clinical manifestation prior to administration of the drug formulation to the SCS of the eye of the patient, or a reduction or amelioration of the side effect or clinical manifestation in the patient, as compared to the reduction or amelioration experienced upon intravitreal, intracameral, parenteral or oral administration of the same drug.

A wide range of therapeutic formulations, for example those that include one or more drugs and/or cellular therapies may be formulated for delivery to the suprachoroidal space and posterior ocular tissues with the present microneedle devices and methods. As used herein, the term “drug” refers to any prophylactic, therapeutic, or diagnostic agent, i.e., an ingredient useful for medical applications. The drug may be selected from cellular therapeutics, small molecules, biologics such as proteins, peptides and fragments thereof, nucleic acids including vectors encoding nucleic acid gene therapeutics, which can be naturally occurring, synthesized or recombinantly produced. For example, in one embodiment, the drug delivered to the suprachoroidal space with the non-surgical methods described herein is an antibody or a fragment thereof (e.g., a Fab, Fv or Fc fragment). In certain embodiments, the drug is a sub-immunoglobulin antigen-binding molecule, such as Fv immunoglobulin fragment, minibody, diabody, and the like, as described in U.S. Pat. No. 6,773,916, incorporated herein by reference in its entirety for all purposes. In one embodiment, the drug is a humanized antibody or a fragment thereof. In some embodiments, the drug is a drug provided in U.S. Pat. No. 9,636,332 and/or U.S. Patent Publication No. 2018-0042765, the entire contents of each of which are incorporated by reference herein for all purposes.

In one embodiment, the drug delivered to the suprachoroidal space using the non-surgical methods (e.g., microneedle devices and methods) or surgical methods (e.g., via a shunt, stent, or cannula) to treat macular edema associated with uveitis or macular edema associated with RVO or diabetic macular edema (DME) is triamcinolone (e.g., triamcinolone acetonide). In one embodiment, the non-surgical and surgical drug delivery methods disclosed herein are used in conjunction with triamcinolone to treat, prevent and/or ameliorate macular edema associated with uveitis (e.g., non-infectious uveitis or infectious uveitis). In a further embodiment, the macular edema associated with uveitis is macular edema associated with non-infectious uveitis. In some embodiments, the uveitis is anterior, intermediate, posterior, or panuveitis. In some embodiments, the methods provided herein include effective treatments for anterior uveitis, intermediate uveitis, posterior uveitis, and/or panuveitis.

In one embodiment, a VEGF modulator is delivered via one of the devices described herein. In one embodiment, the VEGF modulator is a VEGF antagonist. In one embodiment, the VEGF modulator is a VEGF-receptor kinase antagonist, an anti-VEGF antibody or fragment thereof, an anti-VEGF receptor antibody, an anti-VEGF aptamer, a small molecule VEGF antagonist, a thiazolidinedione, a quinoline or a designed ankyrin repeat protein (DARPin). As described herein, in some embodiments for the treatment of macular edema associated with RVO, an anti-inflammatory drug is delivered to the SCS of the eye of a patient in need thereof, in combination with intravitreal delivery of a VEGF modulator (e.g., VEGF antagonist) to the same eye. In one embodiment, the VEGF antagonist is an antagonist of a VEGF receptor (VEGFR), i.e., a drug that inhibits, reduces, or modulates the signaling and/or activity of a VEGFR. The VEGFR may be a membrane-bound or soluble VEGFR. In a further embodiment, the VEGFR is VEGFR-1, VEGFR-2 or VEGFR-3. In one embodiment, the VEGF antagonist targets the VEGF-C protein. In another embodiment, the VEGF modulator is an antagonist of a tyrosine kinase or a tyrosine kinase receptor. In another embodiment, the VEGF modulator is a modulator of the VEGF-A protein. In yet another embodiment, the VEGF antagonist is a monoclonal antibody. In a further embodiment, the monoclonal antibody is a humanized monoclonal antibody.

In one embodiment, the VEGF modulator is one or more of the following: AL8326, 2C3 antibody, AT001 antibody, HyBEV, bevacizumab (Avastin®), ANG3070, APX003 antibody, APX004 antibody, ponatinib (AP24534), BDM-E, VGX100 antibody (VGX100 CIRCADIAN), VGX200 (c-fos induced growth factor monoclonal antibody), VGX300, COSMIX, DLX903/1008 antibody, ENMD2076, sunitinib malate (Sutent®), INDUS815C, R84 antibody, KD019, NM3, allogenic mesenchymal precursor cells combined with an anti-VEGF antagonist (e.g., anti-VEGF antibody), MGCD265, MG516, VEGF-Receptor kinase inhibitor, MP0260, NT503, anti-DLL4/VEGF bispecific antibody, PAN90806, Palomid 529, BD0801 antibody, XV615, lucitanib (AL3810, E3810), AMG706 (motesanib diphosphate), AAV2-sFLT01, soluble Flt1 receptor, cediranib (Recentin™), AV-951, tivozanib (KRN-951), regorafenib (Stivarga®), volasertib (B16727), CEP11981, KH903, lenvatinib (E7080), lenvatinib mesylate, terameprocol (EM1421), ranibizumab (Lucentis®), pazopanib hydrochloride (Votrient™), PF00337210, PRS050, SPO1 (curcumin), carboxyamidotriazole orotate, hydroxychloroquine, linifanib (ABT869, RG3635), fluocinolone acetonide (Iluvien®), ALG1001, AGN150998, DARPin MP0112, AMG386, ponatinib (AP24534), AVA101, nintedanib (Vargatef®), BMS690514, KH902, golvatinib (E7050), everolimus (Afinitor®), dovitinib lactate (TKI258, CHIR258), ORA101, ORA102, axitinib (Inlyta®, AG013736), plitidepsin (Aplidin®), PTC299, aflibercept (Zaltrap®, Eylea®), pegaptanib sodium (Macugen™, L1900015), verteporfin (Visudyne®), bucillamine (Rimatil, Lamin, Brimani, Lamit, Boomiq), R3 antibody, AT001/r84 antibody, troponin (BLS0597), EG3306, vatalanib (PTK787), Bmab100, GSK2136773, Anti-VEGFR Alterase, Avila, CEP7055, CLT009, ESBA903, HuMax-VEGF antibody, GW654652, HMPLO10, GEM220, HYB676, JNJ17029259, TAK593, XtendVEGF antibody, Nova21012, Nova21013, CP564959, Smart Anti-VEGF antibody, AG028262, AG13958, CVX241, SU14813, PRS055, PG501, PG545, PTI101, TG100948, ICS283, XL647, enzastaurin hydrochloride (LY317615), BC194, quinolines, COT601M06.1, COT604M06.2, MabionVEGF, SIR-Spheres coupled to anti-VEGF or VEGF-R antibody, Apatinib (YN968D1), and AL3818. In addition, delivery of a VEGF antagonist using the microneedle devices and non-surgical methods disclosed herein may be combined with one or more agents listed herein or with other agents known in the art, either in a single or multiple formulations.

In one embodiment, the drug formulation delivered to the SCS of an eye of a patient in need thereof via the methods described herein comprises an effective amount of vascular permeability inhibitor. In one embodiment, the vascular permeability inhibitor is a vascular endothelial growth factor (VEGF) modulator, such as a VEGF antagonist or an angiotensin converting enzyme (ACE) inhibitor. In a further embodiment, the vascular permeability inhibitor is an angiotensin converting enzyme (ACE) inhibitor and the ACE inhibitor is captopril.

In one embodiment, the drug is a drug that inhibits, reduces or modulates the signaling and/or activity of PDGF-receptors (PDGFR). For example, the PDGF antagonist delivered to the suprachoroidal space for the treatment of one or more posterior ocular disorders such as macular edema associated with uveitis (e.g., non-infectious uveitis), macular edema associated with RVO or wet AMD, in one embodiment, is an anti-PDGF aptamer, an anti-PDGF antibody or fragment thereof, an anti-PDGFR antibody or fragment thereof, or a small molecule antagonist. In one embodiment, the PDGF antagonist is an antagonist of the PDGFRU or PDGFRO. In one embodiment, the PDGF antagonist is the anti-PDGF-β aptamer E10030, dasatinib, sunitinib, axitinib, sorefenib, imatinib, imatinib mesylate, nintedanib, pazopanib HCl, ponatinib, MK-2461, pazopanib, crenolanib, PP-121, telatinib, imatinib, KRN 633, CP 673451, TSU-68 (orantinib), Ki8751, amuvatinib, tivozanib, masitinib, motesanib diphosphate, dovitinib, dovitinib dilactic acid, FOVISTA, or linifanib (ABT-869). As described herein, in one embodiment, the PDGF antagonist, for example, one of the PDGF antagonists described above, can be used in the methods for treating macular edema associated with uveitis, macular edema associated with RVO, wet AMD, CNV, wet AMD associated with RVO, or wet AMD associated with CNV via SCS administration. Moreover, in some embodiments, the PDGF antagonist is administered intravitreally in conjunction with SCS administration aflibercept, in a method of treating one of the aforementioned indications.

In a further embodiment, the PDGF antagonist also has VEGF antagonist activity. For example, an anti-VEGF/PDGF-B darpin, dasatinib, dovitinib, Ki8751, telatinib, TSU-68 (orantinib) or motesanib diphosphate are known inhibitors of both VEGF and PDGF, and can be used in the methods described herein. The dual PDGF/VEGF antagonist can also be administered intravitreally in conjunction with non-surgical delivery of aflibercept to the SCS.

In one embodiment, a drug that treats, prevents and/or ameliorates diabetic macular edema is used in conjunction with the devices and methods described herein and is delivered to the suprachoroidal space of the eye. In a further embodiment, the drug is AKB9778, bevasiranib sodium, Cand5, choline fenofibrate, Cortiject, c-raf 2-methoxyethyl phosphorothioate oligonucleotide, DE109, dexamethasone, DNA damage inducible transcript 4 oligonucleotide, FOV2304, iCo007, KH902, MP0112, NCX434, Optina, Ozurdex, PF4523655, SARI 118, sirolimus, SK0503 or TriLipix. In one embodiment, one or more of the diabetic macular edema treating drugs described above is combined with one or more agents listed above or herein or with other agents known in the art.

In one embodiment, the methods and devices provided herein are used to deliver triamcinolone or triamcinolone acetonide to the suprachoroidal space of an eye of a human subject in need of treatment for treating uveitis (e.g., non-infectious uveitis), macular edema associated with uveitis, diabetic macular edema, wet AMD, CNV, wet AMD associated with RVO, or wet AMD associated with CNV. In another embodiment, triamcinolone or triamcinolone acetonide is delivered via one of the methods described herein.

The term “resolution” as used herein refers to a return to a baseline level. For example, in some embodiments, resolution of macular thickening or resolution of macular edema and the like is defined as a macular thickness of less than 300 microns in CST. In some embodiments the resolution of inflammation refers to a score of zero with respect to inflammatory measures in the eye. For example, in some embodiments, resolution of inflammation in the eye refers to a score of 0 of anterior chamber flare and/or anterior chamber cells and/or vitreous haze and/or other measures of eye inflammation known in the art.

The “therapeutic formulation” delivered via the methods and devices provided herein in one embodiment, is an aqueous solution or suspension, and comprises an effective amount of the drug or therapeutic agent, for example, a cellular suspension. In some embodiments, the therapeutic formulation is a fluid drug formulation. The “drug formulation” is a formulation of a drug, which typically includes one or more pharmaceutically acceptable excipient materials known in the art. The term “excipient” refers to any non-active ingredient of the formulation intended to facilitate handling, stability, dispersibility, wettability, release kinetics, and/or injection of the drug. In one embodiment, the excipient may include or consist of water or saline.

The therapeutic formulation delivered to the suprachoroidal space of the eye of a human subject for the treatment of uveitis (e.g., non-infectious uveitis), macular edema associated with uveitis (e.g., non-infectious uveitis), diabetic macular edema, wet AMD, CNV, wet AMD associated with RVO, or wet AMD associated with CNV may be in the form of a liquid drug, a liquid solution that includes a drug or therapy in a suitable solvent, or liquid suspension. The liquid suspension may include microparticles or nanoparticles dispersed in a suitable liquid vehicle for infusion. In various embodiments, the drug is included in a liquid vehicle, in microparticles or nanoparticles, or in both the vehicle and particles. The drug formulation is sufficiently fluid to flow into and within the suprachoroidal space, as well as into the surrounding posterior ocular tissues. In one embodiment, the viscosity of the fluid drug formulation is about 1 cP at 37° C. In some embodiments, the viscosity of the fluid drug formulation is from about 2 to about 40 cPs at 25° C. In further embodiments, the viscosity of the fluid drug formulation is from about 5 to about 20 cPs at 25° C. In further embodiments, the viscosity of the fluid drug formulation is from about 6 to about 15 cPs at 25° C.

In one embodiment, the drug formulation (e.g., fluid drug formulation) includes microparticles or nanoparticles, either of which includes at least one drug. Desirably, the microparticles or nanoparticles provide for the controlled release of drug into the suprachoroidal space and surrounding posterior ocular tissue. In some embodiments, the drug formulation is prepared with starting material comprising microparticles or nanoparticles, either of which includes at least one drug. As used herein, the term “microparticle” encompasses microspheres, microcapsules, microparticles, and beads, having a number average diameter of from about 1 μm to about 200 μm, for example from about 1 to about 100 μm, or from about 1 μm to about 25 μm or from about 1 μm to about 7 μm. “Nanoparticles” are particles having an average diameter of from about 1 nm to about 1000 nm. The microparticles, in one embodiment, have a D₅₀ of about 5 μm or less. In a further embodiment the microparticles have a D₅₀ of less than 5 μm. In a further embodiment, the D₅₀ is about 3 μm or less. In a further embodiment, the D₅₀ is about 2 μm. The microparticles, in one embodiment, have a D₅₀ of about 1 μm to about 5 μm. In another embodiment, the D₅₀ of the particles in the drug formulation is about 2 μm or less. In another embodiment, the D₅₀ of the particles in the drug formulation is about 1000 nm or less. In another embodiment, the D₅₀ of the particles in the drug formulation is about 100 nm to about 1000 nm. In one embodiment, the drug formulation comprises microparticles having a D₉₉ of about 10 μm or less. In one embodiment, the drug formulation comprises microparticles having a D₉₉ of about 1000 nm to about 10 μm. In another embodiment, the D₉₉ of the particles in the formulation is less than about 10 μm, or less than about 9 μm, or less than about 7 μm or less than about 3 μm. In a further embodiment, the microparticles or nanoparticles comprise an anti-inflammatory drug. In a further embodiment, the anti-inflammatory drug is triamcinolone.

Microparticles and nanoparticles may or may not be spherical in shape. “Microcapsules” and “nanocapsules” are defined as microparticles and nanoparticles having an outer shell surrounding a core of another material. The core can be liquid, gel, solid, gas, or a combination thereof. In one case, the microcapsule or nanocapsule may be a “microbubble” or “nanobubble” having an outer shell surrounding a core of gas, wherein the drug is disposed on the surface of the outer shell, in the outer shell itself, or in the core. (Microbubbles and nanobubles may be respond to accoustic vibrations as known in the art for diagnosis or to burst the microbubble to release its payload at/into a select ocular tissue site.) “Microspheres” and “nanospheres” can be solid spheres, can be porous and include a sponge-like or honeycomb structure formed by pores or voids in a matrix material or shell, or can include multiple discrete voids in a matrix material or shell. The microparticles or nanoparticles may further include a matrix material. The shell or matrix material may be a polymer, amino acid, saccharride, or other material known in the art of microencapsulation.

The drug-containing microparticles or nanoparticles may be suspended in an aqueous or non-aqueous liquid vehicle. The liquid vehicle may be a pharmaceutically acceptable aqueous solution, and optionally may further include a surfactant. The microparticles or nanoparticles of drug themselves may include an excipient material, such as a polymer, a polysaccharide, a surfactant, etc., which are known in the art to control the kinetics of drug release from particles.

In one embodiment, the drug formulation further includes an agent effective to degrade collagen or GAG fibers in the sclera, which may enhance penetration/release of the drug into the ocular tissues. This agent may be, for example, an enzyme, such a hyaluronidase, a collagenase, or a combination thereof. In a variation of this method, the enzyme is administered to the ocular tissue in a separate step from-preceding or following-infusion of the drug. The enzyme and drug are administered at the same site.

In another embodiment, the drug formulation is one which undergoes a phase change upon administration. For instance, a liquid drug formulation may be injected through hollow microneedles into the suprachoroidal space, where it then gels and the drug diffuses out from the gel for controlled release.

The therapeutic substance in one embodiment is formulated with one or more polymeric excipients to limit therapeutic substance migration and/or to increase viscosity of the formulation. A polymeric excipient may be selected and formulated to act as a viscous gel-like material in-situ and thereby spread into a region of the suprachoroidal space and uniformly distribute and retain the drug. The polymer excipient in one embodiment is selected and formulated to provide the appropriate viscosity, flow and dissolution properties. For example, carboxymethylcellulose is used in one embodiment to form a gel-like material in the suprachoroidal space. The viscosity of the polymer in one embodiment is enhanced by appropriate chemical modification to the polymer to increase associative properties such as the addition of hydrophobic moieties, the selection of higher molecular weight polymer or by formulation with appropriate surfactants.

The dissolution properties of the therapeutic formulation in one embodiment is adjusted by tailoring of the water solubility, molecular weight, and concentration of the polymeric excipient in the range of appropriate thixotropic properties to allow both delivery through a small gauge needle and localization in the suprachoroidal space. The polymeric excipient may be formulated to increase in viscosity or to cross-link after delivery to further limit migration or dissolution of the material and incorporated drug.

Water soluble polymers that are physiologically compatible are suitable for use as polymeric excipients in the therapeutic formulations described herein, and for delivery via the methods and devices described herein include but are not limited to synthetic polymers such as polyvinylalcohol, polyvinylpyrollidone, polyethylene glycol, polyethylene oxide, polyhydroxyethylmethacrylate, polypropylene glycol and propylene oxide, and biological polymers such as cellulose derivatives, chitin derivatives, alginate, gelatin, starch derivatives, hyaluronic acid, chondroiten sulfate, dermatin sulfate, and other glycosoaminoglycans, and mixtures or copolymers of such polymers. The polymeric excipient is selected in one embodiment to allow dissolution over time, with the rate controlled by the concentration, molecular weight, water solubility, crosslinking, enzyme lability and tissue adhesive properties of the polymer.

In one embodiment, a viscosity modifying agent is present in a therapeutic formulation delivered by one of the methods and/or devices described herein. In a further embodiment, the viscosity modifying agent is polyvinyl alcohol, polyvinyl pyrrolidone, methyl cellulose, hydroxypropyl cellulose, hydroxyethyl cellulose, hydroxymethyl cellulose or hydroxypropyl cellulose. In another embodiment, the formulation comprises a gelling agent such as poly(hydroxymethylmnethacrylate), poly(N-vinylpyrroiidone), polyvinyl alcohol or an acrylic acid polymer such as Carbopol.

In one embodiment, the therapeutic formulation is delivered via one of the methods and or devices described herein as a liposomal formulation.

Liposomes can be produced by a variety of methods. Bangham's procedure (J. Mol. Biol., J Mol Biol. 13(1):238-52, 1965) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation. Each of the patents references in this paragraph is incorporated by reference herein in their entireties for all purposes.

In one embodiment, the liposomal formulation comprises a phospholipid. In a further embodiment, the liposomal formulation comprises a sterol such as cholesterol.

In another embodiment, the liposomal formulation comprises unilamellar vesicles. Unilamellar vesicles can be produced from MLVs by a number of techniques, for example, the extrusion of Cullis et al. (U.S. Pat. No. 5,008,050) and Loughrey et al. (U.S. Pat. No. 5,059,421). Sonication and homogenization can be used to produce smaller unilamellar liposomes from larger liposomes (see, for example, Paphadjopoulos et al., Biochim. Biophys. Acta., 135:624-638, 1967; Deamer, U.S. Pat. No. 4,515,736; and Chapman et al., Liposome Technol., 1984, pp. 1-18). A review of these and other methods for producing liposomes can be found in the text Liposomes, Marc Ostro, ed., Marcel Dekker, Inc., New York, 1983, Chapter 1, the pertinent portions of which are incorporated herein by reference. See also Szoka, Jr. et al., (1980, Ann. Rev. Biophys. Bioeng., 9:467). Each of the references in this paragraph is incorporated by reference herein in their entireties for all purposes.

As described above, the drug formulation delivered to the suprachoroidal space via the methods described herein can be administered with one or more additional drugs. The one or more additional drugs, in one embodiment, are present in the same formulation as the initial drug formulation. In another embodiment, the one or more additional drugs are present in a second formulation. In even a further embodiment, the second drug formulation is delivered to the patient in need thereof via a non-surgical SCS delivery method described herein. Alternatively, the second drug formulation is delivered intravitreally, intracamerally, sub-tenonally, orally, topically or parenterally to the human subject. In one embodiment, a VEGF antagonist is delivered to the suprachoroidal space of the eye of a human subject via one of the methods and/or devices disclosed herein, in conjunction with an anti-inflammatory compound.

As described above, in addition to suprachoroidal delivery, the one or more additional drugs delivered to the human subject can be delivered via intravitreal (IVT) administration (e.g., intravitreal injection, intravitreal implant or eye drops). Methods of IVT administration are well known in the art. Examples of classes of drugs that can be administered via IVT include, but are not limited to: VEGF modulators, PDGF modulators, anti-inflammatory drugs. Accordingly, the methods of the present invention include administrating via IVT one or more of the drugs listed above in combination with one or more drugs disclosed herein administered into the suprachoroidal space using the microneedle device described herein.

### EXAMPLES

The present invention is further illustrated by reference to the following Examples. However, it should be noted that these Examples, like the embodiments described above, are illustrative and are not to be construed as restricting the scope of the invention in any way.

### Example 1. Triamcinolone Formulations for Delivery to the Suprachoroidal Space

Triamcinolone is delivered to the suprachoroidal space using the methods and devices provided herein. The triamcinolone formulation, in one embodiment, is selected from one of the following seven formulations in Table 2. In one embodiment, the formulation is delivered in a volume of 100 μL for total administrated dose of 4 mg TA per administration.

TABLE 2
Triamcinolone (TA) formulations.

| Ingredient | Formulation A | | Formulation B | | Formulation C | | Formulation D | | Formulation E | | Formulation F | | Formulation G | |
|---|---|---|---|---|---|---|---|---|---|---|---|---|---|---|
| Triamcino- | 40 | mg/mL | 40 | mg/mL | 40 | mg/mL | 40 | mg/mL | 40 | mg/mL | 40 | mg/mL | 8 | mg/mL |
| lone | | | | | | | | | | | | | | |
| acetonide | | | | | | | | | | | | | | |
| Particle | D₅₀: ~2 μm | | D₅₀: ~2 μm | | D₅₀: ~2 μm | | D₅₀: ~2 μm | | D₅₀: ~2 μm | | D₅₀: ~2 μm | | D₅₀: ~2 μm | |
| Size | D₉₉: <10 μm | | D₉₉: <10 μm | | D₉₉: <10 μm | | D₉₉: <10 μm | | D₉₉: <10 μm | | D₉₉: <10 μm | | D₉₉: <10 μm | |
| Sodium | 0.64% | w/v | 0.64% | w/v | 0.64% | w/v | 0.55% | w/v | 0.64% | w/v | 0.55% | w/v | 0.55% | w/v |
| Chloride | | | | | | | | | | | | | | |
| Carboxy- | 0.5% | w/v | 0.5% | w/v | 0.5% | w/v | 0.5% | w/v | 0.5% | w/v | 0.5% | w/v | 0.5% | w/v |
| methylcellu- | | | | | | | | | | | | | | |
| lose sodium | | | | | | | | | | | | | | |
| Polysorbate | 0.02% | w/v | 0.015% | w/v | ≥0.015% | w/v | 0.015% | w/v | 0.02% | w/v | 0.02% | w/v | 0.01% | w/v |
| 80 | | | | | | | | | | | | | | |
| KCl | 0.075% | w/v | 0.075% | w/v | 0.075% | w/v | 0.075% | w/v | 0.075% | w/v | 0.075% | w/v | 0.075% | w/v |
| CaCl₂ | 0.048% | w/v | 0.048% | w/v | 0.048% | w/v | 0.048% | w/v | 0.048% | w/v | 0.048% | w/v | 0.048% | w/v |
| (dihydrate) | | | | | | | | | | | | | | |
| MgCl₂ | 0.030% | w/v | 0.030% | w/v | 0.030% | w/v | 0.030% | w/v | 0.030% | w/v | 0.030% | w/v | 0.030% | w/v |
| (hexahy- | | | | | | | | | | | | | | |
| drate) | | | | | | | | | | | | | | |
| Sodium | 0.39% | w/v | 0.39% | w/v | 0.39% | w/v | 0.39% | w/v | 0.39% | w/v | 0.39% | w/v | 0.39% | w/v |
| acetate | | | | | | | | | | | | | | |
| (trihydrate) | | | | | | | | | | | | | | |
| Sodium | 0.17% | w/v | 0.17% | w/v | 0.17% | w/v | 0.17% | w/v | 0.17% | w/v | 0.17% | w/v | 0.17% | w/v |
| citrate | | | | | | | | | | | | | | |
| (dihydrate) | | | | | | | | | | | | | | |

| | | | | | | | |
|---|---|---|---|---|---|---|---|
| NaOH/HCl | Adjust to pH | Adjust to pH | Adjust to pH | Adjust to pH | Adjust to pH | Adjust to pH | Adjust to pH |
| | 6.0-7.5 | 6.0-7.5 | 6.0-7.5 | 6.0-7.5 | 6.0-7.5 | 6.0-7.5 | 6.0-7.5 |

### Example 2. Clinical Evaluation of CLS-TA in Uveitis Patients

A two-arm, randomized, controlled, double-masked, multi-center Phase 3 clinical study was conducted to assess the effectiveness of two doses of CLS-TA in patients with non-infectious uveitis. The non-infectious uveitis included any anatomic subtype (anterior, intermediate, posterior, or panuveitis). The study is referred to as the PEACHTREE study in some embodiments herein. In the study, patients with non-infectious uveitis with macular edema due to their condition and reading <70 ETDRS letters were randomly assigned to an active (SCS administration of CLS-TA) or control (sham SCS injection; no treatment) arm. The subject in the study could have uveitis with any disease diagnosis and etiology in the uveitis spectrum, and with any anatomic location for their uveitis: pan, intermediate, posterior, or anterior. Study medication were administered at day 0 (time 0) and at week 12, and subjects were evaluated every 4 weeks following the baseline treatment. In the study, 160 subjects with macular edema associated with noninfectious uveitis of any anatomic subtype (anterior, intermediate, posterior, or panuveitis) were randomized 3:2 either to suprachoroidal injections of CLS-TA (4.0 mg; n=96) or sham procedures (n=64).

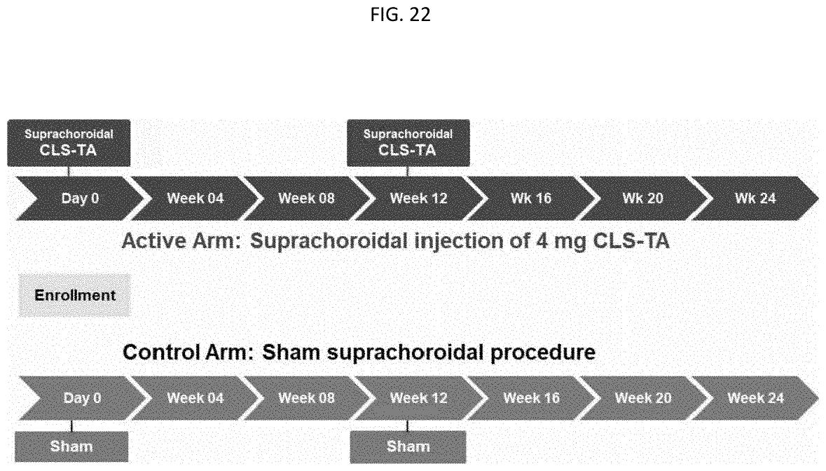

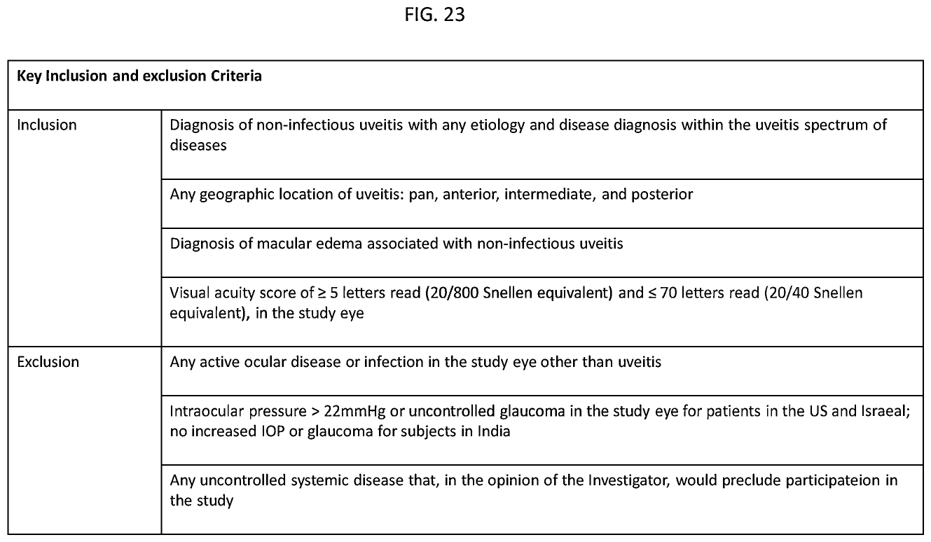

The sham procedure was a sham SCS injection. In the CLS-TA treatment group, subjects were administered 4 mg CLS-TA by SCS injection (100 μL of a drug formulation comprising 40 mg/mL CLS-TA). The 4 mg CLS-TA treatment was administered as a single injection at 2 timepoints, one on day 0 and one on week 12. The sham procedure was also administered at day 0 and at week 12. Subjects were evaluated for a period of at least 6 months starting from day 0. A schematic of the study design is provided in FIG. 22. The key inclusion and exclusion criteria are provided in FIG. 23 and included the following.

Inclusion:
Diagnosis of non-infectious uveitis with any etiology and disease diagnosis within the uveitis spectrum of diseases
Any geographic location of uveitis: pan, anterior, intermediate, and posterior
Diagnosis of macular edema associated with non-infectious uveitis
Visual acuity score of 5 letters read (20/800 Snellen equivalent) and <70 letters read (20/40 Snellen equivalent), in the study eye

Exclusion:
Any active ocular disease or infection in the study eye other than uveitis
Intraocular pressure ≥22 mmHg or uncontrolled glaucoma in the study eye for patients in the US and Israel; no increased IOP or glaucoma for subjects in India
Any uncontrolled systemic disease that, in the opinion of the Investigator, would preclude participation in the study

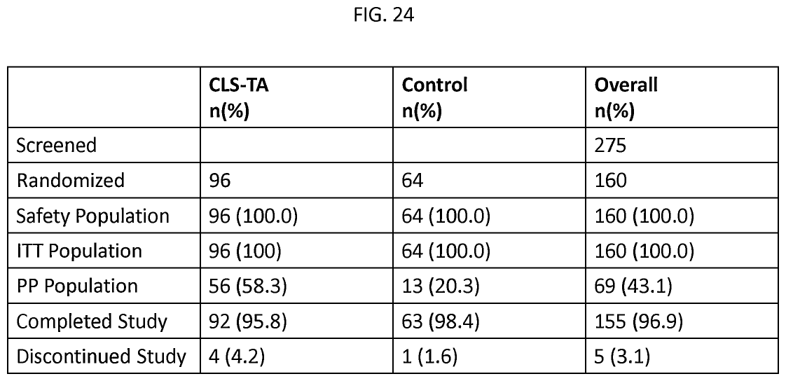

The subject disposition in the CLS-TA and control groups and overall is provided in FIG. 24.

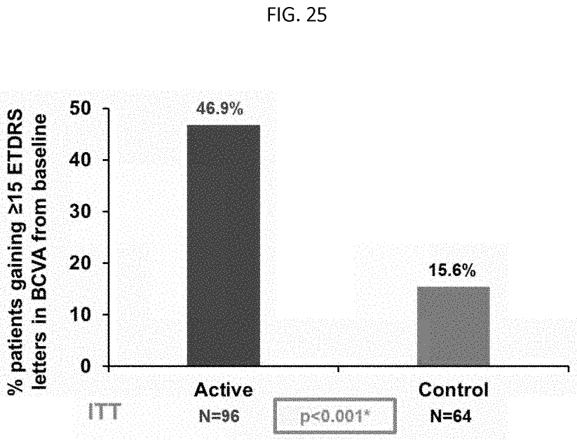

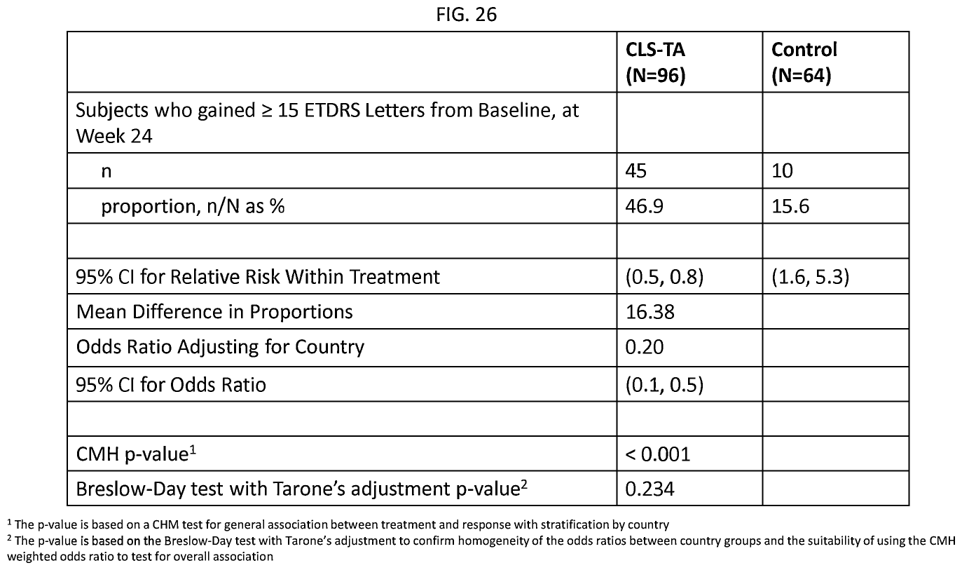

The primary endpoint of the study was the proportion of patients in each arm (CLS-TA treatment group vs. sham procedure control) gaining ≥15 ETDRS letters in BCVA from baseline at week 24. FIG. 25 shows the proportion of patients in each arm that gained at least 15 ETDRS letters in BCVA. Strikingly, 46.9% of subjects in the treatment arm gained at least 15 ETDRS letters in BCVA by week 24. In contrast, only 15.6% of the sham control patients reached at least 15 ETDRS letters in BCVA by week 24. The difference between the two groups was highly statistically significant. The details of the primary endpoint analysis are provided in FIG. 26.

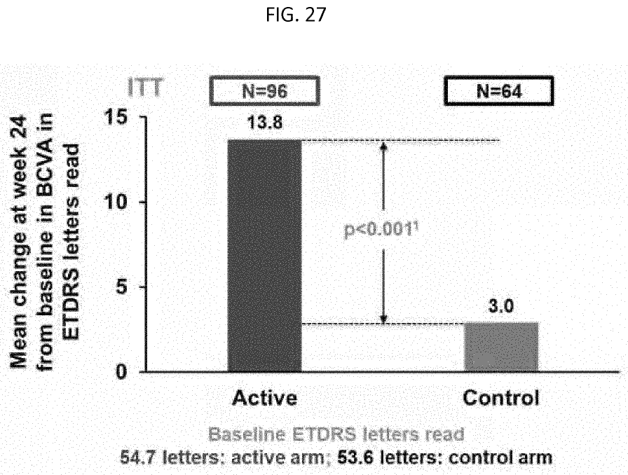

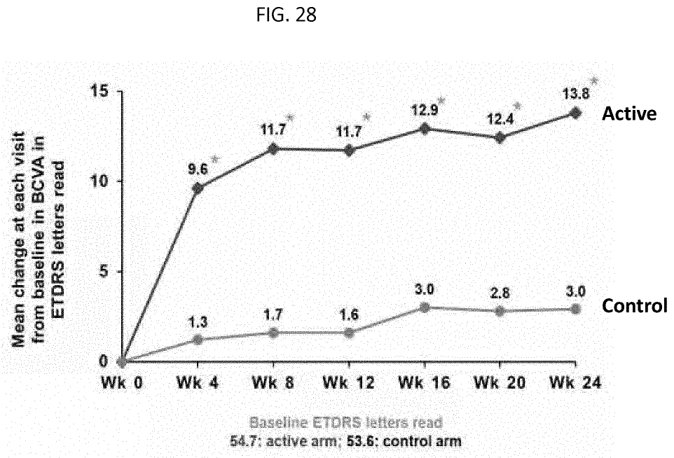

The baseline ETDRS letters read was 54.7. In the CLS-TA arm and 53.6 in the sham control arm. The mean change in BCVA across the entire Active Arm group was an increase of 13.8 letters read, in contrast to just 3.0 letters read in the Control Arm group (p<0.001; FIG. 27). FIG. 28 shows that the mean change in ETDRS letters read consistently increased over months 1, 2, 3, 4, 5, and 6 of the study, and a highly statistically significant difference (p<0.001) compared to the Control Arm at each time point. By week 4, subjects in the CLS-TA group (bars) already had a far larger increase from baseline in ETDRS letters read compared to the control group (lines). The difference between CLS-TA and control treated subjects remained significant at each of weeks 8, 12, 16, 20, and 24.

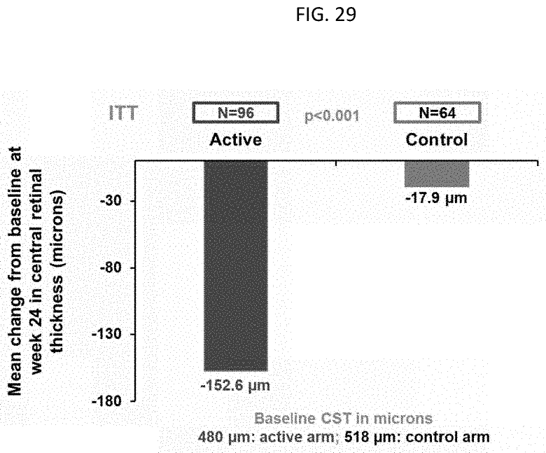

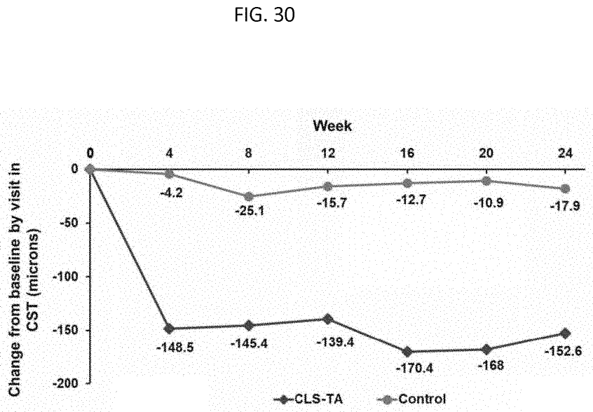

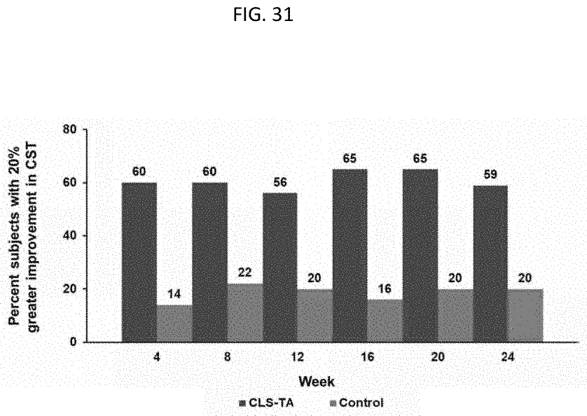

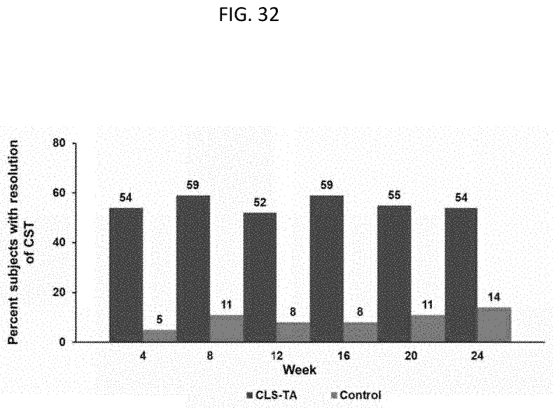

Central retinal thickness was also measured throughout the study. The baseline central retinal thickness (CRT) was 480.0 microns in the active arm, and 518.0 microns in the control arm. FIG. 29 shows the mean change in central retinal thickness in each arm (CLS-TA treatment group vs. sham procedure control) at week 24 of the study. At week 24, the mean change in CST was a decrease in CST of 152.6 microns in the Active Arm, compared to just 17.9 microns in the Control Arm (p<0.001; FIG. 29). Thus, the CLS-TA treatment provided a highly statistically significant difference in the central retinal thickness compared to control treated patients. FIG. 30 shows the mean change in CST from baseline at each visit. Over 50% of the subjects in the Active Arm showed a 20% or greater reduction in excess CST. This was observed at week 4, and sustained through week 24 (FIG. 31). Moreover, approximately 50% or more of subjects in the Active Arm showed resolution of macular thickening (defined as less than 300 microns in CST). This too was observed at week 4 and sustained through week 24 (FIG. 32).

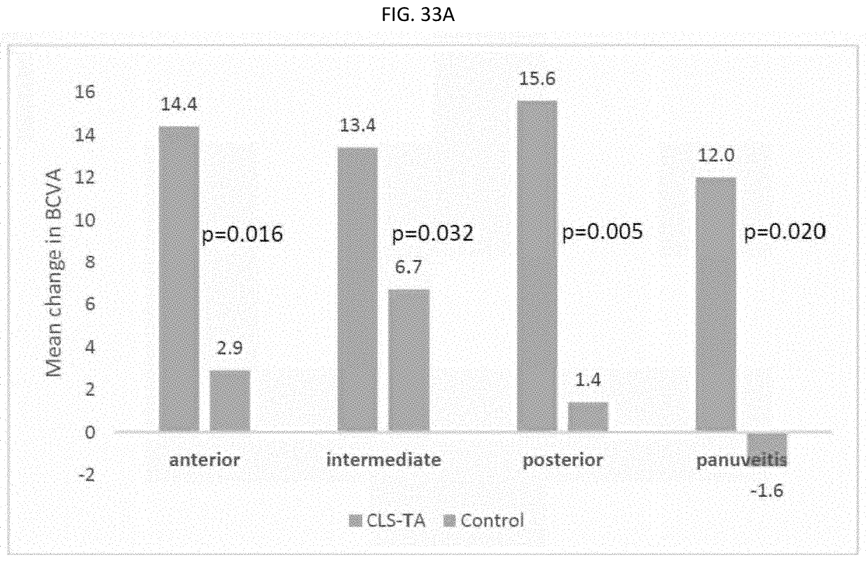

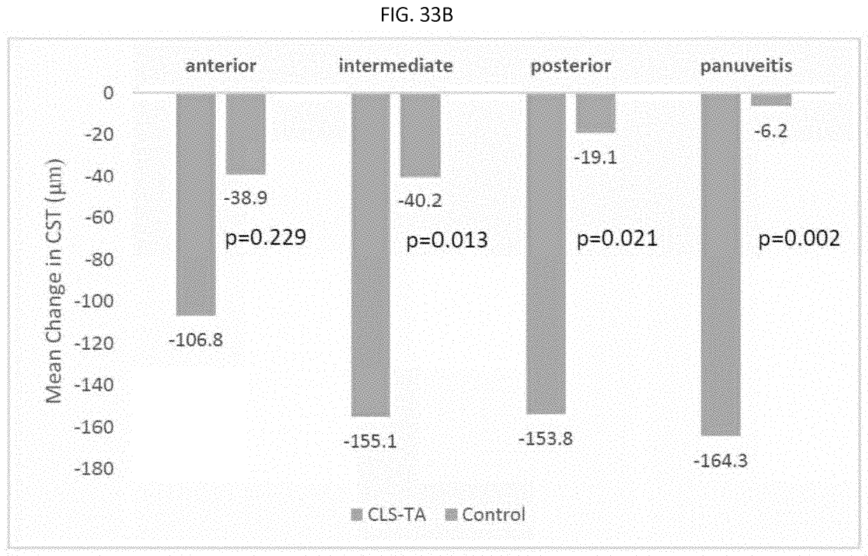

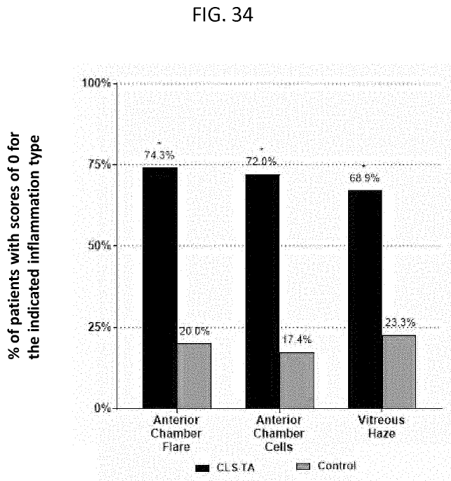

CLS-TA significantly increased BCVA relative to the control arm in all four anatomic subtypes (FIG. 33A). CLS-TA also significantly reduced CST in all anatomic subtypes except for patients with anterior uveitis (FIG. 33B). Accordingly, the Phase 3 study showed that subjects having macular edema associated with noninfectious uveitis, SCS injection of CLS-TA significantly improved patient outcomes irrespective of the anatomic location of uveitis.

The CLS-TA treatment also resulted in a resolution of inflammation. About 70-75% of patients in the Active Arm had scores of zero with respect to anterior chamber flare, cells present in the anterior chamber, and vitreous haze, each of which is a measure of inflammation. In contrast, only about 17-23% of patients in the control arm exhibited a resolution of inflammation (FIG. 34).

Table 3 provides safety summary and safety details. All 160 patients in the study (96:64 active:control) were in the Safety population. There were 3 serious adverse events (SAEs); all three were considered unrelated to treatment. Elevated IOP occurred in 11.5% of subjects in the CLS-TA group and 15.6% of subjects in the control group. All IOP increases in the control group were associated with local corticosteroid rescue treatment. Elevated IOP was managed with topical IOP-lowering medication when treatment was necessary. Cataract AEs were comparable in subjects in the CLS TA arm (7.3%) and control arm (6.3%). In the CLS-TA arm, 13.5% of subjects required rescue medication relative to 71.9% for the control arm. Thus, CLS-TA is a safe and highly effective treatment for non-infectious uveitis.

TABLE 3
Treatment emergent adverse events (TEAEs)

| | CLS-TA | Sham | Total |
|---|---|---|---|
| TEAE | 107 | 64 | 171 |
| TEAE, related | 31 | 8 | 39 |

Together, the results showed that the primary endpoint was met, showing superiority in the proportion of patients in the active (CLS-TA) arm over the control (sham) arm in gaining 15 or more ETDRS letters in BCVA at week 24 from baseline, with P<0.001. Key secondary endpoints of mean change in central retinal thickness from baseline and mean change in BCVA were also met, with P<0.001. Approximately 1 in 2 (˜50%) of the uveitis patients who were administered SCS CLS-TA improved their vision from baseline by 15 or more ETDRS letters. Thus, the results of the Phase 3 clinical study indicated that SCS administration 4 mg of triamcinolone, administered in at least two dosing sessions spaced about 90 days apart, was highly effective, to a highly statistically significant degree compared to control treated patients, in improving vision and CST. In fact, most subjects treated with the claimed dosing regimen experienced a resolution of macular thickening. In addition, the method was highly effective in resolving inflammation of the eye.

In summary, in this pivotal, 6-month, phase 3 trial in subjects with uveitic macular edema of any anatomic location, suprachoroidal injection of CLS-TA met its primary endpoint (% of subjects gaining ≥15 ETDRS letters in BCVA) when compared to sham control while also demonstrating a favorable safety profile.

### Example 3. Extension Study Showing Durability of SCS Administration of CLS-TA

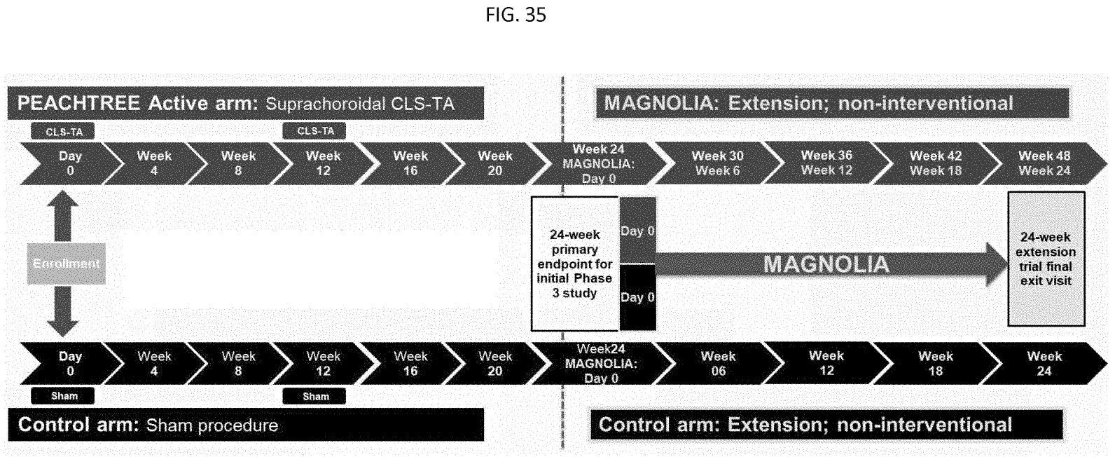

A further clinical study was conducted to assess the durability of the effect of CLS-TA administration to the SCS. This extension study is referred to as the MAGNOLIA study in some embodiments herein. The design of the study was a prospective, non-interventional, masked, observational 24-week extension trial from the 24 week study described above in Example 2. To be eligible, subjects completed the study described in Example 2 and did not receive rescue medication. The exit visit for the initial 24-week study was the first visit for the extension trial. FIG. 35 provides a schematic of the trial design. Only selected clinical sites were used for the extension study, so only about one third of the subjects were enrolled in the extension study. Of the 61 (40%) potential subjects from the initial 24 week study at the sites where the extension study was offered, a total of 33 subjects were enrolled. 28 were in the CLS-TA arm and 5 were in the control arm. Subjects were evaluated every 6 weeks for a maximum of 24 additional weeks; follow up from baseline of the initial 24 week study was therefore 48 weeks. The last dose of CLS-TA (or control) was at week 12 of the initial study. In the extension study, subjects were exited if they qualified to receive rescue medication or at week 48, whichever occurred first.

The objective of the extension trial was to assess the durability of CLS-TA in the uveitis subject population. A Kaplan-Meier evaluation was conducted. 28 CLS-TA patients and 5 Control patients were enrolled, and patient demographics, dispositions, and uveitis disease diagnoses were recorded. All patients were included in the safety population. Uveitis diagnoses included idiopathic, sarcoidosis, HLA-B27-related, juvenile idiopathic arthritis, birdshot retinochoroidopathy, pars plantis, and others. The primary outcome in the extension study was the time to rescue therapy relative to Day 0 of the initial 24 week study.

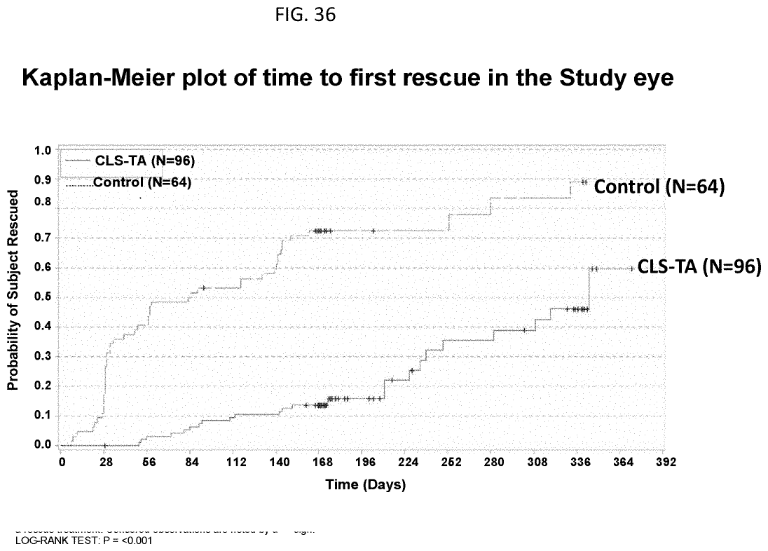

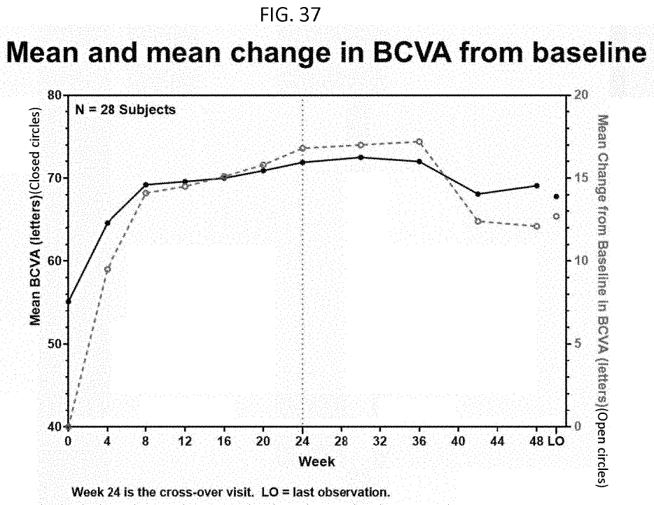

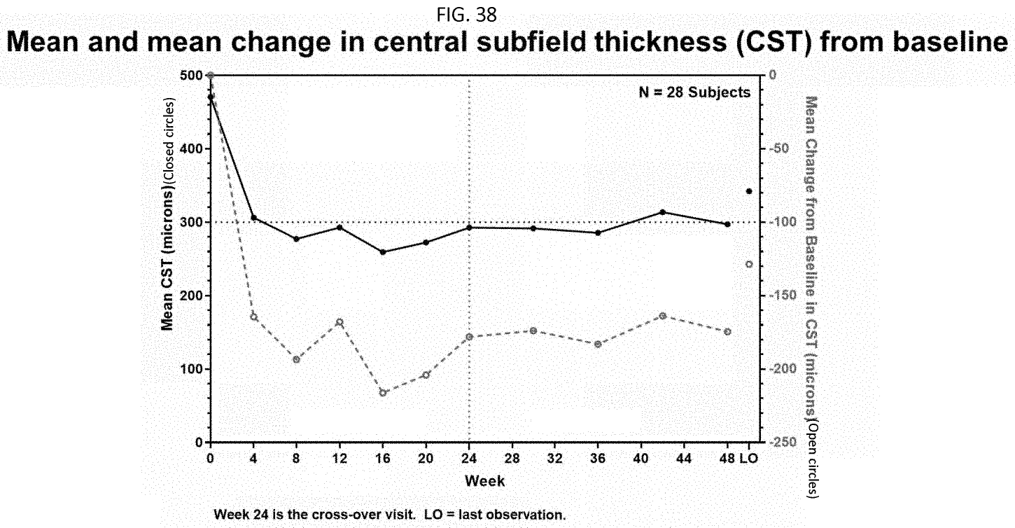

The efficacy results of the study are provided in FIGS. 36-38. FIG. 36 shows the time to first rescue for the treatment of uveitis within 48 weeks, calculated as the number of days between the date of initial randomized treatment with study drug and the visit date of the first reported rescue treatment in the study eye, or date of the final visit of subjects who did not receive a rescue treatment. The data showed that 50% of CLS-TA subjects did not receive any additional medication through week 48. Therefore, half of the enrolled CLS-TA subjects were able to go at least 36 weeks (9 months) from their last (second PEACHTREE protocol-mandated) dose of CLS-TA, indicating strong durability of the treatment. Mean time to rescue therapy was 344 days, from Day 0 of the initial 24 week study. Moreover, the subjects maintained improved vision and improved macular edema throughout. The mean and mean change in BCVA from baseline in the subjects in the initial 24 week study and the 24 week extension study are shown in FIG. 37. Subjects who entered the extension study at week 24 had gained 16-17 ETDRS letters from baseline; those who remained in the extension study through week 48 maintained gains of approximately 12 letters. The mean and mean change in CST from baseline are shown in FIG. 38. Subjects who entered the extension study improved by approximately 170 microns in CST at week 24 from baseline; those who remained in the extension study through week 48 maintained these gains. Thus, the mean improvement in CST through week 48 was approximately 170 microns. Thus, the study showed that the CLS-TA treatment provided a significant, durable improvement in macular edema associated with uveitis.

Safety: There were no serious adverse events related to the study medication; elevations in IOP were consistent with those seen in the initial 24 week study and were low. There were only three elevated IOP adverse events of 10 mmHg or above from baseline in the initial 24 week study, and one in the extension study. No IOP adverse events were above 30 mmHg at any visit during the initial 24 week study, and only one during the extension study. Two elevated IOP adverse reactions were treated with IOP lowering drops, and no surgery was required for any of the IOP increases. There were 3 cataracts in the initial 24 week study and 4 more cataracts in the extension study, for a total of 7 in the active treatment group. There were 25 (7/28) and 20% (1/5) new cataract adverse events in the CLS-TA and control arms, respectively. For the cataract adverse events, there were two cataract surgeries performed.

Taken together, the results of the study showed that SCS administration of CLS-TA provides a safe, effective, and durable treatment for macular edema subjects. Surprisingly, in those patients who did not require rescue treatment, the safety and improvement in vision and macular edema in patients administered CLS-TA lasted for at least 48 weeks from the initial dose of CLS-TA and at least 36 weeks from the last dose of CLS-TA, demonstrating a marked durability of the treatment.

### Example 4. Evaluation of Macular Edema Across Several Studies

A study was conducted to evaluate resolution of macular edema across three clinical trials involving subjects with noninfectious uveitis (NIU) following suprachoroidal injection of 4 mg CLS-TA, an administration approach where drug targets chorioretinal tissues in high amounts, with the potential for demonstrating improvements in anatomical and visual acuity outcomes.

The suprachoroidal space is the region between the choroid and sclera, bound anteriorly by the scleral spur and posteriorly by the optic nerve. Ocular distribution studies conducted in rabbits demonstrate that suprachoroidal injection oftriamcinolone acetonide, TA or CLS-TA, results in high amounts of drug in the posterior tissues of the eye, with the anterior chamber and lens relatively spared. In contrast, currently used local routes of administration expose the anterior segment and lens to substantial steroid concentrations. The suprachoroidal administration approach was used to evaluate the reduction of macular edema in three separate clinical studies of CLS-TA for the treatment of noninfectious uveitis.

Three separate studies evaluated subjects with noninfectious uveitis following a suprachoroidal injection of 4 mg CLS-TA for resolution of macular edema (<300 microns). In DOGWOOD, 17 subjects were administered a single treatment in a randomized, controlled, masked, Phase 2 study with follow-up to 8 weeks. In AZALEA, 38 subjects were administered treatments at Day 0 and Week 12 in an open-label, multi-center, Phase 3 study with follow-up assessments through week 24. In PEACHTREE, 160 subjects were randomized 3:2 to receive either a suprachoroidal injection of CLS-TA (96 subjects) or sham procedure (64 subjects) in a randomized, controlled, double-masked, prospective, multicenter study with follow-up assessments through Week 24. Resolution of macular edema was evaluated at Weeks 4 and 8 in DOGWOOD, and Weeks 4, 8, 12 and 24 in both PEACHTREE and AZALEA. For the present analysis, data were pooled across the three studies.

More than half of the subjects across the three trials that were exposed to 4 mg CLS-TA experienced rapid resolution of edema at Week 4 (54%). The observed effect was sustained through Week 8 with 58% resolution of macular edema. In PEACHTREE and AZALEA, CLS-TA was administered a second time at Week 12 and edema was not present in 52% of subjects. Macular edema resolution was sustained in 56% of subjects at Week 24. Throughout the 24-week PEACHTREE study, corticosteroid-induced elevated IOP was 11.5% in the study eye of subjects in the CLS TA arm. Subjects in the sham procedure (no drug) arm who received local corticosteroid rescue medication subsequently experienced increases of IOP in the study eye at 26.3% (10 of 38 subjects who received local corticosteroid as rescue).

These three clinical trials demonstrate that treatment of noninfectious uveitis with suprachoroidal injections of CLS-TA results in rapid resolution of macular edema as early as Week 4 and is sustained throughout Week 24. The favorable ocular distribution of drug away from the anterior segment following suprachoroidal injection observed in the rabbit model may be reflected in the lower rate of reported elevated IOP in the CLS-TA arm in the PEACHTREE trial. Suprachoroidal administration offers the potential to improve clinically meaningful vision-related outcomes such as macular edema.

### Example 5. Treatment Response Analysis of Visual Acuity and Central Subfield Retinal Thickness Following Suprachoroidal CLS-TA

An analysis was conducted to provide model-based evidence of a relationship between CLS-TA treatment and best corrected visual acuity (BCVA). Data from two Phase 3 trials, PEACHTREE and AZALEA, evaluating suprachoroidal CLS-TA, a triamcinolone acetonide injectable suspension for the treatment of uveitis, were used to develop model-based treatment-response longitudinal models. These models included disease progression effects from both the control arm (sham procedure with no drug) as well as effects following administration of CLS-TA. A covariate analysis was conducted to identify clinically relevant and statistically significant intrinsic and extrinsic factors affecting changes in BCVA response to CLS-TA treatment. The covariates evaluated included race, age, sex, country, baseline BCVA, baseline CST, and anatomic location of ocular inflammation

Data from 198 subjects in PEACHTREE and AZALEA were included. Results of the analysis showed that BCVA exhibits CLS-TA treatment dependent saturable increases over time and CST exhibits CLS-TA treatment dependent saturable declines over time. For the BCVA response model, the baseline BCVA score was significantly influenced by three predictors: baseline CST, age, and study enrollment criteria (AZALEA study had less strict enrollment criteria than the randomized-controlled PEACHTREE trial), while the maximum improvement in BCVA was influenced by the baseline BCVA. Specifically, the maximal increase in BCVA response was significantly influenced by baseline BCVA with greater improvement in subjects with lower baseline BCVA. For the CST response model, the baseline CST measurement was significantly influenced by the study enrollment criteria. The typical subject had a baseline BCVA of 56 ETDRS letters read and a baseline CST of 463 microns. The typical improvement was 12 ETDRS letters read and 114.9 microns decrease in CST following suprachoroidal CLS-TA. In contrast, subjects in the control arm demonstrated an improvement in BCVA of approximately 2 letters and an improvement in CST of approximately 17 microns.

In summary, a model-based treatment-response longitudinal model was developed that characterized changes in both BCVA and CST following administration of CLS-TA. The result of this analysis, based on Phase 3 clinical data, shows that the typical patient will have a 12-letter improvement in BCVA and 114.9 micron decrease in CST after treatment with CLS-TA.

### Example 6. Suprachoroidally Injected CLS-TA Results in Rapid and Sustained Resolution of Macular Edema in a Majority of Uveitis Patients

An analysis was conducted to evaluate the effect of suprachoroidally injected CLS-TA on resolution of macular edema, an administration approach where corticosteroid targets chorioretinal tissues in high amount, thereby providing potentially useful outcomes in a consistent, sustained manner. Ocular distribution of CLS-TA administered by suprachoroidal injection in rabbits reveals steroid largely contained within the posterior segment with the flow of fluid moving dominantly toward the posterior pole, resulting in high amounts of drug in the posterior tissues with relative sparing of the anterior chamber and lens. This unique ocular distribution suggests the potential for improved outcomes with the potential to minimize adverse effects specific to corticosteroid use. Reduction of macular edema was evaluated in three separate studies of suprachoroidally injected CLS-TA in patients with uveitic macular edema.

Three separate studies evaluated resolution of macular edema (<300 microns) following suprachoroidally injected 4 mg CLS-TA in subjects with uveitis in any anatomic location (anterior, intermediate, posterior, or pan). A single treatment was administered in DOGWOOD (randomized, controlled, masked, Phase 2 study of 17 subjects with follow-up to 8 weeks). Treatments were administered at Day 0 and Week 12 with follow up through week 24 in AZALEA (open-label, multicenter, Phase 3 study of 38 subjects) and PEACHTREE (randomized, controlled, double-masked, prospective, multicenter study, of 160 subjects randomized 3:2 to treatment or sham procedures). Data were pooled across the three studies. Macular edema resolution was evaluated at Weeks 4, 8, 12, and 24.

54% of patients experienced a rapid resolution of edema across the 3 studies at week 4. This effect was sustained through week 8 with 58% of cases resolved across the 3 studies. In AZALEA and PEACHTREE, at week 12 when CLS-TA was administered a second time, edema was not present in 52% of patients. Macular edema resolution was sustained in 56% of patients at week 24.

These clinical trials consistently demonstrate that treatment of uveitic macular edema with suprachoroidally injected CLS-TA is associated with rapid improvement in macular edema that is sustained through study completion. The suprachoroidal approach offers the potential to more precisely target chorioretinal tissues and facilitate the treatment of visual loss in patients with uveitic macular edema.

### Example 7. Suprachoroidal CLS-TA Plus Aflibercept Compared to Aflibercept Monotherapy for Diabetic Macular Edema

A study was conducted to evaluate suprachoroidal CLS-TA in combination with intravitreal aflibercept, compared to aflibercept monotherapy for DME. SCS injections target chorioretinal tissues more directly than intravitreal injections while limiting exposure to the anterior chamber, thereby offering potential safety, efficacy, and durability advantages.

The study was a Phase 2, multicenter, randomized, double-masked, controlled, 24-week study in treatment-naïve DME eyes (eligibility: CST ≥300 μm; 20-70 ETDRS letters) randomized 1:1 to CLS-TA+aflibercept or aflibercept monotherapy. Patients in the combination arm (n=36) received CLS-TA and aflibercept at baseline & week 12 (W12). Patients in the monotherapy arm (n=35) received aflibercept at baseline, W4, W8 and W12. Patients in both arms were eligible to receive aflibercept as needed (PRN) at W16 & W20 if predefined re-treatment criteria were met: presence of macular edema (CST ≥340 μm); decrease in BCVA (≥6 letters) from previous visit, or decrease in BCVA (≥10 letters) from best measurement. The study is referred to as the TYBEE study in some embodiments herein.

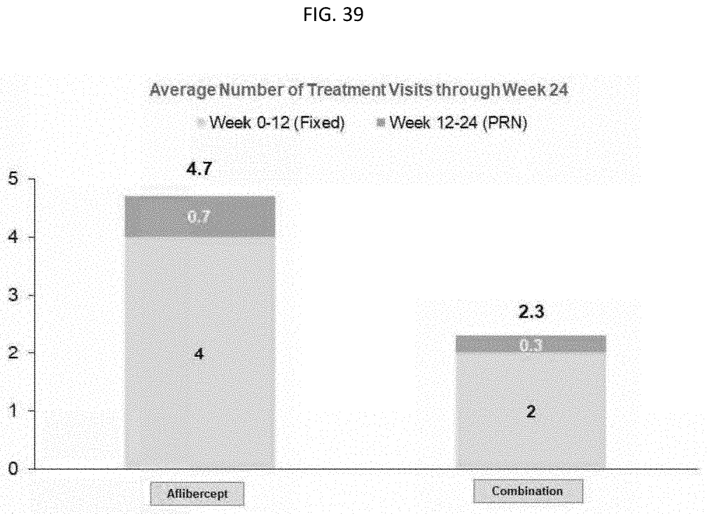

The primary endpoint was mean change in BCVA from baseline at W24. The gain in BCVA letters was +12.3 and +13.5 letters in the combination and monotherapy per protocol arms, respectively. FIG. 39 shows the average number of treatment visits through week 24. Patients in the combination arm received an average of 2.3 treatments: 2 fixed combination treatments at baseline & W12 and a mean 0.3 PRN aflibercept treatments (10 PRN injections/30 patients) at W16 & W20. Patients in the aflibercept monotherapy arm received an average of 4.7 treatments: 4 fixed monthly aflibercept treatments from baseline to W12 and a mean 0.7 PRN aflibercept treatments (23 PRN injections/33 patients) at W16 and W20. Mean CST change was similar between the arms at W24 (combination: −226 μm, aflibercept: −176 μm; p>0.05). Macular edema resolved (<300 μm) in more combination eyes at W4 (combination: 47%, aflibercept: 21%; p<0.01). No treatment-related serious AEs were observed. Elevated IOP was observed in 8.3% (3/36) in the combination arm and (1/35) 2.9% in the monotherapy arm. Cataract was observed in 5.6% (2/36) of the combination arm and 2.9% (1/35) of the aflibercept arm.

Combination therapy with suprachoroidal CLS-TA and aflibercept showed similar visual and anatomic outcomes compared to aflibercept monotherapy for the treatment of DME. Ocular adverse events were low for both arms. Combination dosing was associated with a meaningful reduction in treatment burden.

### Example 8. Clinical Experience with the SCS Microinjector for Suprachoroidal

Injections by Ophthalmologists

This example describes the results of a pooled retrospective analysis on the clinical experience using the SCS Microinjector for suprachoroidal injection of CLS-TA.

A novel microinjector has been developed to deliver a proprietary formulation, CLS-TA, into the suprachoroidal space (SCS) to treat ocular conditions. Successful SCS injections must deliver drug past the sclera into the space between the sclera and choroid without violating the vitreous cavity. Physician experience regarding the SCS Microinjector and proper needle length selection is presented.

A retrospective analysis was performed with data from two uveitis clinical trials evaluating SC injections of CLS-TA administered at Baseline and week 12. All injecting physicians underwent standardized training for proper SC injection techniques: position the needle perpendicular to the globe; 4-4.5 mm posterior to the limbus; compressing the conjunctiva and sclera to first create a dimple, before injecting the drug over several seconds. Unique to the SC injection, injecting physician must actively gauge for a loss in resistance during injection to determine if the SC space has been reached, starting with the 900 μm needle and graduating to the1100 μm needle if needed.

A total of 252 injections (134 patients) were included in the retrospective analysis. 72% (181 of 252) of all injections were completed with the 900 μm needle with the others completed with the 1100 μm needle due to anatomic variations. 83% (98 of 118) of the subjects completed the treatment course (two injections) with the same length needle. SC injections with 900 μm needles were completed at a statistically higher percentage for subjects with intermediate uveitis (81%, 75 of 93), compared to subjects without intermediate uveitis (67%, 106 of 159, p=0.02). There was no statistical significance for various uveitis subtypes (anterior, posterior, pan), uveitis disease course (acute, chronic, or recurrent), uveitis disease duration (limited, <3 months or persistent, ≥3 months), or disease onset (insidious or sudden), relative to needle length.

The preparation and subsequent SC injection of CLS-TA require training for the administering physician specialist and can be accomplished in an in-office setting. Despite the potential limitations of small sample size and the retrospective nature of the analysis, the findings on needle length demonstrate that most suprachoroidal injections in these subjects can be completed with the 900 μm needle.

Publications, patents and patent applications cited herein are specifically incorporated by reference in their entireties. While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

1-54. (canceled)

55. A method of treating macular edema associated with uveitis in a human subject in need thereof, comprising administering to the human subject a composition comprising an effective amount of triamcinolone acetonide to the suprachoroidal space (SCS) of the eye, wherein the method increases a Best Corrected Visual Acuity (BCVA) of at least 15 letters from a baseline measurement prior to the administration of the triamcinolone acetonide to the SCS at 24 weeks.

56. The method of claim 55, wherein the effective amount of triamcinolone acetonide is about 4 mg.

57. The method of claim 55, wherein the triamcinolone acetonide is formulated at 40 mg/mL.

58. The method of claim 57, wherein the composition comprises 0.02% (w/v) polysorbate 80 and 0.5% (w/v) carboxymethylcellulose sodium.

59. The method of claim 58, wherein the triamcinolone acetonide is formulated as a suspension of microparticles having a D₅₀ of 3 μm or less.

60. The method of claim 55, wherein the uveitis is a non-infectious uveitis selected from the group consisting of pan, anterior, intermediate, or posterior uveitis.

61. The method of claim 58, wherein the uveitis is a non-infectious uveitis selected from the group consisting of pan, anterior, intermediate, or posterior uveitis.

62. The method of claim 61, wherein the increase in BCVA from baseline is maintained for 48 weeks.

63. The method of claim 61, wherein administering results in no change in intraocular pressure.

64. The method of claim 61, wherein the administering results in decreased ocular inflammation.

65. The method of claim 61, wherein the administering comprises the use of a microneedle.

66. The method of claim 65, wherein the microneedle is an SCS microinjector.

67. The method of claim 65, wherein the microneedle comprises a 30-gauge needle that is about 900 μm or about 1100 μm in length.

68. The method of claim 65, wherein the administering comprises the steps of: (a) positioning the needle perpendicular to the ocular surface of the eye, between 4-4.5 mm posterior to the limbus of the eye; (b) compressing the conjunctiva and sclera to create a dimple, and (c) injecting the composition over several seconds.

69. The method of claim 67, wherein the administering comprises the steps of: (a) positioning the needle perpendicular to the ocular surface of the eye, between 4-4.5 mm posterior to the limbus of the eye; (b) compressing the conjunctiva and sclera to create a dimple, and (c) injecting the composition over several seconds.

70. The method of claim 58, comprising administering two doses of the composition about 12 weeks apart.

71. A method of treating macular edema associated with uveitis in a human subject in need thereof, comprising administering to the human subject a composition comprising an effective amount of triamcinolone acetonide to the suprachoroidal space (SCS) of the eye, wherein the method reduces a central subfield thickness (CST) of the human subject at 24 weeks from a baseline measurement prior to the administration of the triamcinolone acetonide to the SCS, wherein the reduction in CST is by at least about 100 μm from the baseline.

72. The method of claim 71, wherein the effective amount of triamcinolone acetonide is about 4 mg.

73. The method of claim 71, wherein the triamcinolone acetonide is formulated at 40 mg/mL.

74. The method of claim 73, wherein the composition comprises 0.02% (w/v) polysorbate 80 and 0.5% (w/v) carboxymethylcellulose sodium.

75. The method of claim 74, wherein the triamcinolone acetonide is formulated as a suspension of microparticles having a D₅₀ of 3 μm or less.

76. The method of claim 71, wherein the uveitis is a non-infectious uveitis selected from the group consisting of pan, anterior, intermediate, or posterior uveitis.

77. The method of claim 74, wherein the uveitis is a non-infectious uveitis selected from the group consisting of pan, anterior, intermediate, or posterior uveitis.

78. The method of claim 77, wherein the reduction in CST from baseline is maintained for 48 weeks.

79. The method of claim 77, wherein administering results in no change in intraocular pressure.

80. The method of claim 77, wherein the administering results in decreased ocular inflammation.

81. The method of claim 77, wherein the administering comprises the use of a microneedle.

82. The method of claim 81, wherein the microneedle is an SCS microinjector.

83. The method of claim 81, wherein the microneedle comprises a 30-gauge needle that is about 900 μm or about 1100 μm in length.

84. The method of claim 81, wherein the administering comprises the steps of: (a) positioning the needle perpendicular to the ocular surface of the eye, between 4-4.5 mm posterior to the limbus of the eye; (b) compressing the conjunctiva and sclera to create a dimple, and (c) injecting the composition over several seconds.

85. The method of claim 83, wherein the administering comprises the steps of: (a) positioning the needle perpendicular to the ocular surface of the eye, between 4-4.5 mm posterior to the limbus of the eye; (b) compressing the conjunctiva and sclera to create a dimple, and (c) injecting the composition over several seconds.

86. The method of claim 74, comprising administering two doses of the composition about 12 weeks apart.
